(19) United States
(12) Patent Application Publication
(10) Pub. No.: US 2022/0339135 A1
(43) Pub. Date: Oct. 27, 2022

(54) COMPOSITIONS AND METHODS FOR MODULATING AN INFLAMMATORY RESPONSE

(71) Applicant: Enzychem Lifesciences Corporation, Seoul (KR)

(72) Inventors: Jae Wha Kim, Daejeon (KR); Sun Young Yoon, Daejeon (KR); Ki-Young Sohn, Seoul (KR)

(21) Appl. No.: 17/421,313

(22) PCT Filed: Jan. 7, 2020
(86) PCT No.: PCT/IB2020/000028

§ 371 Date: Jul. 7, 2021

Related U.S. Application Data
(60) Provisional application No. 62/789,485, filed on Jan. 7, 2019.

(51) Int. Cl. A61K 31/231; A61P 37/06

(52) U.S. Cl. CPC A61K 31/231; A61P 37/06

(57) ABSTRACT

In one aspect, methods for modulating an inflammatory response are provided comprising administration to a cell or organism a monoacetyl diacylglycerol compound.

### CROSS-REFERENCES TO RELATED APPLICATIONS

This application is a U.S. national phase application, pursuant to 35 U.S.C. § 371, of PCT/IB2020/000028, filed Jan. 7, 2020, designating the united states, which claims the benefit of U.S. provisional application No. 62/789,485 filed Jan. 7, 2019, the entire contents of which are incorporated herein by reference.

### FIELD

The present invention relates to compositions and methods for modulating inflammatory response, for example, by a cell or in an organism such as a human, comprising administration to the cell or the organism a monoacetyl diacylglycerol compound.

### BACKGROUND

Inflammation is a part of the immune system and occurs in response to injury and infection. For example, inflammation can occur for the defense against invading pathogens, such as bacteria and viruses, and for clearance of damaged tissue. The recruitment of white blood cells, such as leukocytes, serves numerous functions in the inflammatory response. However, this response can also cause tissue damage and contribute to the pathogenesis of numerous diseases.

The innate immune defense mechanism eliminates pathogen-associated molecular pattern (PAMP) molecules and damage-associated molecular pattern (DAMP) molecules. The processes by which this occurs can be generally classified as the following four steps: phagocytosis, necroptosis, netosis, and efferocytosis.

To initiate phagocytosis, a pathogen-associated molecular pattern (PAMP) receptor, a type of a pattern recognition receptor (PRR), located on a membrane surface of a cell recognizes (e.g., binds with) molecules called pathogen-associated molecular pattern (PAMP) molecules, such as a bacterial PAMP molecule, a viral PAMP molecule, a fungal PAMP molecule, or a protozoan PAMP molecule. The PAMP molecules recognized by the PAMP receptor are then internalized by the cell into a phagosome. Once internalized, reactive oxygen species (ROS) are produced by the cell to destroy and eliminate the PAMP molecules.

Also, a damage-associated molecular pattern (DAMP) receptor, another kind of a pattern recognition receptor (PRR), located on a membrane surface of a cell recognizes (e.g., binds with) DAMP molecules. Internalization of DAMP, along with its receptor, can lead to the production and release of chemokines outside the cell, promoting and recruiting additional inflammatory cells. In certain circumstances, the cell may undergo necroptosis, where the cell is programmed to die (e.g., cellular suicide). The death of cells via necroptosis can release intracellular material (e.g., molecules) into the extracellular space. Cells undergoing necroptosis rupture and leak their contents into the intercellular space. Intracellular molecules can then act as DAMP molecules that are recognized by DAMP receptors. This creates a positive feedback loop to amplify necroptosis signaling. This amplified signal causes further release of chemokines to recruit inflammatory cells (e.g., neutrophils) to the site of inflammation.

Neutrophils can form neutrophil extracellular traps (NETs). NETs are structures of decondensed chromatin with histones and intracellular components such as neutrophil elastase (NE), myeloperoxidase (MPO), high mobility group protein B1 (HMGB1), and proteinase 3 (PR3) to remove pathogen-associated molecular pattern (PAMP) molecules and/or damage-associated molecular pattern (DAMP) molecules. The formation of NETs by neutrophils is known as NETosis. Formation of the neutrophil extracellular traps (NETs) can be followed by neutrophil death. Then, dead neutrophils are cleared by phagocytes, e.g., macrophages, through a process known as efferocytosis.

It thus would be desirable to have compositions for modulating inflammatory response, for example, by a cell or in an organism.

### SUMMARY OF THE INVENTION

The present invention is generally directed to compositions and methods of modulating inflammatory response in a cell. The method includes administering a monoacetyl diacylglycerol compound, wherein the administration decreases expression of one or more cytokines, one or more chemokines, or a combination thereof.

In some embodiments, the cell is eukaryotic, for example, a human cell, a mouse cell, a rat cell, a rabbit cell, a dog (canine) cell, a cat (feline) cell, a pig (swine) cell, a cow (bovine) cell, or a non-human primate cell. In some embodiments, the human cell is a macrophage.

In some embodiments, one or more cytokines or chemokines are selected from the group consisting of CXCL8, CXCL2, and IL-6.

In some embodiments, the administration of the composition can decrease the release of one or more damage-associated molecular pattern (DAMP) molecules from a cell.

In another embodiment, the administration increases the trafficking of one or more pattern recognition receptors (PRRs) to a plasma membrane. For example, the one or more PRRs may be suitably selected from the group consisting of a damage-associated molecular pattern receptor, a pathogen-associated molecular pattern receptor, a toll-like receptor, a G protein-coupled receptor, a C-type lectin receptor, or a combination thereof. In certain embodiments, the G protein-coupled receptor may include one or more of rhodopsin-like G Protein-coupled receptors, secretin family receptor proteins, metabotropic glutamate receptors, fungal mating pheromone receptors, cyclic AMP receptors, and frizzled/smoothened G Protein-coupled receptors. In certain embodiments, the G protein-coupled receptor may include a purinergic G protein-coupled receptor. In certain embodiments, the purinergic G protein-coupled receptor is a P2Y1, P2Y2, P2Y4, P2Y6, P2Y11, P2Y12, P2Y13 or P2Y14 receptor.

In one embodiment, modulating the inflammatory response treats disease in a subject in need thereof. In some embodiments, the disease is an inflammatory disease or disorder. In some embodiments, the disease is a disease where there is any abnormal amount of DAMP and/or PAMP. For example, in conditions associated with cell-death, modulation of DAMP would treat or alleviate that condition. Similarly, in conditions associated with infection by a pathogen, modulation of PAMP would treat or alleviate that condition. In some embodiments, the disease or disorder is selected from the group consisting of Chemotherapy-Induced Neutropenia (CIN), Acute Radiation Syndrome (ARS), Psoriasis, Chemoradiation-Induced Oral Mucositis (CRIOM), Acute Lung Injury (ALI), and pneumonia.

In some embodiments, the composition includes a monoacetyl diacylglycerol (MADG). In one embodiment, the MADG binds to a scavenger receptor. Example of scavenger receptors includes, but is not limited to type A, type B, and type C scavenger receptors. By binding to a scavenger receptor, the monoacetyl diacylglycerol modulates scavenger receptor activity.

A monoacetyl diacylglycerol compound, as referred to herein, includes a single acetyl group and a total of two acylglycerol groups. In some embodiments, the monoacetyl diacylglycerol is a compound of Formula I:

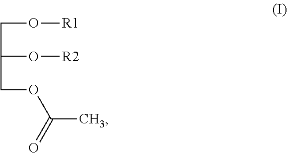

wherein R1 and R2 are independently a fatty acid group comprising 14 to 20 carbon atoms. In one embodiment, the monoacetyl diacylglycerol includes a compound of Formula II:

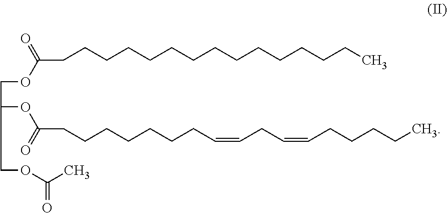 [SMILES: CCCCC/C=C\C/C=C\CCCCCCCC(=O)OC(COC(C)=O)COC(=O)CCCCCCCCCCCCCCC]

The present invention is also directed to a method of modulating an inflammatory response by a cell, wherein the method includes administering to the cell a composition comprising a monoacetyl diacylglycerol.

In some embodiments, the administration of the composition modulates phagocytosis by the cell. For example, modulation of phagocytosis by the cell may accelerate the removal of an apoptotic cell or a necrotic cell from extracellular space. In one embodiment, modulation of phagocytosis by the cell includes an acceleration of removal of a pathogen-associated molecular pattern (PAMP) molecule from extracellular space. In some embodiments, the PAMP molecule is a bacterial PAMP molecule, a viral PAMP molecule, a fungal PAMP molecule, a protozoan PAMP molecule, or a combination thereof.

In other embodiments, the cell may be eukaryotic. For example, the eukaryotic cell is a human cell, a mouse cell, a rat cell, a rabbit cell, a dog (canine) cell, a cat (feline) cell, a pig (swine) cell, a cow (bovine) cell, or a non-human primate cell. In one embodiment, the human cell is a phagocyte. In certain embodiments, the phagocyte is selected from the group consisting of a macrophage, a neutrophil, a monocyte, a mast cell, a dendritic cell, a fibroblast, and an epithelial cell.

In one embodiment, the administration further decreases the expression of one or more cytokines, one or more chemokines, or a combination thereof. The one or more cytokines and the one or more chemokines is selected from the group consisting of CXCL8, CXCL2, and IL-6.

In some embodiments, the administration increases the trafficking of one or more pattern recognition receptors (PRRs) to a plasma membrane. In some embodiments, the one or more PRRs is selected from the group consisting of a damage-associated molecular pattern receptor, a pathogen-associated molecular pattern receptor, a toll-like receptor, a G protein-coupled receptor, a C-type lectin receptor, or a combination thereof. In certain embodiments, the G protein-coupled receptor may include one or more of rhodopsin-like G Protein-coupled receptors, secretin family receptor proteins, metabotropic glutamate receptors, fungal mating pheromone receptors, cyclic AMP receptors, and frizzled/smoothened G Protein-coupled receptors. In other embodiments, the G protein-coupled receptor is a purinergic G protein-coupled receptor. The purinergic G protein-coupled receptor may be a P2Y1, P2Y2, P2Y4, P2Y6, P2Y11, P2Y12, P2Y13 or P2Y14 receptor.

In some embodiments, modulating the inflammatory response treats disease in a subject in need thereof. In some embodiments, the disease is pneumonia.

In some embodiments, the composition (e.g., comprising a monoacetyl diacylglycerol) modulates a scavenger receptor. In one embodiment, the scavenger receptor is a scavenger receptor type A. In one embodiments, the monoacetyl diacylglycerol binds to a scavenger receptor type A. In some embodiments, the monoacetyl diacylglycerol is a compound of Formula I:

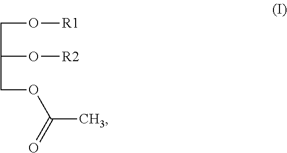

wherein R1 and R2 are independently a fatty acid group comprising 14 to 20 carbon atoms. In some embodiments, the monoacetyl diacylglycerol is a compound of Formula II:

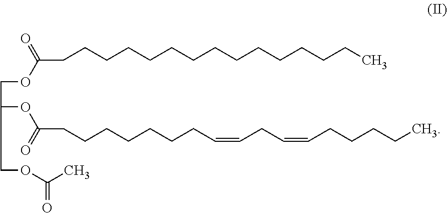 [SMILES: CCCCC/C=C\C/C=C\CCCCCCCC(=O)OC(COC(C)=O)COC(=O)CCCCCCCCCCCCCCC]

The present invention is also directed to a method of modulating an inflammatory response by a cell. The method includes administering to the cell a composition comprising a monoacetyl diacylglycerol, wherein the administration decreases the release of one or more damage-associated molecular pattern (DAMP) molecules from the cell. In one embodiment, an extracellular space includes an increased level of damage-associated molecular pattern (DAMP) molecules. In some embodiments, an extracellular space includes an increased level of pathogen-associated molecular pattern (PAMP) molecules. In some embodiments, the inflammatory response is caused by chemotherapy, radiation, or a combination thereof.

In some embodiments, the method comprises administering to a cell a composition comprising a monoacetyl diacylglycerol, wherein the administration removes one or more pathogen-associated molecular pattern (PAMP), one or more damage-associated molecular pattern (DAMP), or a combination thereof by neutrophil extracellular traps-like structure formed by a neutrophil. In some embodiments, the modulating an inflammatory response by the cell may include modulating NETosis by promoting a formation of NETs-like structure.

In some embodiments, the method comprises administrating to a cell a compound comprising a monoacetyl diacylglycerol, wherein the monoacetyl diacylglycerol binds to scavenger receptor-A (SR-A). In one embodiment, the binding of the monoacetyl diacylglycerol to scavenger receptor-A modulates endocytosis by the cell. In one embodiment, modulation of endocytosis by the cell causes an acceleration of intracellular ROS production. In some embodiments, the administration increases the trafficking of one or more pattern recognition receptors (PRRs) to a plasma membrane. In some embodiments, modulation of endocytosis by the cell results in the acceleration of removal of a pathogen-associated molecular pattern (PAMP) molecule, a damage-associated molecular pattern (DAMP) molecule, or a combination thereof from extracellular space. For example, the process of endocytosis is phagocytosis. In one embodiment, the administration increases the trafficking of one or more pattern recognition receptors (PRRs) to a plasma membrane. In some embodiments, the PAMP molecule is a bacterial PAMP molecule, a viral PAMP molecule, a fungal PAMP molecule, a protozoan PAMP molecule, or a combination thereof.

In some embodiments, the cell is eukaryotic. In one embodiment, the eukaryotic cell is a human cell, a mouse cell, a rat cell, a rabbit cell, a dog (canine) cell, a cat (feline) cell, a pig (swine) cell, a cow (bovine) cell, or a non-human primate cell. In one embodiment, the human cell is a phagocyte. In some embodiments, the phagocyte is selected from the group consisting of a macrophage, a neutrophil, a monocyte, a mast cell, a dendritic cell, a fibroblast, and an epithelial cell.

In one embodiment, the method decreases the expression of one or more cytokines and/or one or more chemokines. In some embodiments, the one or more cytokines and/or one or more chemokines is selected from the group consisting of CXCL8, CXCL2, and/or IL-6.

In one embodiment, the monoacetyl diacylglycerol may comprise a compound of Formula I:

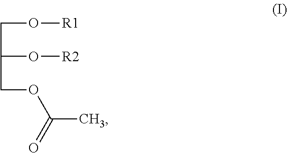

wherein R1 and R2 are independently a fatty acid group comprising 14 to 20 carbon atoms.

In another embodiment, the monoacetyl diacylglycerol may comprise a compound of Formula II:

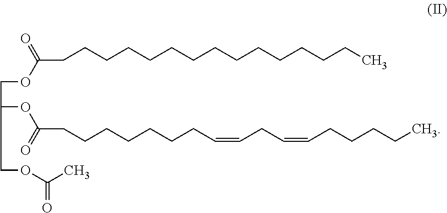 [SMILES: CCCCC/C=C\C/C=C\CCCCCCCC(=O)OC(COC(C)=O)COC(=O)CCCCCCCCCCCCCCC]

In a preferred aspect, methods for treating pneumonia are provided. In one embodiment a method is provided for treating a subject suffering from or susceptible to pneumonia, comprising administering to the subject such as a female or male human a monoacetyl diacylglycerol compound of Formula (I):

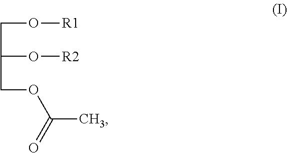

wherein R1 and R2 are independently a fatty acid group comprising 14 to 20 carbon atoms.

Preferably, the subject is administered an effective amount of the compound of Formula II:

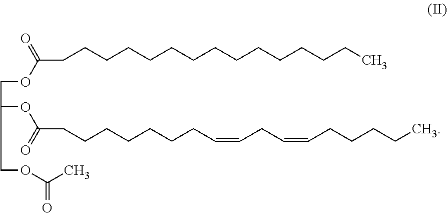 [SMILES: CCCCC/C=C\C/C=C\CCCCCCCC(=O)OC(COC(C)=O)COC(=O)CCCCCCCCCCCCCCC]

Pneumonia may be caused, for example, by a virus, bacteria or fungus. In a particular aspect, pneumonia may be caused by one or more gram-positive or gram-negative bacteria, such as Streptococcus pneumoniae, Pseudomonas aeruginosa, Streptococcus pyogenes, Haemophilus influenza, Staphylococcus aureus, Nocardia sp., Moraxella catarrhalis, Streptococcus pyogenes, Neisseria meningitidis, and/or Klebsiella pneumoniae bacteria. In certain aspects, the pneumonia be caused by bacteria other than Streptococcus pneumoniae, such as one or more gram-positive or gram-negative including Pseudomonas aeruginosa, Streptococcus pyogenes, Haemophilus influenza, Staphylococcus aureus, Nocardia sp. Moraxella catarrhalis, Streptococcus pyogenes, Neisseria meningitidis, and/or Klebsiella pneumoniae bacteria.

In further particular aspects, the subject such as a male or female human may be identified suffering from pneumonia and an effect of a monoacetyl diacylglycerol compound of Formulae (I) or (II) is administered to the identified subject to treat pneumonia. In certain embodiments, a treatment effective amount of a compound of Formulae (I) or (II) is administered to the subject who has been identified as suffering from pneumonia but has not been identified as suffering from a disease or disorder other than pneumonia at the time of such pneumonia treatment.

In a further preferred aspect, methods of attenuating or downregulating a necroptosis signaling by a cell are provided. In one embodiment a method is provided for attenuating or downregulating a necroptosis signaling by a cell, comprising administering the cell a composition including a monoacetyl diacylglycerol compound of Formulae (I) or (II), as described above.

The terms PLAG, EC-18 and 1-palmitoyl-2-linoleoyl-3-acetylglycerol are used interchangeably herein and designate the same compound herein.

Other aspects of the invention are disclosed infra.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1 illustrates confocal microscopy of RAW264.7 cells treated with lipopolysaccharide (LPS) or LPS/1-palmitoyl-2-linoleoyl-3-acetylglycerol (PLAG) using anti-TLR4 (toll-like receptor 4)/MD2 (lymphocyte antigen 96) antibody and Alexa488 conjugated anti-rabbit IgG secondary antibody.

FIG. 2 illustrates confocal microscopy of Raw264.7 cells treated with LPS or LPS/PLAG using Fluorescein isothiocynate (FITC) conjugated-CM-H2DCFDA.

FIGS. 3A-3D illustrates confocal microscopy of Raw264.7 cells treated with LPS and LPS/PLAG. FIG. 3A illustrates confocal microscopy of TLR4/MD2 on the surface of LPS treated and LPS/PLAG treated Raw264.7 cells using anti-TLR4/MD2 antibody and Alexa488 conjugated anti-rabbit igG secondary antibody. FIG. 3B illustrates confocal microscopy of intracellular ROS of LPS treated and LPS/PLAG treated Raw264.7 cells using FITC conjugated-CM-H2DCFDA. FIG. 3C illustrates confocal microscopy of intracellular lysosomes of LPS treated and LPS/PLAG treated Raw264.7 cells using LYSO®-ID Lysosomal Detection Kit. FIG. 3D illustrates confocal microscopy of neutrophil cytosol factor 1 (p47phox) of LPS treated and LPS/PLAG treated Raw264.7 cells using rabbit anti-p47phox.

FIGS. 4A-4H illustrate assessment of LPS-induced acute lung injury (ALI) in control (non-treated), LPS treated, and LPS/PLAG treated mice. FIG. 4A illustrates gross pictures of lungs of control (non-treated), LPS treated, and LPS/PLAG treated mice stained with Evans blue dye. FIG. 4B illustrates histological lung sections of control (non-treated), LPS treated, and LPS/PLAG treated mice stained with hematoxylin and eosin (H&E), with α-neutrophil and α-LPS-specific antibodies. FIG. 4C illustrates lung injury scoring of control (non-treated), LPS treated, and LPS/PLAG treated mice. FIG. 4D illustrates the MPO activity of lungs of control (non-treated), LPS treated, and LPS/PLAG treated mice. FIG. 4E illustrates the number of neutrophils in bronchoalveolar lavage fluid (BALF) after LPS treatment and LPS/PLAG treatment. FIG. 4F illustrates a reverse transcription-polymerase chain reaction (RT-PCR) assessment illustrating the expression of several inflammation-related molecules in BALF and lung tissue after LPS treatment and LPS/PLAG treatment. FIG. 4G illustrates the relative mRNA expression of macrophage inflammatory protein 2 (MIP-2) in BALF after LPS treatment and LPS/PLAG treatment. FIG. 4H illustrates the concentration of secreted MIP-2 in BALF after LPS treatment and LPS/PLAG treatment.

FIGS. 5A-5C illustrate the assessment of Pseudomonas aeruginosa strain K (PAK)-induced bacteria internalization in PAK and PAK/PLAG treated bone marrow-derived macrophages (BMDMs). FIG. 5A illustrates immunofluorescence micrographs of PAK and PAK/PLAG treated BMDMs. FIG. 5B illustrates colony formation assay counting the colony forming unit of intracellular PAK in PAK treated and PAK/PLAG treated BMDMs. FIG. 5C illustrates colony formation assay counting the colony forming unit of intracellular PAK in PAK treated and PAK/PLAG treated A human monocytic cell line (THP-1)cells.

FIGS. 6A and 6B illustrate assessment of clearance of PAK in adriamycin hydrochloride (doxorubicin) and cyclophosphamide (AC regimen)-treated and AC regimen/PLAG treated BALB/c mice model. FIG. 6A illustrates an experimental scheme for the evaluation of PLAG's therapeutic efficacy on AC regimen-treated immunocompromised BALB/c mice model with PAK infection. FIG. 6B illustrates the number of colonies per unit in BALF in AC regimen and AC regimen/PLAG treated BALB/c mice.

FIGS. 7A and 7B illustrate the assessment of intracellular trafficking of G protein-coupled receptor (GPCR) in imiquimod (IMQ) treated and IMQ/PLAG treated HaCaT cells. FIG. 7A illustrates confocal microscopy of adenosine A2A receptor (ADORA2A) on the surface of IMQ and IMQ/PLAG treated HaCaT cells. FIG. 7B illustrates confocal microscopy of intracellular ROS of IMQ treated and IMQ/PLAG treated HaCaT cells.

FIGS. 8A and 8B illustrate the assessment of GPCR related mitogen-activated protein kinase (MAPK) activity in IMQ and IMQ/PLAG treated differentiated HaCaT cells. FIG. 8A illustrates western blot analysis illustrating phosphorylation of ERK, JNK and P38MAPK after 0, 20 and 60 minutes of IMQ treatment. FIG. 8B illustrates western blot analysis illustrating attenuation of IMQ-treated phosphorylation of the extracellular signal-regulated kinase (ERK), c-Jun N-terminal kinases (JNK), p38MAPK by PLAG.

FIG. 9 illustrates concentration of MIP-2, interleukin 6 (IL-6) and chemokine (C-X-C motif) ligand 8 (CXCL8) after IMQ treatment and IMQ/PLAG treatment (upper row, A, B, and C, respectively) and the dependency of CXCL8 expression on MAPK signaling pathway (lower row, D, E, and F, respectively) when cells were treated by MAPK inhibitors.

FIGS. 10A-10H illustrate the assessment of IMQ-induced psoriasis in IMQ and IMQ/PLAG treated BALB/c mice. FIG. 10A illustrates an experimental scheme for evaluation of PLAG's therapeutic efficacy on IMQ-induced psoriasis-like skin inflammation. FIG. 10B illustrates photographs of back skin tissues of control (non-treated), IMQ treated, and IMQ/PLAG co-treated BALB/c mice. FIG. 10C illustrates the scoring of control (non-treated), IMQ treated, and IMQ/PLAG co-treated BALB/c mice. FIG. 10D illustrates back skin thickness of control (non-treated), IMQ treated, and IMQ/PLAG co-treated BALB/c mice. FIG. 10E illustrates ear skin thickness of control (non-treated), IMQ treated, and IMQ/PLAG co-treated BALB/c mice. FIG. 10F illustrates the back skin of control (non-treated), IMQ treated, and IMQ/PLAG co-treated BALB/c mice stained with H&E. FIG. 10G illustrates back skin of control (non-treated), IMQ treated, and IMQ/PLAG co-treated BALB/c mice stained with neutrophil antibodies. FIG. 10H illustrates back skin of control (non-treated), IMQ treated, and IMQ/PLAG co-treated BALB/c mice stained with interleukin 17 (IL-17) antibodies.

FIGS. 11A and 11B illustrate the assessment of monosodium urate (MSU)-induced DAMP molecules and LDH release in the supernatant of MSU-treated and MSU/PLAG treated THP-1 cells. FIG. 11A illustrates western blot analysis of high mobility group box 1 (HMGB1), S100 calcium-binding protein A8 (S100A8), and S100 calcium-binding protein A9 (S100A9) in the supernatant of THP-1 cells after MSU treatment and MSU/PLAG treatment. FIG. 11B illustrates relative cytosolic enzyme lactate dehydrogenase (LDH) release in the supernatant after MSU treatment and MSU/PLAG treatment.

FIGS. 12A and 12B illustrate the assessment of MSU-induced purinoceptor 6 (P2Y6) receptor trafficking in MSU-treated and MSU/PLAG treated THP-1 cells. FIG. 12A illustrates confocal microscopy of P2Y6 receptors after MSU treatment and MSU/PLAG treatment. FIG. 12B illustrates confocal microscopy of lysosomal activity after MSU treatment and MSU/PLAG treatment.

FIGS. 13A and 13B illustrate phosphorylation of receptor-interacting serine/threonine-protein kinase 1 (RIPK1), receptor-interacting serine/threonine-protein kinase 3 (RIPK3) and mixed lineage kinase domain-like (MLKL) in MSU-treated and MSU/PLAG treated THP-1 cells. FIG. 13A illustrates western blot analysis illustrating the phosphorylation of RIPK3 (p-RIPK3) and MLKL (p-MLKL) after MSU treatment and MSU/PLAG treatment. FIG. 13B illustrates western blot analysis illustrating the phosphorylation of receptor-interacting serine/threonine-protein kinase 1 (p-RIPK1) and receptor-interacting serine/threonine-protein kinase 3 (p-RIPK3) by PLAG in a dose-dependent manner.

FIGS. 14A-14C illustrate the assessment of NETosis of control (non-treated), PAK treated, and PAK/PLAG treated bone marrow-derived cells. FIG. 14A illustrates an experimental scheme for the evaluation of PLAG's efficacy on the NETosis of PAK-treated BMDM. FIG. 14B illustrates the NET formation of neutrophil after PAK treatment and PAK/PLAG treatment of BMDM. FIG. 14C illustrates formation of extracellular DNA-elastase complex after PAK treatment and PAK/PLAG treatment of BMDM.

FIGS. 15A-15C illustrate the assessment of NETosis of control (non-treated), PAK treated, and PAK/PLAG treated BALF derived cells. FIG. 15A illustrates an experimental scheme for the NETosis of BALF derived cells in PAK introduced mice. FIG. 15B illustrates the NET formation of neutrophil after PAK treatment and PAK/PLAG treatment of BALF derived cells. FIG. 15C illustrates formation of extracellular DNA-elastase complex after PAK treatment and PAK/PLAG treatment of BALF derived cells.

FIGS. 16A-16D illustrates assessment of intracellular calcium mobilization in control (non-treated), dimethyl sulfoxide (DMSO) treated, PLAG treated and ionomycin treated differentiated human leukemia line (dHL-60) cells. FIG. 16A illustrates the relative level of cytosolic calcium of dHL-60 cells over time after PLAG treatment. FIG. 16B illustrates the relative level of cytosolic calcium of differentiated human leukemia (dHL-60) cells over time after ionomycin treatment. FIG. 16C illustrates western blot analysis of citrullinated histone H3 in dHL-60 cells over time after ionomycin treatment and PLAG treatment. FIG. 16D illustrates the relative level of cytosolic calcium of dHL-60 cells over time after U73122 (phospholipase c inhibitor) treatment of PLAG treated dHL-60 cells in a dose-dependent manner.

FIGS. 17A-17B illustrate the assessment of intracellular calcium mobilization in IMQ treated and IMQ/PLAG treated dHL-60 cells under extracellular calcium-free condition and extracellular calcium-containing condition. FIG. 17A illustrates relative intracellular calcium levels in dHL-60 cells under extracellular calcium-free conditions after IMQ treatment and IMQ/PLAG co-treatment. FIG. 17B illustrates relative intracellular calcium levels in dHL-60 cells under extracellular calcium containing condition after IMQ treatment and IMQ/PLAG co-treatment.

FIG. 18 illustrates confocal microscopy of extracellular DNA-elastase complex formed by NETosis after IMQ treatment and IMQ/PLAG treatment.

FIGS. 19A-19C illustrate clearance of apoptotic neutrophils in control (non-treated), 50 μg/ml of PLAG treated and 10 μg/ml of PLAG treated differentiated HL60 and THP-1 cells. FIG. 19A illustrates efferocytic index over time after PLAG treatment in a dose-dependent manner. FIG. 19B illustrates the clearance of apoptotic neutrophils over time after PLAG treatment in a dose-dependent manner. FIG. 19C illustrates confocal microscopy of apoptotic cells with or without PLAG treatment.

FIGS. 20A and 20B illustrate schematics of PLAG delivery from the intestinal lumen to lymphatic vessels and assembly of chylomicrons. FIG. 20A illustrates a schematic of PLAG delivery from the intestinal lumen to lymphatic vessels. FIG. 20B illustrates a schematic of the assembly of chylomicrons and their delivery to lymphatic vessels.

FIGS. 21A-21E illustrate assessment of PLAG uptake in cisterna chyli. FIG. 21A illustrates PLAG detected in cisterna chyli at a time course. FIG. 21B illustrates absorbance measured from PLAG in cisterna chyli at a time course. FIG. 21C illustrates PLAG detected in cisterna chyli within 1 hour in a dose-dependent manner. FIG. 21D illustrates absorbance measured from PLAG in cisterna chyli in a dose-dependent manner. FIG. 21E illustrates 28.2 mg PLAG found in the lymph fluid as a result of after administration of 62.5 mg PLAG.

FIG. 22 illustrates the change of ¹⁴C radioactivity concentration from PLAG in blood and lymph fluid over time after administration thereof.

FIG. 23 illustrates tissue distribution of PLAG after single oral administration of PLAG using whole-body autoradiography of ¹⁴C.

FIG. 24 illustrates multiple routes and the cumulative amount of PLAG excretion measured by cumulative radioactivity.

FIG. 25 illustrates the average particle size and size distribution of phosphatidylcholine (POPC) and PLAG determined by dynamic light scattering (DLS) and transmission electron microscopy (TEM).

FIGS. 26A-26F illustrates the biological activity of PLAG depending on lipoprotein lipase (LPL) and glycosylphosphatidylinositol-anchored high-density lipoprotein binding protein 1 (GPI-HBP1). FIG. 26A illustrates the interaction between LPL, GPI-HBP1, and chylomicron in normal cells, LPL silenced cells, and GPI-HBP1 silenced cells. FIG. 26B illustrates the phagocytosis of PAK aided by the capture of chylomicron by GPI-HBP1 and LPL. FIG. 26C illustrates the RT-PCR assessment of cells wherein genes LDL and GPI-HBP1 are silenced. FIG. 26D illustrates the phagocytosis rate of control cells, LPL silenced cells, GPI-HBP1 silenced cells after PAK treatment and PAK/PLAG co-treatment. FIG. 26E illustrates confocal microscopy of control cells, LPL silenced cells, GPI-HBP1 silenced cells after PAK/PLAG co-treatment during phagocytosis. FIG. 26F illustrates the down-regulation of chemokine MIP-2 and cytokine IFN-β in the LPS treated macrophage cells by PLAG.

FIGS. 27A-27C illustrates the criticality of acetylated glycerol during monoacetyl diacylglycerol mediated phagocytosis. FIG. 27A illustrates the number of colony forming units of the intracellular PAK of PAK treated cells, PAK/PLAG treated cells, and PAK/palmitic linoleic hydroxyl glycerol (PLH)-treated cells. FIG. 27B illustrates confocal microscopy of PAK treated cells, PAK/PLAG treated cells and PAK/PLH treated cells. FIG. 27C illustrates the number of colony forming units of PAK in BALF of PAK treated cells, PAK/PLAG treated cells and PAK/PLH treated cells.

FIGS. 28A-28C illustrate optimal biological activities of PLAG. FIG. 28A illustrates six exemplary glycerols with its chemical name, chemical structure, molecular formula, and molecular weight. FIG. 28B illustrates the number of colony forming units of the intracellular PAK of cells treated by six different glycerols. FIG. 28C illustrates confocal microscopy of PAK treated cells, PAK/1-lauryl-2-linoleoyl-3-acetyl-glycerol (LLAG) treated cells, PAK/1-myristyl-2-linoleoyl-3-acetyl-glycerol (MLAG) treated cells, PAK/PLAG treated cells, PAK/1-stearyl-2-linoleoyl-3-acetyl-glycerol (SLAG) treated cells and PAK/1-arachidyl-2-linoleoyl-3-acetyl-glycerol (ALAG) treated cells.

FIGS. 29A-29C illustrate a comparison of PLAG with other monoacetyl diacylglycerols in LPS induced acute lung injury (ALI). FIG. 29A illustrates schematic structures of PLAG, PLH, hydroxyl linoleic hydroxyl glycerol (HLH), linoleic acid (LA) and palmitoleic acid (PA). FIG. 29B illustrates neutrophil counts in BALF after LPS treatment, LPS/PLAG treatment, LPS/PLAG treatment, LPS/PLH treatment, LPS/HLH treatment, LPS/LA treatment, and LPS/PA treatment. FIG. 29C illustrates confocal microscopy of LPS induced, LPS/PLAG treated and PLH/LPS treated cell surfaces spanning TLR4 using anti-TLR4/MD2 antibodies.

FIGS. 30A and 30B illustrate uptake of triglyceride (TG) at peripheral tissues in a streptozotocin (STZ)-induced mice model. FIG. 30A illustrates plasma LPL activity of control, STZ treated cell, STZ/PLAG treated cells. FIG. 30B illustrates the expression of apolipoprotein B (ApoB) protein100 and ApoB protein48 in portal vein after STZ treatment and STZ/PLAG co-treatment.

FIGS. 31A-31C illustrates the dose-dependent alleviation of accumulated triglyceride in the liver by PLAG. FIG. 31A illustrates an experimental scheme for the evaluation of PLAG's therapeutic efficacy on STZ-treated liver steatosis. FIG. 31B illustrates livers of control, STZ-treated, STZ/PLAG co-treated, STZ/PLAG post-treated mice. FIG. 31C illustrates H&E stained liver tissues of control, STZ treated, STZ/PLAG 50 mpk treated, and STZ/PLAG 250 mpk treated mice.

FIGS. 32A-32C illustrate assessment of LPL expression in muscle cells in control (non-treated), STZ treated, and STZ/PLAG treated mice. FIG. 32A illustrates muscle LPL mRNA expression of control, STZ-treated, and STZ/PLAG treated mice. FIG. 32B illustrates immunohistochemistry stained LPL in the muscle of control, STZ and STZ/PLAG treated mice. FIG. 32C illustrates muscle TG content of control, STZ-treated, and STZ/PLAG treated mice.

FIGS. 33A-33C illustrate assessment of hepatic steatosis in control, STZ treated, STZ/PLAG treated, and STZ/PLH treated mice. FIG. 33A illustrates livers of control, STZ-treated, STZ/PLAG treated, and STZ/PLH treated mice. FIG. 33B illustrates the change in body weight for control, STZ-treated, STZ/PLAG treated, and STZ/PLH treated mice. FIG. 33C illustrates H&E stained liver tissues of control, STZ treated, STZ/PLAG treated, and STZ/PLH treated mice.

FIGS. 34A-34C illustrate the cluster of differentiation 36 (CD36)-independent of PLAG in reducing MSU crystal-induced CXCL8. FIG. 34A illustrates TG hydrolysis and free fatty acid (FFA) uptake by CD36. FIG. 34B illustrates western blot analysis of cells wherein a gene CD36 silenced. FIG. 34C illustrates PLAG's efficacy towards the CXCL8 decrease in both control and CD36 silenced cells.

FIG. 35 illustrates flow cytometric analysis illustrating PLAG's efficacy towards the acceleration of P2Y6 receptor endocytosis in both control and CD36 silenced cells.

FIGS. 36A and 36B illustrate clearance of DAMP molecules induced by radiation in control (non-treated), radiation treated, radiation/PLAG 50mpk treated, and radiation/PLAG 250mpk treated mice. FIG. 36A illustrates western blot analysis of HMGB1 and S100A9 in the supernatant after radiation, MSU/PLAG 50mpk treatment, and MSU/PLAG 250mpk treatment. FIG. 36B illustrates relative gene expressions of HMGB1 and S100A9 in the supernatant after radiation, MSU/PLAG 50mpk treatment, and MSU/PLAG 250mpk treatment.

FIGS. 37A-37D illustrate assessment of radiation-induced lung injury in control, radiation treated, radiation/PLAG 50mpk, and radiation/PLAG 250mpk BALB/C. FIG. 37A illustrates an experimental scheme for the evaluation of PLAG's therapeutic efficacy on radiation-treated lung injury. FIG. 37B illustrates lungs of control, radiated, radiation/PLAG 50mpk treated, and radiation/PLAG 250mpk treated mice. FIG. 37C illustrates H&E stained lung tissues of control, radiation treated, radiation/PLAG 50mpk treated, and radiation/PLAG 250mpk treated mice. FIG. 37D illustrates enlarged H&E stained lung tissues of control, radiation treated, radiation/PLAG 50mpk treated, and radiation/PLAG 250mpk treated mice.

FIGS. 38A-38C illustrate assessment of skin erythema injury in γ-ray radiated, and γ-ray radiation/PLAG treated BALB/c mice. FIG. 38A illustrates an experimental scheme for the evaluation of PLAG's therapeutic efficacy on radiation-treated skin erythema injury. FIG. 38B illustrates the feet and tails of radiated and radiation/PLAG treated mice. FIG. 38C illustrates tails of radiated and radiation/PLAG treated female and male mice.

FIGS. 39A and 39B illustrate the survival rate of γ-ray radiated, and γ-ray radiation/PLAG treated BALB/c mice. FIG. 39A illustrates an experimental scheme for the evaluation of PLAG's therapeutic efficacy on the survival rate of radiation-treated mice. FIG. 39B illustrates the survival rate of radiation-treated and radiation/PLAG treated mice over 30 days after radiation.

FIGS. 40A and 40B illustrate dose-dependency of PLAG on the survival rate of BALB/c mice. FIG. 40A illustrates an experimental scheme for the evaluation of PLAG's dose-dependent therapeutic efficacy on the survival rate of radiation-treated mice. FIG. 40B illustrates the survival rate of radiated, radiation/PLAG10mpk treated, radiation/PLAG 50mpk treated and radiation/PLAG 250mpk treated mice over 30 days after radiation.

FIGS. 41A and 41B illustrate the effects of PLAG on the body weight of BALB/c mice. FIG. 41A illustrates normalized body weight of radiated, radiation/PLAG10mpk treated, radiation/PLAG 50mpk treated and radiation/PLAG 250mpk treated mice over 30 days after radiation. FIG. 41B illustrates the percentage of radiated, radiation/PLAG10mpk treated, radiation/PLAG 50mpk treated and radiation/PLAG 250mpk treated mice whose body weight loss is higher than 10% and 20% respectively over 30 days after radiation.

FIGS. 42A and 42B illustrate the assessment of gemcitabine-induced CXCL2 and CXCL8 in control (non-treated), gemcitabine treated, and gemcitabine/PLAG treated male BALB/c mice. FIG. 42A illustrates RT-PCR assessment and relative MIP-2 expression of control mice, tumor-bearing mice, and gemcitabine treated tumor-bearing mice. FIG. 42B illustrates RT-PCR assessment and relative CXCL8 expression of control mice, gemcitabine-treated mice, gemcitabine treated mice with antagonists SCH202676, gallein, U73122 and rottlerin. FIG. 42C illustrates RT-PCR assessment and relative MIP-2 expression of control, gemcitabine treated, gemcitabine/PLAG1mpk treated, gemcitabine/PLAG10mpk treated and gemcitabine/PLAG 100mpk treated cells.

FIGS. 43A-43E illustrates the assessment of gemcitabine-induced ROS production in control (non-treated), gemcitabine treated, and gemcitabine/PLAG treated BMDMs and THP-1 cells. FIG. 43A illustrates flow cytometry data using chloromethyl derivative of 2′, 7′-dichlorodihydrofluorescein diacetate (CM-H2DCFDA) that is an indicator of ROS production. FIG. 43B illustrates relative intracellular ROS of control, gemcitabine treated, gemcitabine/PLAG 1mpk treated, gemcitabine/PLAG 10mpk treated, and gemcitabine/PLAG 100mpk treated cells in BMDM and THP-1 cells. FIG. 43C illustrates confocal microscopy of ROS production of control, gemcitabine treated, and gemcitabine/PLAG 1mpk treated cells in BMDM and THP-1 cells. FIG. 43D illustrates confocal microscopy of Ras-related C3 botulinum toxin substrate 1 (Rac1) membrane translocation of control, gemcitabine treated, and gemcitabine/PLAG treated cells in BMDM and THP-1 cells. FIG. 43E illustrates western blot analysis of cytosolic and membrane expressions of Rac1, Na/K-Adenosinetriphosphatase (ATPase), and α-tubulin in the gemcitabine treated cells (top), cytosolic and membrane expressions of Rac1, Na/K-ATPase, and α-tubulin of control in gemcitabine treated or gemcitabine/PLAG treated cells (middle), and phosphorylation of P47phox of control, gemcitabine treated, and gemcitabine/PLAG treated cells.

FIGS. 44A and 44B illustrate assessment of gemcitabine-induced phosphorylation of ROS dependent signal molecules in control (non-treated), gemcitabine treated, and gemcitabine/PLAG or DPI treated THP-1 cells. FIG. 44A illustrates phosphorylation of ERK (p-ERK), p38 MAPK (p-p38 MARK) and JNK (p-JNK) analyzed by western blot in control, gemcitabine-treated and gemcitabine/PLAG treated THP-1 cells. FIG. 44B illustrates phosphorylation of ERK (p-ERK), p38 MAPK (p-p38 MARK) and INK (P-JNK) analyzed by western blot in control, gemcitabine-treated and gemcitabine/diphenyleneiodonium (DPI) treated THP-1 cells.

FIGS. 45A-45G illustrates the assessment of gemcitabine-induced neutrophil extravasation in control (non-treated), gemcitabine treated, and gemcitabine/PLAG treated male BALB/c mice. FIG. 45A illustrates a population of circulating neutrophils in control, gemcitabine-treated and gemcitabine/PLAG treated cells analyzed by flow cytometry. FIG. 45B illustrates a population of Ly6G+/CD11b+ cells in control, gemcitabine-treated and gemcitabine/PLAG treated cells. FIG. 45C illustrates flow cytometry data illustrating the inhibition of gemcitabine treated L-selectin expression by PLAG (left) and fluorescence intensity illustrating the inhibition of gemcitabine treated lymphocyte function-associated antigen 1 (LFA-1) expression by PLAG (right). FIG. 45D illustrates gemcitabine-treated migration of circulating neutrophils from blood into the peritoneal cavity in tumor-bearing mice. FIG. 45E illustrates gemcitabine-treated migration of circulating neutrophils from blood into the peritoneal cavity in normal mice. FIG. 45F illustrates the count of circulating neutrophils from blood in control, gemcitabine-treated, gemcitabine/PLAG 50mpk treated, and gemcitabine/PLAG 250mpk treated mice. FIG. 45G illustrates PLAG's modulation of gemcitabine-treated migration of circulating neutrophils from blood into the peritoneal cavity in normal mice.

FIGS. 46A-46C illustrate assessment of 5-FU-induced neutropenia and reduction of monocyte in 5-FU treated, 5FU/PLAG 125 mpk treated, 5FU/PLAG 250 mpk treated male BALB/c mice. FIG. 46A illustrates an experimental scheme for the evaluation of PLAG's therapeutic efficacy on Fluorouracil (5-FU) treated neutropenia and the reduction of monocyte in mice. FIG. 46B illustrates neutrophil counts in 5-FU treated mice, 5-FU/PLAG 125 mpk, and 5-FU/PLAG 250 mpk over 15 days. FIG. 46C illustrates monocyte counts in 5-FU treated mice, 5-FU/PLAG 125 mpk, and 5-FU/PLAG 250 mpk over 15 days.

FIGS. 47A-47D illustrate assessment of chemotherapy-induced neutropenia in control (Gemcitabine/Erolobtinib) and Gemcitabine/Erolobtinib/PLAG treated human patients. FIG. 47A illustrates a table illustrating a control group (gemcitabine+erlotinib) and EC-18 treated gorup (gemcitabine+erlotinib+EC-18). FIG. 47B illustrates experimental scheme for the evaluation of PLAG's therapeutic efficacy on the incidence of neutropenia in patients. FIG. 47C illustrates relative absolute neutrophil count ANC after each of three cycles (gemcitabine+erlotinib+EC-18, gemcitabine+erlotinib). FIG. 47D illustrates incidence of neutropenia in control group (gemcitabine+erlotinib) and EC-18 treated group (gemcitabine+erlotinib+EC-18).

FIGS. 48A and 48B illustrate chemo-radiation induced oral mucositis (CRIOM) in control (non-treated), chemo-radiation treated, and chemo-radiation/PLAG treated BALB/c mice. FIG. 48A illustrates an experimental scheme for the evaluation of PLAG's therapeutic efficacy on chemoradiation treated oral mucositis (CRIOM). FIG. 48B illustrates tongues of control, radiation/chemotherapy/PBS treated and radiation/chemotherapy/PLAG treated mice.

FIGS. 49A and 49B illustrate assessment of chemo-radiation and scratch induced oral mucositis in radiation/chemotherapy/PBS and radiation/chemotherapy/PLAG treated mice. FIG. 49A illustrates the survival rate of radiation/chemotherapy/PBS treated and radiation/chemotherapy/PLAG treated mice over 18 days. FIG. 49B illustrates the tongues of radiation/chemotherapy/PBS treated and radiation/chemotherapy/PLAG treated mice.

FIGS. 50A and 50B illustrate the assessment of chemoradiation and PAK induced oral mucositis in PAK/chemotherapy/radiation/PBS treated and PAK/chemotherapy/radiation/PLAG treated male BALB/c mice. FIG. 50A illustrates the survival rate of PAK/chemotherapy/radiation/PBS treated and PAK/chemotherapy/radiation/PLAG treated mice over 18 days. FIG. 50B illustrates tongues of PAK/chemotherapy/radiation/PBS treated (upper row) and PAK/chemotherapy/radiation/PLAG treated mice (lower row).

FIGS. 51A-51E illustrate establishment of a chemoradiation-induced oral mucositis mouse model. FIG. 51A shows that on Day 0, the mice were divided into different groups. The mice then received 100 mg/kg 5-FU intraperitoneally and 20 Gy X-radiation to the head and neck region. Phosphate-buffered saline (PBS) or PLAG was administered orally each day until Day 9. FIG. 51B shows that changes in body weight were recorded each day and compared between groups. Data are shown as mean±SEM (#p<0.05, ***p<0.001, ###p<0.001 vs. Day 0). FIG. 51C shows that mice were sacrificed on Day 9, and their harvested tongues were stained with toluidine blue. FIG. 51D shows that tongues from each treatment group were stained with H&E. Scale bar=201 μm. FIG. 51E shows Histopathologic grading was determined for each treatment group.

FIGS. 52A-52G illustrate that 1-Palmitoyl-2-linoleoyl-3-acetyl-rac-glycerol (PLAG) attenuated chemoradiation-induced oral mucositis. FIG. 52S shows that ChemoRT (100 mg/kg 5-FU and 20 Gy X-radiation) was administered to the mice, with or without the addition of 100 mg/kg or 250 mg/kg PLAG. Body weight was recorded daily. Data are shown as mean±SEM (**p<0.01, ***p<0.001 vs. Day 0). FIG. 52B shows that on Day 9, mice were sacrificed, and the harvested tongues were stained with toluidine blue. FIG. 52C shows that ulcer size was measured using ImageJ, and the ratio of ulcer area/total area was expressed as a percentage. FIG. 52D shows tongues from each treatment group were stained with H&E. FIG. 52E shows that hHistopathologic grading was determined for each treatment group. Scale bar=201 μm. FIG. 52F shows that oral mucosa epithelial thickness was measured at 20 randomly selected sites in tissue slides and compared between groups. FIG. 52G shows that the experiment was repeated with ChemoRT and ChemoRT+PLAG 250 mg/kg-treated groups, and the harvested tongues were stained with toluidine blue for comparison. Data represent mean±SEM. Significant differences between groups with p<0.05 are marked with different letters (a, b and c).

FIGS. 53A-53E illustrate that 1-Palmitoyl-2-linoleoyl-3-acetyl-rac-glycerol (PLAG) ameliorated proinflammatory cytokine release and neutrophil infiltration. FIG. 53A shows that samples obtained from control, ChemoRT (100 mg/kg 5-FU+20 Gy) and ChemoRT+PLAG 250 mg/kg group mice on Day 9 were used to detect serum levels of the proinflammatory cytokines MIP-2 and IL-6. FIG. 53B shows that tongue extracts were used to detect MIP-2 and IL-6 levels. FIG. 53C shows that expression of MIP-2 (CXCL2) in tongue tissues was examined at the transcriptional level using RT-PCR. Relative expression was compared between groups. FIG. 53D shows that IL-6 mRNA expression was detected using RT-PCR, and relative expression was compared between groups. FIG. 53E shows that iImmunohistochemistry was performed with the neutrophil-specific antibody NIMP-R14. The ChemoRT group displayed neutrophil infiltration in the epithelium, whereas the PLAG co-treated group did not exhibit this infiltration. Neutrophils are stained brown. Scale bar=201 μm. Data are shown as mean±SEM (*/#p<0.05, **/##p<0.01, ***/##p<0.001).

FIGS. 54A-54B illustrate that release of DAMPs was reduced by PLAG. FIG. 54A shows that levels of DAMPs in the serum from control, ChemoRT (100 mg/kg 5-FU+20 Gy) and ChemoRT+PLAG 250 mg/kg group mice were examined by Western blotting. HMGB1 and Hsp90 were detected in the serum samples obtained on Day 9. Ponceau S staining of membrane proteins was used to demonstrate comparable protein loading. FIG. 54B shows that HMGB1 localization was observed by immunohistochemistry. Cytoplasmic HMGB1 was positively stained in the ChemoRT group. Nuclei are stained blue; HMGB1 is stained brown. Scale bars=201 μm (upper panels) and 40.1 μm (lower panels).

FIGS. 55A-55C illustrate that PLAG downregulated necroptosis signalling in tongues with chemoradiation-induced oral mucositis. FIG. 55A shows that protein levels of the necroptosis markers RIPK1, RIPK3 and MLKL were detected by Western blotting in tongue lysates from control, ChemoRT (100 mg/kg 5-FU+20 Gy) and ChemoRT+PLAG 250 mg/kg groups. FIG. 55B shows that band densities of phosphorylated RIPK1 (P-RIPK1), RIPK3 (P-RIPK3) and MLKL (P-MLKL) were compared to band densities of total RIPK1, RIPK3 and MLKL using ImageJ. FIG. 55C shows that P-MLKL was visualized by immunohistochemistry. P-MLKL is stained brown. Scale bar=201 μm. Data are shown as mean±SEM (*p<0.05, ***p<0.001 vs. ChemoRT using Student's t test).

FIG. 56 illustrates proposed schematic for the pathogenesis of chemoradiation-induced oral mucositis and the role of PLAG. Mice underwent intraperitoneal injection of 5-FU and head and neck X-irradiation. Chemoradiotherapy induced higher than normal levels of proinflammatory cytokines and DAMPs in the oral mucosa and serum. Accordingly, neutrophil infiltration in the oral epithelium was observed, and necroptosis signalling was activated in the tongues. By contrast, PLAG-treated mice had reduced DAMPs and cytokine levels by Day 9, which were similar to those of control mice who did not undergo chemoradiation. Furthermore, activation of the necroptosis signalling pathway (RIPK1, RIPK3 and MLKL axis) was reduced by PLAG treatment, protecting oral mucosa tissues from chemoradiation-induced damage.

### DETAILED DESCRIPTION OF THE INVENTION

A more detailed description of the invention will be made by reference to the attached drawings, which are intended for a better understanding of the present invention and will not limit the present invention.

Other than in the operating examples and unless otherwise stated, all numbers expressing quantities of ingredients and/or reaction conditions are to be understood as being modified in all instances by the term “about.”

Where the following terms are used in this specification, they are used as defined below.

The terms “comprising,” “having,” and “including” are used in their open, non-limiting sense.

The terms “a” and “the” are understood to encompass the plural as well as the singular.

As used herein, the expression “at least one” means one or more and thus includes individual components as well as mixtures/combinations.

As used herein, “pathogen” in the context of this invention refers to an organism that is capable of causing disease, for example, viruses and bacteria. Preferably, the pathogenic organism is a bacterium as most known pathogen-derived adjuvants are from bacteria.

As used herein, the term “treatment” or “treating” encompasses prophylaxis, reduction, amelioration or elimination of the condition to be treated, for example, suppression or delay of onset of inflammation by the administration of the pharmaceutical composition of the present invention (sometimes referred to as prevention), as well as improving inflammation or changing symptoms of inflammation to more beneficial states.

Monoacetyl Diacylglycerol

In one aspect, provided herein are compositions including compounds for modulating inflammatory response. In some embodiments, compositions of the present invention for modulating an inflammatory response include glycerol derivatives having one acetyl group and two acyl groups. In one embodiment, the two acylglycerol groups are identical. In another embodiment, the two acylglycerol groups are not identical.

For example, the glycerol derivative is a compound of the following Formula I:

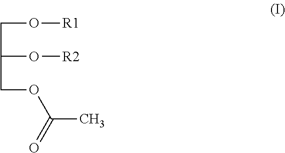

wherein R1 and R2 are independently a fatty acid residue of any number of carbon atoms. R1 and R2 may or may not be identical.

In some embodiments, R1 and R2 are independently a fatty acid residue having 14 to 22 carbon atoms. In some embodiments, the glycerol derivatives of Formula I, are herein referred as monoacetyl diacylglycerols (MDAG). Fatty acid residue, as used herein, refers to the acyl moiety resulting from the formation of an ester bond by the reaction of fatty acid and an alcohol. Non-limiting examples of R1 and R2 thus include palmitoyl, oleoyl, linoleoyl, linolenoyl, stearoyl, myristoyl, and arachidonoyl. In a preferred embodiment, a pair of R1 and R2 (R1/R2) comprises oleoyl/palmitoyl, palmitoyl/oleoyl, palmitoyl/linoleoyl, palmitoyl/linolenoyl, palmitoyl/arachidonoyl, palmitoyl/stearoyl, palmitoyl/palmitoyl, oleoyl/stearoyl, linoleoyl/palmitoyl, linoleoyl/stearoyl, stearoyl/linoleoyl, stearoyl/oleoyl, myristoyl/linoleoyl, and myristoyl/oleoyl. In the optical activity, the monoacetyl diacylglycerol (MADG) derivatives of Formula 1 can be (R)-form, (S)-form or a racemic mixture, and may include their stereoisomers. In some embodiments, when the R1 and/or R2 substituents are unsaturated fatty acid residues, one or more of the double bonds may have the cis configuration. In some embodiments, when the R1 and/or R2 substituents are unsaturated fatty acid residues, at least one of the double bond(s) may have the cis configuration. In some embodiments, when the R1 and/or R2 substituents are unsaturated fatty acid residues, at least one of the double bond(s) may have the trans configuration.

In some embodiments, the monoacetyl diacylglycerol (MADG) is a compound of the following Formula II:

The compound of Formula II is 1-palmitoyl-2-linoleoyl-3-acetylglycerol, herein

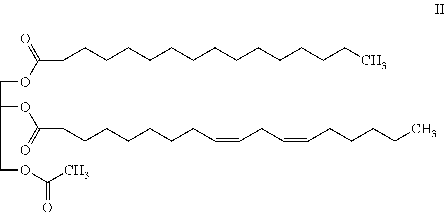 [SMILES: CCCCC/C=C\C/C=C\CCCCCCCC(=O)OC(COC(C)=O)COC(=O)CCCCCCCCCCCCCCC]

referred as “PLAG.” R1 and R2 of the compound of Formula II are palmitoyl and linoleoyl, respectively. The 2-carbon on the glycerol moiety is chiral. PLAG is generally provided as a racemate.

A pharmaceutical composition comprising one or more monoacetyl diacylglycerols may consist of only substantially pure monoacetyl diacylglycerol derivatives of Formula 1 or may include active components (monoacetyl diacylglycerol derivatives of Formula 1) and conventional pharmaceutically acceptable carriers, excipients, diluents, or combinations thereof. The amount of monoacetyl diacylglycerol in the pharmaceutical composition can be widely varied without specific limitation, and is specifically about 0.0001 to 100 weight %, about 0.001 to 95 weight %, about 0.01 to 90 weight %, about 0.1 to 85 weight %, about 1 to 80 weight %, about 5 to 75 weight %, about 10 to 70 weight %, about 15 to 65 weight %, about 20 to 60 weight %, about 25 to 55 weight %, about 30 to 50 weight %, or about 35 to 45 weight % with respect to the total amount of the composition. The pharmaceutical composition may be formulated into solid, liquid, gel or suspension form for oral or non-oral administration, for example, tablet, bolus, powder, granule, capsule such as hard or soft gelatin capsule, emulsion, suspension, syrup, emulsifiable concentrate, sterilized aqueous solution, non-aqueous solution, freeze-dried formulation, or suppository. In formulating the composition, conventional excipients or diluents such as filler, bulking agent, binder, wetting agent, disintegrating agent, and surfactant can be used. The solid formulation for oral administration includes tablet, bolus, powder, granule, and capsule, and the solid formulation can be prepared by mixing one or more of the active components and at least one excipient such as starch, calcium carbonate, sucrose, lactose, and gelatin. Besides the excipient, a lubricant such as magnesium stearate and talc can also be used. The liquid formulation for oral administration includes emulsion, suspension, and syrup, and may include one or more conventional diluents such as water and liquid paraffin or may include various excipients such as wetting agent, sweetening agent, flavoring agent, and preserving agent. The formulation for non-oral administration includes sterilized aqueous solution, non-aqueous solution, freeze-dried formulation, and suppository, and solvent for such a solution may include propylene glycol, polyethylene glycol, vegetable oil such as olive oil, and ester for syringe injection such as ethyl oleate. Base materials of the suppository may include one or more selected from triglycerides (e.g. WITEPSOL®), polyethylene glycol (PEG) (e.g., macrogol), fatty acid (e.g., stearic acid, palmitic acid, or TWEEN®61), cacao butter (e.g., LAURIN®) and glycerogelatine.

The effective amount of the composition of the present invention can be varied according to the condition and weight of the patient, the severity of the disease, formulation type of drug, administration route and period of treatment. An effective total amount of administration per 1 day can be determined by a physician, and is generally about 0.05 to 200 mg/kg, about 0.1 to 150 mg/kg, about 1 to 100 mg/kg, about 10 to 50 mg/kg, about 0.05 to 200 mg/kg, or about 0.05 to 200 mg/kg. Extrapolating from in vivo experiments in animals and in vitro experiments in cells, the preferable total administration amount per day is determined to be about 0.1 to 100 mg/kg, about 1 to 90 mg/kg, about 10 to 80 mg/kg, about 20 to 70 mg/kg, about 30 to 60 mg/kg, or about 40 to 50 mg/kg for an adult human. For example, the total amount of 50 mg/kg can be administered once a day or can be administered in divided doses twice, three, or four times daily.

For example, in some embodiments, provided is a novel pharmaceutical composition in a unit dosage form for oral or non-oral administration. In some embodiments, the form is a tablet. In some embodiments, the form is a bolus. In another embodiment, the form is a powder. In some embodiments, the form is a granule. In some embodiments, the form is a capsule, such as hard or soft gelatin capsule. In some embodiments, the form is an emulsion. In some embodiments, the form is a suspension. In some embodiments, the form is a syrup. In some embodiments, the form is an emulsifiable concentrate. In some embodiments, the form is a sterilized aqueous solution. In some embodiments, the form is a non-aqueous solution. In some embodiments, the form is a freeze-dried formulation. In some embodiments, the form is a suppository.

For oral administration, in some embodiments, the form includes from about 100 to about 4000 mg, from about 200 to about 3900 mg, from about 300 to about 3800 mg, from about 400 to about 3700 mg, from about 500 to about 3600 mg, from about 600 to about 3500 mg, from about 700 to about 3400 mg, from about 800 to about 3300 mg, from about 900 to about 3200 mg, from about 1000 to about 3100 mg, from about 1100 to about 3000 mg, from about 1200 to about 2900 mg, from about 1300 to about 2800 mg, from about 1400 to about 2700 mg, from about 1500 to about 2600 mg, from about 1600 to about 2500 mg, from about 1700 to about 2400 mg, from about 1800 to about 2300 mg, from about 1900 to about 2200 mg, or from about 2000 to about 2100 mg of PLAG drug substance, free of other triglycerides. For oral administration, in some embodiments, the form includes from about 100 to about 4000 mg, from about 200 to about 3900 mg, from about 300 to about 3800 mg, from about 400 to about 3700 mg, from about 500 to about 3600 mg, from about 600 to about 3500 mg, from about 700 to about 3400 mg, from about 800 to about 3300 mg, from about 900 to about 3200 mg, from about 1000 to about 3100 mg, from about 1100 to about 3000 mg, from about 1200 to about 2900 mg, from about 1300 to about 2800 mg, from about 1400 to about 2700 mg, from about 1500 to about 2600 mg, from about 1600 to about 2500 mg, from about 1700 to about 2400 mg, from about 1800 to about 2300 mg, from about 1900 to about 2200 mg, or from about 2000 to about 2100 mg of PLAG drug substance, substantially free of other triglycerides. In some embodiments, the form further includes from about 0.1 to 500 mg, from about 50 to 450 mg, from about 100 to 400 mg, from about 150 to 350 mg, or from about 200 to 300 mg of pharmaceutically acceptable antioxidants. In one embodiment, the pharmaceutically acceptable antioxidants include a tocopherol compound. In some embodiments, the tocopherol compound is α-tocopherol.

The composition of the present invention can be administered once or twice a day, at a daily dosage of from about 100 to about 5000 mg, from about 200 to about 4900 mg, from about 300 to about 4800 mg, from about 400 to about 4700 mg, from about 500 to about 4600 mg, from about 600 to about 4500 mg, from about 700 to about 4400 mg, from about 800 to about 4300 mg, from about 900 to about 4200 mg, from about 1000 to about 4100 mg, from about 1100 to about 4000 mg, from about 1200 to about 3900 mg, from about 1300 to about 3800 mg, from about 1400 to about 3700 mg, from about 1500 to about 3600 mg, from about 1600 to about 3500 mg, from about 1700 to about 3400 mg, from about 1800 to about 3300 mg, from about 1900 to about 3200 mg, from about 2000 to about 3100 mg, from about 2100 to about 3000 mg, from about 2200 to about 2900 mg, from about 2300 to about 2800 mg, from about 2400 to about 2700 mg, or from about 2500 to about 2600 mg. For example, the composition of the present invention can be administered at a daily dosage of 1000 mg/day by administering 500 mg in the morning and 500 mg in the evening. In some embodiments, the composition of the present invention further includes from about 0.1 to 200 mg, from about 20 to 180 mg, from about 40 to 160 mg, from about 60 to 140 mg, or from about 80 to 120 mg of pharmaceutically acceptable diluent or carrier.

The composition of the present invention can be administered to any subject that requires modulation of an inflammatory response. In some embodiments, the subject is a cell. The cell may be a eukaryotic cell. The eukaryotic cell may be a mammalian cell. The eukaryotic cell may be a human cell. The eukaryotic cell may be a phagocyte. The eukaryotic cell may be selected from the group consisting of a macrophage, a neutrophil, a monocyte, a mast cell, a dendritic cell, a fibroblast, and an epithelial cell. The eukaryotic cell may be a non-human cell. The composition of the present invention can be further administered to not only humans, but also non-human animals (specifically, mammals), such as monkey, dog, cat, rabbit, guinea pig, rat, mouse, cow, sheep, pig, and goat. The composition of the present invention can be administered by conventional various methods. The methods include oral administration, rectum administration, intravenous (i.v.) injection, intramuscular (i.m.) injection, subcutaneous (s.c.) injection, or cerebrovascular injection. As monoacetyl diacylglycerols are orally active, they are suitably administered orally, for example in the form of a gelatin capsule, or the form of a health functional food, that is, a food which contains an effective amount of a monoacetyl diacylglycerol compound of Formulae I or II.

In some embodiments, the compound of Formula II is administered in the form of a soft gelatin capsule. The soft gelatin capsule includes from about 100 to about 4000 mg, from about 200 to about 3900 mg, from about 300 to about 3800 mg, from about 400 to about 3700 mg, from about 500 to about 3600 mg, from about 600 to about 3500 mg, from about 700 to about 3400 mg, from about 800 to about 3300 mg, from about 900 to about 3200 mg, from about 1000 to about 3100 mg, from about 1100 to about 3000 mg, from about 1200 to about 2900 mg, from about 1300 to about 2800 mg, from about 1400 to about 2700 mg, from about 1500 to about 2600 mg, from about 1600 to about 2500 mg, from about 1700 to about 2400 mg, from about 1800 to about 2300 mg, from about 1900 to about 2200 mg, or from about 2000 to about 2100 mg of Formula II in combination or association with from about 0.1 to 200 mg, from about 20 to 180 mg, from about 40 to 160 mg, from about 60 to 140 mg, or about 80 to 120 mg of pharmaceutically acceptable diluent or carrier. For example, the soft gelatin capsule includes 250 mg of the Compound of Formula II in combination or association with approximately 50 mg of a pharmaceutically acceptable diluent or carrier, an edible oil, e.g., a vegetable oil, e.g., olive oil. In some embodiments, the soft gelatin capsule further includes from about 0.1 to 500 mg, from about 50 to 450 mg, from about 100 to 400 mg, from about 150 to 350 mg, or from about 200 to 300 mg of pharmaceutically acceptable antioxidants.

A chemical synthetic method for the preparation of monoacetyl diacylglycerol compounds is shown, for example, in Korean Registered Patents No. 10-0789323 and No. 10-1278874, the contents of which are incorporated herein by reference.

Monoacetyl diacylglycerol (MADG) compounds, including PLAG, have been found to be specifically recognized by, sassociated with, or bound to a pattern recognition receptor such as a scavenger receptor-A. The receptor colocalizes with another pattern nrecognition receptor recognizing, associating with, or binding to Pathogen-Associated Molecular Pattern (PAMP) or Damage-Associated Molecular Pattern (DAMP) molecules to accelerate intracellular trafficking of the colocalized receptors, thereby enhancing removal of PAMP and DAMP molecules.

Pathogen-Associated Molecular Pattern (PAMP)

Pathogen-Associated Molecular Pattern (PAMP) molecules are a diverse set of microbial molecules that share a number of different general patterns, or structures, that alert immune cells to destroy intruding pathogens. Pathogen-Associated Molecular Pattern (PAMP) molecules can initiate and perpetuate the infectious pathogen inflammatory response. Alternatively, Pathogen-Associated Molecular Pattern (PAMP) molecules can be any molecule recognized by, associated with, or bound to a Pathogen-Associated Molecular Pattern (PAMP) receptor. A Pathogen-Associated Molecular Pattern (PAMP) receptor is one kind of pattern recognition receptors (PRRs), nucleotide-binding oligomerization domain-like receptors. It will be understood by a person skilled in the art that the various PAMP molecules and PAMP receptors are well established in the art.

In some embodiments, Pathogen-Associated Molecular Pattern (PAMP) molecules include a bacterial PAMP, a viral PAMP, a fungal PAMP, a protozoan PAMP or a combination thereof. PAMP may further include debris, toxins, nucleic acid variants associated with bacteria, viruses, fungi, or protozoa. In some embodiments, bacterial PAMP includes one or more PAMPs from gram-positive bacteria, gram-negative bacteria, mycobacteria, intracellular bacteria, flagellated bacteria, or mycoplasma, and/or molecules derived from there. In some embodiments, the viral PAMP includes one or more selected from PAMPs from measles virus, HSV, cytomegalovirus, RSV, influenza A virus, HCV, RSV, picornavirus, or norovirus, and/or molecules derived from there. In some embodiments, fungal PAMP includes one or more PAMPS from Candida albicans, Aspergillus fumigatus, Cryptococcus neoformans, and Pneumocystis jirovecii, and/or molecules derived therefrom. In some embodiments, protozoan PAMP includes one or more of glycosylphosphatidylinositol (GPI) anchors, unmethylated DNA, Toxoplasma gondii (T. gondii), and molecules derived therefrom. The bacterial PAMP is, for example, a lipopolysaccharide (LPS), a bacterial peptide (e.g. flagellin, microtubule elongation factors), a peptidoglycan, a lipoteichoic acid, a mannose, a lipoprotein, a diacyl lipoprotein and a nuclic acid such as a bacterial DNA or RNA. The viral PAMP is, for example, a nucleic acid, such as a viral DNA or RNA.

Damage-Associated Molecular Pattern (DAMP)

Damage-Associated Molecular Pattern (DAMP) molecules, also known as danger-associated molecular pattern molecules, as used herein are multifunctional modulators of the immune system. Damage-Associated Molecular Pattern (DAMP) molecules can initiate and perpetuate immune responses in an inflammatory response. When released outside the cell or exposed on the surface of the cell following tissue injury, they may move from a reducing to an oxidizing milieu, which can result in their denaturation. Damage-Associated Molecular Pattern (DAMP) molecules can be alternatively defined to be any molecule recognized by, associated with, or bound to a DAMP receptor. A DAMP receptor is one kind of pattern recognition receptors (PRRs), nucleotide-binding oligomerization domain-like receptors. It will be understood by a person skilled in the art that the various DAMP molecules and DAMP receptors are well established in the art.

In some embodiments, Damage-Associated Molecular Pattern (DAMP) molecules include, but are not limited to, one or more selected from DNA, RNA, purine metabolites such as nucleotides (e.g., ATP), nucleosides (e.g., adenosine), uric acid, heparin sulfate, nanoparticles, asbestos, aluminum compositions such as aluminum salts, beta-amyloid, silica, cholesterol crystals, hemozoin, calcium pyrophosphate dehydrate, monosodium urate (MSU), imiquimod (IMQ), intracellular proteins such as high mobility group box 1 (HMGB1), S100 molecules, F-actin, LDH, mono and polysaccharides and the like.

Pattern Recognition Receptor (PRR)

A pattern recognition receptor (PRR) mediates the initial response to infection. The intracellular signaling cascades triggered by these PRRs lead to the transcriptional expression of inflammatory mediators that coordinate the elimination of pathogens and infected cells. PRR includes, but is not limited to, a damage-associated molecular pattern receptor, a pathogen-associated molecular pattern receptor, a toll-like receptor (TLR), a C-type lectin receptor (CLR), a G protein-coupled receptor (GPCR), a scavenger receptor or a combination thereof.

Toll-like receptors (TLRs) are a class of proteins that play a key role in the innate immune system. TLRs are single, membrane-spanning, non-catalytic receptors usually expressed on sentinel cells such as macrophages and dendritic cells that recognize structurally conserved molecules derived from microbes. Once these microbes have reached physical barriers, such as the skin or intestinal tract mucosa, they are recognized by TLRs, which activate immune cell responses. For example, TLR is expressed on the membranes of leukocytes including dendritic cells, macrophages, natural killer cells, cells of the adaptive immunity (T and B lymphocytes) and non-immune cells (epithelial and endothelial cells, and fibroblasts).

The toll-like receptor (TLR) includes, but is not limited to, TLR1, TLR2, TLR3, TLR4, TLR5, TLR6, TLR7, TLR8, TLR9, TLR10, TLR11, TLR12, and TLR13. TLR1 recognizes bacterial lipoprotein and peptidoglycans. TLR2 recognizes bacterial peptidoglycans. TLR3 recognizes double-stranded RNA. TLR4 recognizes lipopolysaccharides. TLR5 recognizes bacterial flagella. TLR6 recognizes bacterial lipoprotein. TLR7 recognizes single-stranded RNA, bacterial, and viral. TLR8 recognizes single-stranded RNA, bacterial and viral, phagocytized bacterial RNA. TLR9 recognizes CpG DNA. TLR10 recognizes triacylated lipopeptides. TLR11 recognizes profilin from Toxoplasma gondii, also possibly uropathogenic bacteria. TLR12 recognizes profilin from Toxoplasma gondii. TLR13 recognizes bacterial ribosomal RNA.

The C-type lectin receptor (CLR) is divided into mannose receptors and asialoglycoprotein receptors. The mannose receptor (MR) is a pattern recognition receptor (PRR) primarily present on the surface of macrophages and dendritic cells. MR is selected from the group consisting of MRC1, C-type mannose receptor 1, CLEC13D, CD206, MMR, C-type mannose receptor 2, urokinase-type plasminogen activator receptor-associated protein, and CD280. The asialoglycoprotein receptor is selected from the group consisting of macrophage galactose-type lectin (MGL), CD209, CDSIGN, CLEC4L, DC-SIGN, DC-SIGN1, CD209 molecule, langerin, CD207, CLEC4K, CD207 molecule, myeloid DAP 12-associating lectin (MDL)-1, CLEC5A, DC-associated C-type lectin 1, dectin-1, CLEC7A, CLECSF12, BGR, CANDF4, CLEC2, CLEC1B, CLEC2B, DNGR-1, CLEC9A, Dectin-2, CLEC4N, CLEC6A, CLECSF10, Nkcl, CLECSF8, CLEC4D, CLEC6, MCL, MPCL, CLEC4C, BDCA2, dectin-3, mincle, CLEC4E, CLECSF9, MICL, CLEC12A, CLL-1, CLL1, DCAL-2, and KLRL1.

The G protein-coupled receptor includes one or more of rhodopsin-like G Protein-coupled receptors, secretin family receptor proteins, metabotropic glutamate receptors, fungal mating pheromone receptors, cyclic AMP receptors, and frizzled/smoothened G Protein-coupled receptors. Alternatively, the G Protein-coupled receptor (GPCR) includes one or more purinergic G Protein-coupled receptors, for example, a P2Y1, P2Y2, P2Y4, P2Y6, P2Y11, P2Y12, P2Y13, and P2Y14 receptors.

Scavenger Receptor (SR)

Among various pattern recognition receptors, scavenger receptors include a diverse group of receptors that are categorized into class A, class B and class C. Class A is mainly expressed in the macrophage and is composed of cytosol domain, a transmembrane domain, spacer domain, alpha-helical coiled-coil domain, collagen-like domain and cysteine-rich domain. Class B has two transmembrane regions. Class C is a transmembrane protein whose N-terminus is located extracellularly. Exemplary Class A receptors include one or more selected from the group consisting of MSR1, CD204, SCARA1, SR-A, SRA, phSR1, phSR2, macrophage scavenger receptor 1, SR-AI, SR-AII, SR-AIII, MARCO, SCARA2, macrophage receptor with collagenous structure, SR-A6, SCARA3, SCARA4, COLEC12, SCARA5. Exemplary Class B receptors include one or more selected from the group consisting of SCARB1, CD36L1, CLA-1, CLA1, HDLQTL6, SR-BI, SRB1, scavenger receptor class B member 1, SCARB2, AMRF, CD36L2, EPM4, HLGP85, LGP85, LIMP-2, LIMPII, SR-BII, scavenger receptor class B member 2, CD36, BDPLT10, CHDS7, FAT, GP3B, GP4, GPIV, PASIV, SCARB3 and CD36 molecule.

Among various scavenger receptors, class A scavenger receptors (SR-A) are a subclass of PRRs expressed on macrophages that bind to numerous foreign substances including bacterial cell wall components and modified low-density lipoproteins (LDLs) in the blood for the removal of these non-self or altered-self molecules by the processes of recognition, internalization, adhesion and signal transduction. SR-A was originally defined by its ability to accumulate lipids in the cytoplasm of macrophages, and many studies focused on the role of this receptor in atherosclerosis. Subsequent researches on this receptor have revealed that SR-A also plays an important role in innate immune activity by synergistically collaborating with other PRRs. For example, SR-A forms complexes with Toll-like receptor 4 (TLR4) for the efficient engulfment and elimination of gram-negative bacteria like P. aeruginosa and Escherichia coli (E. coli), and also forms complexes with Toll-like receptor 2 (TLR2) for effective phagocytosis of gram-positive bacteria such as Staphylococcus aureus (S. aureus).

PLAG Modulated Phagocytosis

The innate immune system is the first line of defense against invading pathogens. Phagocytes play an important role in eliminating the microorganisms via phagocytosis.

During phagocytosis, a pathogen-associated molecular pattern (PAMP) molecule is recognized by, binds to, or associates with a PAMP receptor located on the surface of a cell. The cell engulfs the PAMP molecule to form an internal component known as a phagosome. It is distinct from other forms of endocytosis, like pinocytosis that involves the internalization of extracellular liquids. Monoacetyl diacylglycerol (MADG), when administered, has been surprisingly found to modulate phagocytosis by accelerating removal of pathogen-associated molecular pattern (PAMP) molecules from an extracellular space and thus effectively reduce inflammation. Monoacetyl diacylglycerol (MADG) is first recognized by, binds to, or associates with a scavenger receptor located on the cell membrane. The PAMP receptor associated with the PAMP molecules and the scavenger receptor associated with the MADG co-localizes on the membrane surface of the cell to be internalized in the cell. MADG has been found to accelerate the intracellular trafficking of the colocalized receptors such as a purinergic G protein-coupled receptor (GPCR), PAMP molecules and MADG. PLAG has been also found to modulate GPCR related MAPK pathway by modulating the phosphorylation of extracellular signal-regulated kinases (ERK), c-Jun N-terminal kinases (JNK), p38 mitogen-activated protein kinases (P38MAPK). The internalization forms a vesicle called phagosome containing the receptors, PAMP molecules and MADG in the cell. PAMP molecules in the phagosome stimulate the generation of ROS and lysosomal activity to eliminate or destroy the PAMP molecules therein.

Reactive oxygen species (ROS) are chemically reactive chemical species containing oxygen. Exemplary ROSs include peroxides, superoxide, hydroxyl radical, singlet oxygen, and alpha-oxygen. For example, NOX activation (e.g., NOX2 activation) has a close relationship with the production of phagosomal ROSs. When NOX2 is activated, cytosolic subunit proteins such as p47phox, p67phox, and Rac1 are translocated to the membrane on which membrane-bound subunit proteins, gp91phox and p22phox, are localized. PLAG accelerates membrane localization of the cytosolic subunit proteins by membrane fractionation. PLAG promotes the membrane localization of p47phox, p67phox and Rac1 at early time points, and released the proteins back to the cytosol at late time points. Along with ROS production, stimulated lysozyme activity is also a critical process for successful bacterial killing.

During phagocytosis, lysosomal vesicles are fused with the bacteria-containing phagosome and destroy the bacteria with hydrolytic enzymes in acidic conditions. MADG has been found to accelerate PAMP-induced recruitment of p47phox enzyme, intracellular ROS production, and intracellular lysosomal activity in the same time frame, thereby engulfing and clearing PAMP molecules earlier and returning to homeostatic status faster. This phenomenon has been also found in mice immunocompromised by the treatment of chemotherapeutic agents such as AC regimen (e.g., 50 mg/kg of cyclophosphamide and 2.5 mg of doxorubicin). MADG significantly accelerates bacterial clearance in immunocompromised mice.

In particular, the accelerated production of the ROS attenuates a signaling to phosphorylate interferon regulatory factor (IRF) and a mixed lineage kinase domain-like pseudokinase (MLKL) by a receptor-interacting protein kinase (RIPK) including, but not limited to, RIPK1 and RIPK3. PLAG has been found to dose-dependently modulate the phosphorylation of RIPK1, RIPK3, and MLKL. Less phosphorylated IRF leads to a decreased expression of one or more cytokines, one or more chemokines, or a combination thereof. Cytokine is a category of small proteins that are important in cell signaling. Chemokine, or chemotactic cytokines, is a family of small cytokines, or signaling proteins secreted by cells, which are able to induce directed chemotaxis in nearby responsive cells. For example, cells that are attracted by chemokines follow a signal of increasing chemokine concentration towards the source of the chemokine. Thus, less phosphorylated MLKL leads to a decreased expression of DAMP molecules. Therefore, during phagocytosis, MADG collectively accelerates removal of PAMP molecules and decreases in expression of cytokines, chemokines, DAMP molecules, or combinations thereof, which leads to less neutrophil recruitment or extravasation to inflammation site and less severe DAMP-induced inflammation, thereby modulating pathogen-derived inflammation.

In some embodiments, an extracellular space of the cell includes an increased level of pathogen-associated molecular pattern (PAMP) molecules derivated from invading pathogens. The increased level of PAMP molecules can be induced by a bacterial infection, viral infection, or a combination thereof. The increased level of PAMP molecules can be also linked to infectious diseases including, but not limited to, pneumonia and acute lung injury (ALI).

In some embodiments, the cell is a eukaryotic cell. In some embodiments, the eukaryotic cell is a mammalian cell. In some embodiments, the eukaryotic cell is a human cell. In some embodiments, the eukaryotic cell is a phagocyte. The phagocyte may include, but not be limited to, a macrophage, a neutrophil, a monocyte, a mast cell, a dendritic cell, a fibroblast, and an epithelial cell. In some embodiments, the monocyte is a bone marrow-derived monocyte (BMDM).

In some embodiments, pathogen-associated molecular pattern (PAMP) molecule is a bacterial PAMP, a viral PAMP, a fungal PAMP, a protozoan PAMP or a combination thereof. PAMP may further include debris, toxins, nucleic acid variants associated with bacteria or viruses. The bacterial PAMP includes, for example, one or more selected from lipopolysaccharide (LPS), a bacterial peptide (e.g., flagellin, microtubule elongation factors), peptidoglycan, a lipoteichoic acid, a mannose, a lipoprotein, a diacyl lipoprotein and a nucleic acid such as a bacterial DNA or RNA. The viral PAMP includes, for example, one or more nucleic acids such as viral DNA or RNA. The fungal PAMP is includes, for example, one or more selected from Candida albicans, Aspergillus fumigatus, Cryptococcus neoformans, and Pneumocystis jirovecii, and molecules derived therefrom. The protozoan PAMP include, for example, one or more selected from glycosylphosphatidylinositol (GPI) anchors, unmethylated DNA, Toxoplasma gondii (T. gondii), and molecules derived therefrom.

In some embodiments, a scavenger receptor is, but not limited to, selected from the group consisting of MSR1, CD204, SCARA1, SR-A, SRA, phSR1, phSR2, macrophage scavenger receptor 1, SR-AI, SR-AII, SR-AIII, MARCO, SCARA2, macrophage receptor with collagenous structure, SR-A6, SCARA3, SCARA4, COLEC12, SCARA5, SCARB1, CD36L1, CLA-1, CLA1, HDLQTL6, SR-BI, SRB1, scavenger receptor class B member 1, SCARB2, AMRF, CD36L2, EPM4, HLGP85, LGP85, LIMP-2, LIMPII, SR-BII, scavenger receptor class B member 2, CD36, BDPLT10, CHDS7, FAT, GP3B, GP4, GPIV, PASIV, and SCARB3.

In some embodiments, PLAG, a monoacetyl diacylglycerol, associates with a scavenger receptor located (SR-A) on a membrane surface. A PAMP molecule associates with a toll-like receptor 4 (TLR4) located on a membrane surface. PLAG is first recognized by, binds to, or associates with the SR-A located on the cell membrane. The TLR4 associated with the PAMP molecules and the scavenger receptor associated with the PLAG colocalize on the membrane surface of the cell to be internalized in the cell. PLAG has been found to accelerate the intracellular trafficking of the colocalized receptors, PAMP molecules, and PLAG. The internalization forms a vesicle called phagosome containing the receptors, PAMP molecules and PLAG in the cell. PAMP molecules in the phagosome stimulate the generation of ROS and lysosomal activity to eliminate or destroy the PAMP molecules therein. Along with ROS production, stimulated lysozyme activity is also a critical process for successful bacterial killing. During phagocytosis, lysosomal vesicles are fused with the bacteria-containing phagosome and destroy the bacteria with hydrolytic enzymes in acidic conditions. PLAG has been found to accelerate PAMP-induced ROS production and lysosomal activity.

PLAG has been also found to be in the form of a vesicle/micelle. PLAG is a lipid molecule in which palmitic and linoleic acid are esterified to the first and second site of the glycerol backbone, and acetyl acid to the third site. This lipid molecule can be used as a structural component for the formation of micelle monolayer. PLAG forms micelles and contacts with the cells in the form of micelles by interacting with LPL, GPIHBP-1, and SR-A. Knockdown of LPL, GPIHBP-1, and SR-A abrogates the effect of PLAG on the advanced phagocytosis and ROS production. The knockdown of either LPL or GPIHBP-1 showed not only impaired phagocytosis of PAMP molecules but also ineffective bacterial killing capacity. PLAG enhances neither phagocytosis nor bacterial killing in LPL or GPIHBP-1 silenced cells. The knockdown of SR-A does not affect intracellular bacterial loads but does not show the enhanced phagocytosis by PLAG that is observed in intact cells. In SR-A silenced cells, PLAG does not increase PAMP molecules-induced intracellular ROS. Therefore, PLAG enhances bacterial internalization and ROS production by interacting with SR-A.

Among a variety of monoacetyl diacylglycerols, PLAG shows the best phagocytosis of PAMP molecules such as PAK. The acetyl group in PLAG has been shown critical in bacterial internalization and phagocytosis because PLH, a diacylglycerol without an acetyl group, shows little effect on bacterial internalization. Further, PLAG shows the most advanced phagocytosis among other monoacetyl diacylglycerols LLAG, MLAG, PLAG, SLAG, or ALAG and PLH. This confirms that PLAG is biologically the most optimal molecule to eliminate PAMP molecules through phagocytosis.

PLAG Modulated Necroptosis

Necrosis has been considered an accidental cell death and not set to determined pathways or cellular regulation. Necrotic cell death is defined by an increase in cell volume, swelling of organelles, plasma membrane rupture, and eventual leakage of intracellular components. Current research is determining that necrosis is not just a series of unregulated, uncontrollable processes but may in fact be a series of programmed necrosis or necroptosis. Recent findings have shown that after inhibition of caspase activity in genetic models, or by using specific caspase inhibitors, an apoptosis-independent type of necroptosis can occur. Thus, necroptosis is currently considered as a specialized biochemical pathway of programmed necrosis.

Necroptosis has been shown to be mediated by the kinase activity of receptor-interacting proteins 1 and 3 (RIP1 and RIP3). Phosphorylation-driven assembly of the RIP1-RIP3 necrosis complex seems to regulate necroptosis. For the activation of necroptosis, the kinase activity of both RIP1 and RIP3 is required.

Like phagocytosis, a damage-associated molecular pattern (DAMP) molecule is recognized by, binds to, or associates with a DAMP receptor located on a cell surface. Monoacetyl diacylglycerol (MADG), when administered, has been surprisingly found to modulate necroptosis by accelerating removal of DAMP molecules from an extracellular space and thus effectively reduce inflammation.

Monoacetyl diacylglycerol (MADG) is first recognized by, binds to, or associates with a scavenger receptor located on the cell membrane. The DAMP receptor associated with the DAMP molecules and the scavenger receptor associated with the MADG co-localizes on the membrane surface of the cell to be internalized in the cell. MADG has been found to accelerate the intracellular trafficking of the colocalized receptors, DAMP molecules and MADG, thereby engulfing and clearing DAMP molecules earlier and returning to homeostatic status faster. The internalization forms a vesicle called endosome containing the receptors, DAMP molecules and MADG in the cell. DAMP molecules in the endosome stimulate the generation of ROS and lysosomal activity to eliminate or destroy the DAMP molecules therein. Along with ROS production, stimulated lysozyme activity is also a critical process for successful DAMP removal. During necroptosis, lysosomal vesicles are fused with the DAMP molecules containing endosomes and destroy the DAMP molecules with hydrolytic enzymes in acidic conditions. MADG has been found to accelerate DAMP-induced ROS production and lysosomal activity. In particular, the accelerated production of the ROS attenuates a signaling to phosphorylate interferon regulatory factor (IRF) and a mixed lineage kinase domain-like pseudokinase (MLKL) by a receptor-interacting protein kinase (RIPK). Less phosphorylated MLKL leads to a decreased expression of one or more cytokines, one or more chemokines, or a combination thereof. Therefore, during necroptosis, MADG collectively accelerates removal of DAMP molecules and decreases in the expression of cytokines, chemokines, or DAMP molecules, which leads to less neutrophil recruitment to inflammation site and less severe DAMP-induced inflammation, thereby modulating inflammation.

In some embodiments, PLAG, a monoacetyl diacylglycerol, associates with a scavenger receptor located (SR-A) on a membrane surface. A DAMP molecule associates with a P2Y6 receptor located on a membrane surface. PLAG is first recognized by, binds to, or associates with the SR-A located on the cell membrane. The P2Y6 associated with the DAMP molecules and the scavenger receptor associated with the PLAG colocalize on the membrane surface of the cell to be internalized in the cell. PLAG has been found to accelerate the intracellular trafficking of the co-localized receptors, DAMP molecules, and PLAG. The internalization forms a vesicle called endosome containing the receptors, DAMP molecules and PLAG in the cell. DAMP molecules in the endosome stimulate the generation of ROS and lysosomal activity to eliminate or destroy the DAMP molecules therein. Along with ROS production, stimulated lysozyme activity is also a critical process for successful DAMP removal. During necroptosis, lysosomal vesicles are fused with the DAMP molecules containing phagosome and destroy the DAMP molecules with hydrolytic enzymes in acidic conditions. PLAG has been found to accelerate DAMP-induced ROS production and lysosomal activity.

In some embodiments, an extracellular space of the cell includes an increased level of damage-associated molecular pattern (DAMP) molecules. The increased level of DAMP molecules can be induced by inflammation. The increased level of DAMP molecules can be also linked to infectious diseases including, but not limited to, chemotherapy-induced neutropenia (CIN), chemo-radiation induced oral mucositis (CRIOM), skin erythema, and psoriasis. PLAG is capable of modulating the clearance of DAMP molecules and neutropenia caused by neutrophil extravasation induced by chemotherapy, radiation, scratch, or a combination thereof and preventing tissue damages resulting from DAMP molecules. PLAG has been found to dose-dependently significantly increase survival rate and maintain the body weight of mice treated by chemotherapy, radiation, or a combination thereof.

In some embodiments, the cytokines or chemokines include, but are not limited to, CXCL2, CXCL8, and IL-6.

PLAG Modulated NETosis

During NETosis, neutrophils recruited by one or more cytokines, one or more chemokines, or a combination thereof form a neutrophil extracellular traps (NETs)-like structure to remove one or more pathogen-associated molecular pattern (PAMP) molecules, one or more damage-associated molecular pattern (DAMP) molecules, or a combination thereof. Neutrophils play a key role in the innate immune system, as these cells are the first leukocytes to migrate to regions of acute inflammation. Neutrophils cross the blood vessel endothelium into infected tissue and eliminate invading pathogens via multiple killing mechanisms, including phagocytosis, degranulation, and neutrophil extracellular traps (NETs). Notably, neutrophils secrete numerous cytokines and chemokines that influence other immune cells and are thus key regulators of inflammation.

Neutrophil extracellular traps (NETs) that contain large web-like structures of decondensed chromatin attached with histones and intracellular components, including neutrophil elastase (NE), myeloperoxidase (MPO), high mobility group protein B1 (HMGB1), and proteinase 3 (PR3), are extruded into the extracellular space. In particular, the histones and intracellular components have a high affinity for DNA and are capable of removing or destroying PAMP and DAMP molecules. Therefore, neutrophils are critical immune cells in host defense against infections, such as bacterial and fungal infection. Monoacetyl diacylglycerol (MADG), when administered, has been found to modulate NETosis by promoting a formation of NETs-like structure. MADG contributes to activation of phospholipase C (PLC) to cleave a phosphatidylinositol biphosphate (PIP2) into an inositol trisphosphate (IP3) and a diacylglycerol (DAG) in the neutrophil. IP3 increases the level of intracellular calcium ions in the neutrophil. The increased concentration of intracellular calcium ions, in turn, activates a protein arginine deiminase (PAD) in the neutrophil. In particular, the nuclear translocation of PAD4 is essential for histone citrullination during calcium-dependent NETosis. During histone citrullination, PAD4 decondensed a chromatin in the neutrophil to form a neutrophil extracellular traps (NETs)-like structure by releasing intracellular components such as neutrophil elastase, myeloperoxidase, and nucleotides outside the neutrophil. Therefore, during NETosis, MADG promotes a formation of NETs-like structure from neutrophils to remove PAMP molecules, DAMP molecules or combinations thereof, thereby contributing to modulation of inflammation.

In some embodiments, PLAG, when administered, modulates NETosis by promoting a formation of NETs-like structure. PLAG contributes to the activation of phospholipase C (PLC) to cleave a phosphatidylinositol biphosphate (PIP2) into an inositol trisphosphate (IP3) and a diacylglycerol (DAG) in the neutrophil. The IP3 increases the level of intracellular calcium ions in the neutrophil. The increased concentration of intracellular calcium ions activates a protein arginine deiminase (PAD) in the neutrophil. In particular, the nuclear translocation of PAD4 is essential for histone citrullination during calcium-dependent NETosis. During histone citrullination, PAD4 decondensed a chromatin in the neutrophil to form a neutrophil extracellular traps (nets)-like structure by releasing intracellular components such as neutrophil elastase, myeloperoxidase, and nucleotide ouside the neutrophil. PLAG has been found to promote the NETosis of PAMP molecule-introduced bone marrow-derived cells and BALF derived cells by increasing intracellular calcium and histone citrullination. The formation of the neutrophil extracellular traps (NETs)-like structure eventually results in neutrophil death. Thus, the apoptotic neutrophils, considered damage-associated molecular patterns (DAMPs), need to be removed during the following process called efferocytosis.

PLAG Modulated Efferocytosis

During efferocytosis, a cell recognizes “find me” signals comprising nucleotides or chemokines secreted by the apoptotic cell or the necrotic cell including a dead neutrophil releases. Improper clearance of apoptotic neutrophils often causes an unnecessary and exaggerated immune response and subsequent chronic inflammation. Thus, proper efferocytosis of apoptotic neutrophils is crucial for tissue homeostasis, because its dysregulation can lead to unwanted inflammation, autoimmunity, and an exacerbated immune response. Monoacetyl diacylglycerol has been found to enhance the efferocytosis of apoptotic neutrophil. MDAG promotes macrophage mobility, thereby increasing the apoptotic neutrophil efferocytotic effect of macrophages. The modulation of macrophage mobility was confirmed to be due to faster polarization of the cytoskeleton induced by the acceleration of P2Y2 migration to the non-lipid-raft domain induced by MDAG. This repositioning of P2Y2 enables the polarization of the cytoskeleton by the association of the receptor with cytoskeletal proteins such as α-tubulin and actin to improve the mobility of macrophages. It was also found that the formation of vesicular, chylomicron-like structures by MDAG was a prerequisite for the induction of this macrophage activity, as none of these effects were seen when the vesicle receptor GPIHBP1 was absent. Taken together, these results suggest that during efferocytosis, MDAG collectively modulates the mobility of macrophage, thereby modulating inflammation induced by apoptotic neutrophils.

Nucleotides secreted from dead cells are key factors for macrophage recruitment. The nucleotides may include, but not be limited to, one or more adenosine triphosphate (ATP), adenosine diphosphate (ADP), uridine triphosphate (UTP) and uridine diphosphate (UDP). The nucleotides are recognized by, associated with or bound to the P2Y2 receptor, which is a crucial step for the timely clearance of apoptotic neutrophils.

In some embodiments, the cell is a eukaryotic cell. In some embodiments, the eukaryotic cell is a human cell. In some embodiments, the human cell is a phagocyte. The phagocyte may include, but not be limited to, a macrophage, a neutrophil, a monocyte, a mast cell, a dendritic cell, a fibroblast, and an epithelial cell. In one embodiment, the monocyte is a bone marrow-derived monocyte.

In some embodiments, a compound comprising a monoacetyl diacylglycerol is administered to a cell. The administration modulates phagocytosis by the cell. The modulation of phagocytosis by the cell includes an acceleration of the removal of an apoptotic cell or a necrotic cell from extracellular space. The process is termed efferocytosis.

In some embodiments, PLAG has been found to enhance efferocytosis of apoptotic neutrophil in a dose-dependent manner. PLAG promotes macrophage mobility, thereby increasing the apoptotic neutrophil efferocytotic effect of macrophages. The modulation of macrophage mobility was confirmed to be due to faster polarization of the cytoskeleton induced by the acceleration of P2Y2 migration to the non-lipid-raft domain induced by PLAG. This repositioning of P2Y2 enables the polarization of the cytoskeleton by the association of the receptor with cytoskeletal proteins such as α-tubulin and actin to improve the mobility of macrophages. It was also found that the formation of vesicular, chylomicron-like structures by PLAG was a prerequisite for the induction of this macrophage activity, as none of these effects were seen when the vesicle receptor GPIHBP1 was absent.

MADG Uptake in the Body

MADG, in particular, PLAG, once administered, has been found to be delivered from intestinal lumen through enterocytes to lymphatic vessels. MADG is digested in the intestinal lumen and absorbed into intestinal epithelial cells in the form of 2-monoacyl glyceride (2MAG) and fatty acid. MADG is reconstituted with the aid of monoacylglycerol acyltransferases (MGAT) and diacylglycerol acyltransferases (DGAT) and assembled as chylomicrons. The chylomicrons are absorbed in peripheral tissues with the aid of lipoprotein lipase (LPL). PLAG dose-dependently improves lipid metabolism especially in hepatic steatosis by promoting the uptake of the chylomicrons to peripheral tissues. This reduceschylomicron delivered to the liver and alleviates hepatic steatosis. Synthesized chylomicrons in enterocyte move through the lymphatic vessel and toward cisterna chyli, subclavian vein and join into a blood vessel. PLAG uptake in the cisterna chyli with about 50% of absorptive efficacy has been confirmed. More PLAG is detected in the lymphatic vessel than blood vessels indicating that absorbed PLAG transfers to tissues through the lymphatic vessel, which is the same as the dietary lipid absorption pathway due to its structural similarity. Further, absorbed PLAG has been found to be excreted through expired air by the lung, and approximately 76% of PLAG administered is degraded and metabolized within 24 hours.

The following examples are provided for better understanding of this invention. However, the present invention is not limited by the examples.

### Example 1 PLAG Modulates LPS-Induced Endocytosis

Materials and Methods

Raw264.7 cells were divided into two groups: 1) LPS treated group and 2) LPS/PLAG treated group. For LPS treated group, cells were stimulated with LPS (100 ng/ml) for 0, 15, 30, 45, 60, 75, 90, 105, 120 minutes. For LPS/PLAG treatment, cells were pre-incubated with PLAG (100 μg/ml) for 1 hour and then stimulated with LPS (100 ng/ml) for 0, 15, 30, 45, 60, 75, 90, 105, 120 minutes. To detect TLR4/MD2 on the membrane surface, cells were fixed with 2% paraformaldehyde (Sigma-Aldrich) and were blocked with PBS containing 1% BSA (Gibco, Waltham, Mass., USA). They were incubated with rabbit anti-TLR4/MD2 antibody (Thermo) and Alexa488 conjugated anti-rabbit IgG (Invitrogen). For confocal microscopy analysis, cells were washed with PBS and mounted in DAPI-containing fluorescence microscopy mounting medium (Invitrogen). Samples were analyzed with a laser scanning confocal microscope (Carl Zeiss, Oberkochen, Germany).

Results

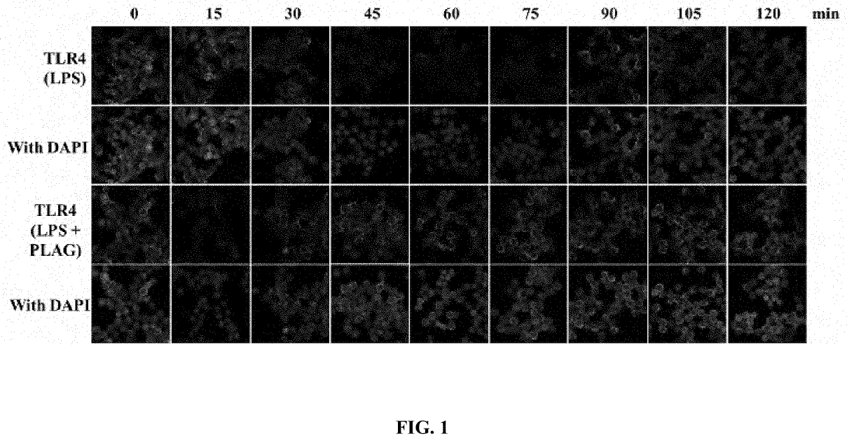

To investigate the effect of PLAG on internalization of the Lipopolysaccharide (LPS)/toll-like receptor 4 (TLR4) complex, TLR4/Lymphocyte antigen 96 (MD2) on the surface of LPS treated and LPS/1-palmitoyl-2-linoleoyl-3-acetyl-rac-glycerol (PLAG) treated RAW264.7 cells using anti-TLR4/MD2 antibody and Alexa488 conjugated anti-rabbit IgG secondary antibody was analyzed by confocal microscopy (FIG. 1). LPS, a gram-negative bacteria surface molecule, is well known as exotoxin and as a PAMP molecule. LPS is recognized by TLR4. LPS/PLAG treated Raw264.7 cells showed more rapid endocytosis of the LPS/TLR4 complex and earlier recovery of TLR4 on surface membranes than those treated with LPS alone. Specifically, the initiation of TLR4 internalization was observed 30 minutes after LPS treatment and after 15 minutes after LPS/PLAG treatment. Similarly, the return of the TLR4 receptor to the cell surface membrane occurred 120 minutes after LPS treatment and 60 minutes after LPS/PLAG treatment. These data show that PLAG accelerates the intracellular trafficking of the TLR4 receptor.

### Example 2 PLAG Modulates LPS-Induced ROS Production

Materials and Methods

Raw264.7 cells were divided into two groups: 1) LPS treated group and 2) LPS/PLAG treated group. For LPS treated group, cells were stimulated with LPS (100 ng/ml) for 0, 15, 30, 45, 60, 75, 90, 105, 120 minutes. For LPS/PLAG treatment, cells were pre-incubated with PLAG (100 ng/ml) for 1 hour and then stimulated with LPS (100 ng/ml) for 0, 15, 30, 45, 60, 75, 90, 105, 120 minutes. To detect intracellular ROS, cells were treated with FITC conjugated-CM-H2DCFDA (Invitrogen, Carlsbad, Calif., USA) for 30 minutes before LPS treatment for LPS treated group and before PLAG treatment for LPS/PLAG treated group. For confocal microscopy analysis, cells were washed with PBS and mounted in DAPI-containing fluorescence microscopy mounting medium (Invitrogen). Samples were analyzed with a laser scanning confocal microscope (Carl Zeiss, Oberkochen, Germany).

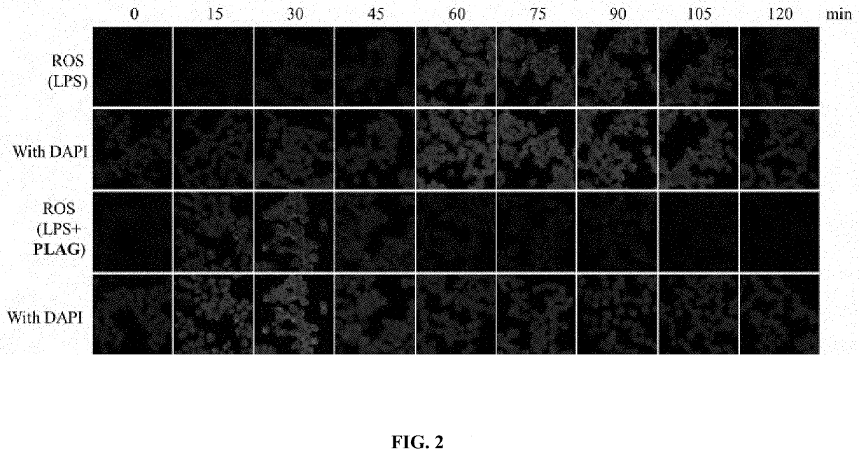

It was further investigated whether PLAG stimulates the generation of intracellular LPS-induced reactive oxygen species (ROS) (FIG. 2). It is well known that, in macrophages, internalized LPS spontaneously stimulates the generation of ROS, which function to eliminate or clear the source of intracellular LPS. This also activates signaling pathways leading to the production of numerous chemokines (mainly MIP-2) that recruit circulating neutrophils to the infection site. ROS generation is closely regulated by the nicotinamide adenosine dinucleotide phosphate (NADPH) oxidase system (Segal et al., 2000). ROS production initiated 60 minutes after LPS treatment and 15 minutes after LPS/PLAG treatment. Similarly, return to homeostatic levels of intracellular ROS occurred 120 minutes after LPS treatment and 60 minutes after LPS/PLAG treatment. These data, indicate that PLAG accelerates LPS-induced ROS production.

### Example 3 PLAG Modulates LPS-Induced Lysosomal Activity

Materials and Methods

Raw264.7 cells were divided into two groups: 1) LPS treated group and 2) LPS/PLAG treated group. For the LPS treated group, cells were treated with 100 μg/ml of DMSO (as solvent control) for 1 hour and treated with LPS (100 ng/ml) for 15, 30, 60, and 120 minutes. For LPS/PLAG treated group, cells were treated with PLAG (100 μg/ml) for 1 hour and treated with LPS (100 ng/ml) for 15, 30, 60, and 120 minutes. Cells were then fixed and stained using rat anti-TLR4/MD2 antibody with Alexa488-conjugated anti-rat IgG secondary antibody. These were analyzed by confocal microscopy. Raw264.7 cells stimulated under the same conditions were fixed, permeabilized, and stained with CM-H2DCFDA, the LYSO-ID® Lysosomal Detection Kit and rabbit anti-p47phox. Confocal microscopy was performed; all data shown represent one experiment performed in triplicate.

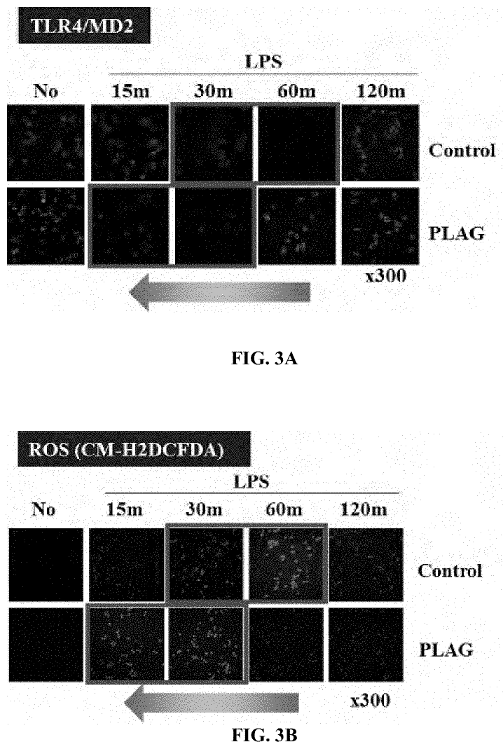

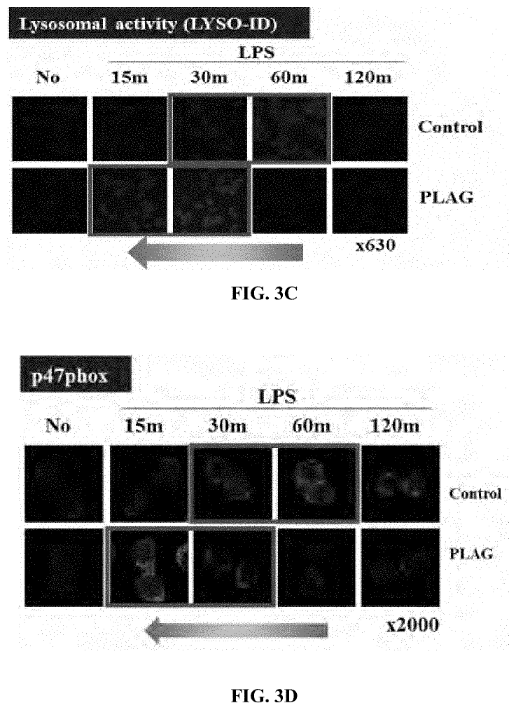

It was further investigated whether PLAG accelerates lysosomal activity as well in the presence of LPS (FIGS. 3A-3D). Here, it was found that in LPS-stimulated Raw264.7 cells, initiation of TLR4 internalization was observed 30 minutes after LPS treatment and 15 minutes after LPS/PLAG treatment. Similarly, the return of the TLR4 receptor to the cell surface membrane occurred 120 minutes after LPS treatment and 60 minutes after LPS/PLAG treatment (FIG. 3A).

Further, ROS production initiated 30 minutes after LPS treatment and 15 minutes after LPS/PLAG treatment. Similarly, return to homeostatic levels of intracellular ROS occurred 120 minutes after LPS treatment and 60 minutes after LPS/PLAG treatment (FIG. 3B). Lysosomal activity initiated 30 minutes after LPS treatment and 15 minutes after LPS/PLAG treatment. Similarly, return to homeostatic levels of lysosomal activity occurred 120 minutes after LPS treatment and 60 minutes after LPS/PLAG treatment (FIG. 3C). P47phox recruitment initiated 30 minutes after LPS treatment and 15 minutes after LPS/PLAG treatment. Similarly, return to homeostatic levels of p47phox occurred 120 minutes after LPS treatment and 60 minutes after LPS/PLAG treatment (FIG. 3D).

Thus, FIGS. 3A-3D reveals that PLAG accelerates the endocytosis of LPS/TLR4, the recruitment of p47phox enzyme, intracellular ROS production, and intracellular lysosomal activity in the same time frame, thereby clearing LPS earlier and faster.

### Example 4 PLAG Modulates LPS-Induced Acute Lung Injury (ALI)

Materials and Methods

Evans Blue Leakage Assay

Evans blue (50 mg/kg, Sigma-Aldrich) was diluted in PBS and injected intravenously into mice 30 minutes before sacrifice. After sacrifice, mice were perfused by right ventricle puncture with PBS, and lungs were photographed. Following drying at 56° C. for 48 hours, the lungs were weighed, and Evans blue dye was extracted in 500 μl of formamide (Sigma-Aldrich). The absorbance of these supernatants was measured by spectrophotometry (Molecular Devices, Sunnyvale, Calif., USA) at a wavelength of 620 nm. Evans blue concentrations were calculated as extracted Evans blue concentration (ng) divided by the dry lung tissue weight (mg) and compared to measurements from a standard curve.

Hematoxylin and Eosin Staining and Immunohistochemistry

Lung tissue specimens were fixed in 10% buffered formalin for 24 hours, embedded in paraffin, and sectioned at 4 μm. Tissue sections were stained with hematoxylin and eosin (H&E). For immunohistochemistry (IHC) analyses, 4-μm thick lung serial sections were cut and mounted on charged glass slides (Superfrost Plus; Fisher Scientific, Rochester, N.Y., USA). The sections were deparaffinized and then treated with 3% hydrogen peroxide in methanol to quench the endogenous peroxidase activity. Samples were then incubated with 1% bovine serum albumin (BSA; Gibco) to block non-specific binding. After blocking, sections were incubated with primary rat anti-neutrophil (NIMP-R14, Thermo Fisher Scientific Inc., Waltham, Mass., USA) antibody (1:100) or mouse anti-LPS (Abeam, Cambridge, UK) antibody (1:100) at 4° C. overnight. After washing, the slides were incubated with a 1:250 dilution of the secondary antibody, either horseradish peroxidase-conjugated goat-anti-rat IgG (Santa Cruz Biotechnology, Dallas, Tex., USA) or horseradish peroxidase-conjugated goat-anti-mouse IgG (Dako, Santa Clara, Calif., USA), at room temperature for 15 minutes. Images were observed under light microscopy (Olympus, Shinjuku, Tokyo, Japan).

Histological Scoring and Myeloperoxidase Activity Assay

Lung injury scores were measured by a blinded investigator using published criteria (Table 1 and Equation 1), which are based on neutrophil infiltration (in the alveolar or the interstitial space), hyaline membranes, proteinaceous debris filling the airspaces, and septal thickening (Matute-Bello et al., 2011). To measure myeloperoxidase (MPO) activity in ALI mice, lungs were isolated and homogenized with 0.1% IGEPAL® CA-630 (Sigma-Aldrich). After centrifugation for 30 minutes, MPO activity was determined using the Myeloperoxidase Activity Assay Kit (Abcam). Sample absorbance was measured using a microplate reader (Molecular Devices) at 410 nm.

TABLE 1
Lung injury scoring criteria (from Matute-bello et al.)

| | Score per field | | |
|---|---|---|---|
| Parameter | 0 | 1 | 2 |
| A. Neutrophils in the alveolar space | None | 1-5 | >5 |
| B. Neutrophils in the interstitial space | None | 1-5 | >5 |
| C. Hyaline membranes | None | 1 | >1 |
| D. Proteinaceous debris filling the airspaces | None | 1 | >1 |
| E. Alveolar septal thickening | <2x | 2x-4x | >4x |

$$The final score = \frac{[ ( 20 \times A ) + ( 14 \times B ) + ( 7 \times C ) + ( 7 \times D ) + ( 2 \times E ) ]}{Number of fields \times 100} . \qquad Equation 1$$

RT-PCR and Real-Time PCR

Total RNA was extracted using the Total RNA Extraction Solution (Favorgen, Taiwan), according to the manufacturer's instructions. The extracted RNA was used in reverse transcription reactions with oligo-dT primers and M-MLV RT reagents (Promega, Madison, Wis., USA), according to the manufacturer's instructions. For RT-PCR, the synthesized cDNA was mixed with 2×PCR Master Mix (Solgent, Daejeon, Republic of Korea) and 10 pmol specific PCR primer pair following the manufacturer's protocol. The primers were synthesized from Macrogen (Seoul, Republic of Korea; see Table 2 for primer sequences). Amplified products were separated on 1% agarose gels, stained with ethidium bromide, and photographed under UV illumination using a GelDoc (Bio-Rad Laboratories, Hercules, Calif., USA).

An SYBR Green kit (Applied Biosystems, Foster City, Calif., USA) was used for real-time PCR (qPCR) analysis of cDNA according to the manufacturer's instructions. Thermal cycling conditions were as follows: initial denaturation at 95° C. for 15 minutes, followed by 40 cycles of 95° C. for 30 seconds, 60° C. for 30 seconds, and 72° C. for 30 s. A melting step was performed by raising the temperature from 72° C. to 95° C. after the last cycle. Thermal cycling was conducted on a ViiA 7 Real-Time PCR System machine (Applied Biosystems). The target gene expression levels are shown as a ratio in comparison with GAPDH expression in the same sample by calculation of cycle threshold (Ct) value. The relative expression levels of target genes were calculated by the 2^{-ΔΔCT} relative quantification method. GAPDH was used as a control.

TABLE 2
Primers used for PCR

| | Sense primer | Antisense primer |
|---|---|---|
| MIP-2 | AGTGAACTGCGCTGTCAATG | CTTTGGTTCTTCCGTTGAGG |
| | (SEQ ID NO. 1) | (SEQ ID NO. 2) |
| S100A8 | ATGCCGTCTGAACTGGAGAA | TGCTACTCCTTGTGGCTGTC |
| | (SEQ ID NO. 3) | (SEQ ID NO. 4) |
| S100A9 | ATGGCCAACAAAGCACCTT | TTACTTCCCACAGCCTTTGC |
| | (SEQ ID NO. 5) | (SEQ ID NO. 6) |
| GAPDH | CCATCACCATCTTCCAGGAG | ACAGTCTTCTGGGTGGCAGT |
| | (SEQ ID NO. 7) | (SEQ ID NO. 8) |

Enzyme-Linked Immunosorbent Assay (ELISA)

The concentration of MIP-2 was measured using ELISA kits for MIP-2 (R&D Systems, Minneapolis, Minn., USA) according to the manufacturers' instructions. Cytokine levels were estimated by interpolation from a standard curve generated using an ELISA reader (Molecular Devices) at 450 nm.

Results

LPS can recruit immune cells into the lung alveolar compartment and promote the secretion of inflammatory mediators. Thus, LPS is commonly used to induce the development of ALI in a mouse model. Mice were divided into three separate groups (n=5 per group): control (non-treated), LPS-treated, and LPS/PLAG treated. LPS (25 mg/kg) was intranasally injected, and PLAG (250 mg/kg) was administered orally. All in vivo data were obtained from at least three independent experiments with five mice for each group. Data shown represent one experiment performed in triplicate (p<0.05).

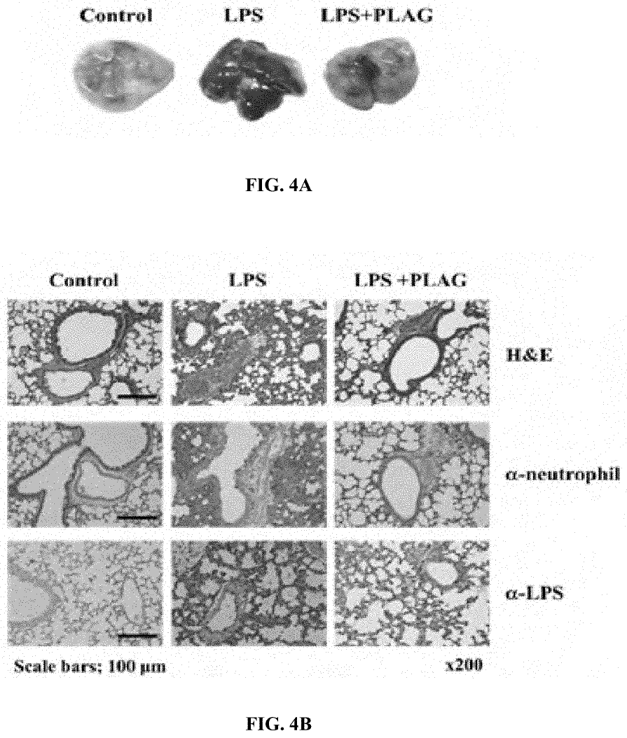

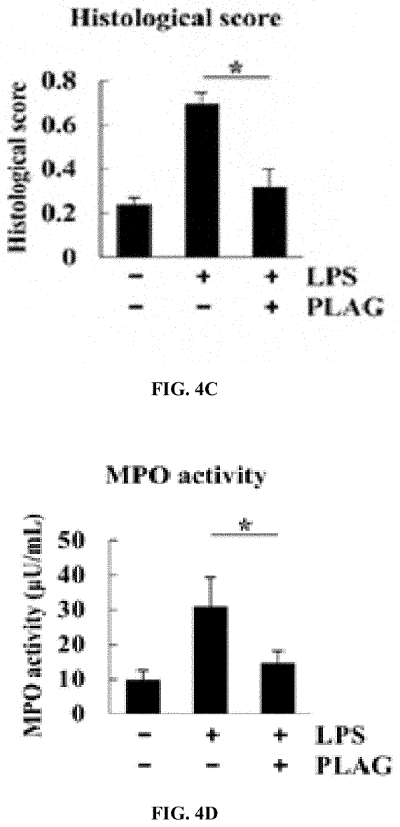

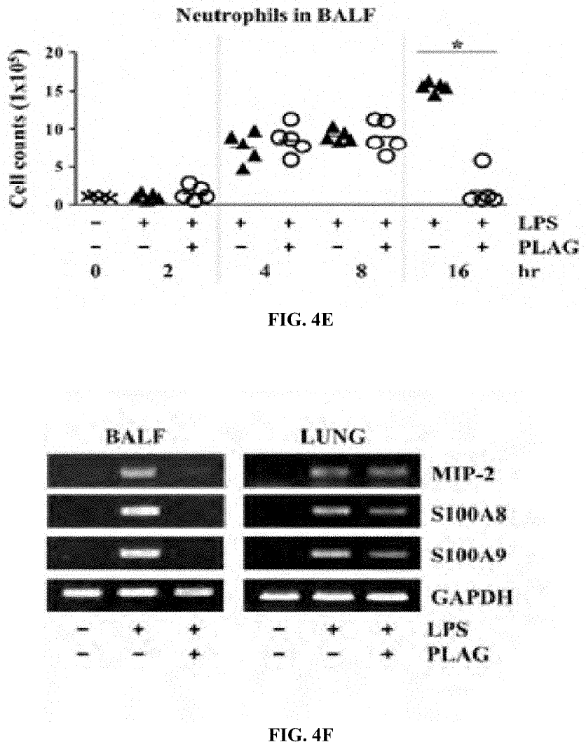

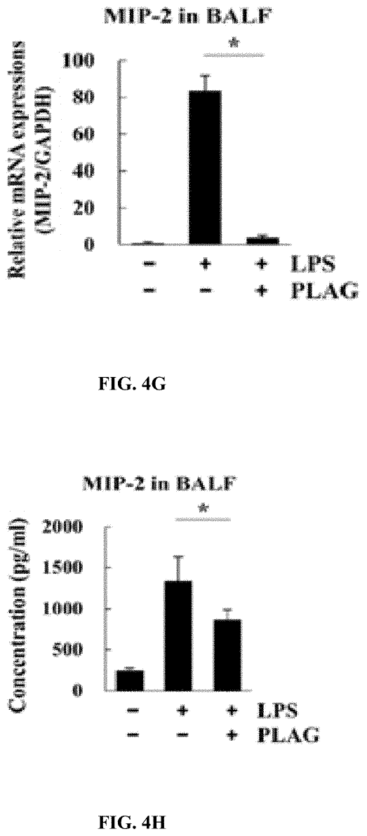

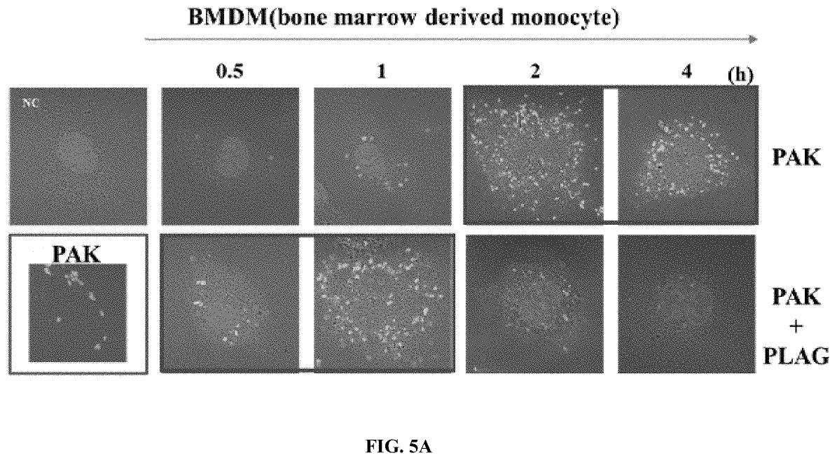

Contol (non-treated), LPS treated, and LPS/PLAG treated lungs of mice were stained with Evans blue dye (FIG. 4A). LPS treated lung tissues showed excessive leakage of albumin from blood vessels to the alveolar space, as demonstrated by increased Evans blue staining. Lungs from mice treated with LPS/PLAG, however, showed significantly decreased Evans blue-stained albumin. A high level of Evans blue staining is correlated with the vast extravasation of neutrophils into the alveolar space. Thus, PLAG mitigated LPS-induced extravasation of neutrophils into alveolar space.

To further examine the effect of PLAG on leukocyte cell infiltration into the lung alveolar compartment, hematoxylin and eosin (H&E) staining was applied to each group: control (non-treated), LPS-treated, and LPS/PLAG treated. Histological examination of lung tissues was performed 16 hours after LPS administration. Lung sections were stained with H&E, neutrophil, and LPS-specific antibodies (FIG. 4B).

Lung tissue specimens were fixed in 10% buffered formalin for 24 hours, embedded in paraffin, and sectioned at 4 μm. Tissue sections were stained with H&E. These data revealed that intranasal LPS administration induces extensive inflammatory cell infiltration into the lung tissue compared to control animals. However, LPS/PLAG treated mice exhibited a considerably reduced inflammatory cell infiltration into the alveolar space and displayed normal alveolar morphology.

Further, lung injury scoring of control (non-treated), LPS treated, and LPS/PLAG treated lungs was calculated (FIG. 4C). Lung injury scores were measured by a blinded investigator using published criteria (Table 1 and Equation 1), which are based on neutrophil infiltration (in the alveolar or the interstitial space), hyaline membranes, proteinaceous debris filling the airspaces, and septal thickening (Matute-Bello et al., 2011). MPO activity was examined for lungs from control, LPS, and LPS/PLAG-treated mice (FIG. 4D). An increase in MPO activity reflects neutrophil accumulation in the lungs. MPO activity of isolated lung tissue was found to substantially increase in LPS-treated mice but was significantly decreased in the LPS/PLAG treated mice, as compared to those treated with LPS alone. These data suggest that PLAG plays a protective role in ALI by blocking excessive neutrophil influx into the lung tissue.

Following LPS or LPS/PLAG stimulation for 2, 4, 8, and 16 hours, mice were sacrificed, and the number of neutrophils in bronchoalveolar lavage fluid (BALF) was counted using complete blood count (CBC) analysis (FIG. 4E). The bar represents the mean. LPS treatment is found to significantly increase neutrophil infiltration into BALF compared to the control. However, LPS/PLAG treated animals more rapidly return to homeostasis, showing baseline numbers of neutrophils in BALF by 16 hours post-treatment. PLAG treatment alone has no effect on neutrophil migration, and LPS/PLAG treatment does not alter neutrophil release from bone marrow or apoptosis. Thus, these data indicate that PLAG can specifically modulate excessive neutrophil infiltration into the lung.

To more precisely determine the role of PLAG in controlling excessive neutrophil infiltration into lung tissue in the ALI model, the expression of several inflammation-related molecules in BALF cells and lung-homogenized tissue after treatment with PLAG and/or LPS for 16 h was measured. It was found that mRNA expression levels of MIP-2 (CXCL2), the main factor involved in neutrophil migration, as well as S100A8 and S100A9, are increased in BALF cells from mice treated with LPS for 16 h compared to those from control animals (FIGS. 4F and 4G). Total RNA was extracted from BALF cells and homogenized lungs after LPS treatment and LPS/PLAG treatment and analyzed by reverse transcription RT-PCR (FIG. 4F) and real-time PCR (qPCR) (FIG. 4G). The gene expression increased by LPS, however, was significantly attenuated in mice treated with PLAG for 16 hours.

TABLE 2

| | Sense primer | Antisense primer |
|---|---|---|
| MIP-2 | AGTGAACTGCGCTGTCAATG | CTTTGGTTCTTCCGTTGAGG |
| | (SEQ ID NO. 1) | (SEQ ID NO. 2) |
| S100A8 | ATGCCGTCTGAACTGGAGAA | TGCTACTCCTTGTGGCTGTC |
| | (SEQ ID NO. 3) | (SEQ ID NO. 4) |
| S100A9 | ATGGCCAACAAAGCACCTT | TTACTTCCCACAGCCTTTGC |
| | (SEQ ID NO. 5) | (SEQ ID NO. 6) |
| GAPDH | CCATCACCATCTTCCAGGAG | ACAGTCTTCTGGGTGGCAGT |
| | (SEQ ID NO. 7) | (SEQ ID NO. 8) |

The concentration of secreted MIP-2 in BALF after LPS treatment and LPS/PLAG treatment were measured using an enzyme-linked immunosorbent assay (ELISA) (FIG. 4H).

### Example 5 PLAG Modulates PAK-Induced Bacteria Internalization in the Bone Marrow-Derived Macrophage (BMDM) or a Human Monocytic Cell Line (THP-1)

Materials and Methods

In Vitro Phagocytosis and Bacterial Killing Assay

For immunofluorescence-based measurement of phagocytosis and clearance, BMDMs were grown on glass coverslips in 24-well plates. The cells were infected with PAK (MOI, 50) for different time intervals and then were treated with 10 μg/ml gentamycin for 30 minutes to remove extracellular PAK attached to the cell surface. The infected cells were washed with ice-cold PBS several times, followed by fixing for 10 minutes at room temperature in methanol or 10% paraformaldehyde. The cells were stained with anti-Pseudomonas antibody (Abcam) and then were incubated with goat anti-rabbit IgG secondary antibody conjugated with Alexa Fluor 488. Slides were mounted using the mounting medium ProLong™ Gold antifade reagent with the DNA-binding blue dye DAPI (Thermo Scientific™) and were imaged with a confocal microscope (Zeiss LSM 800, Germany).

For CFU counting-based phagocytosis and bacterial killing assay, PAK was cultured at 37° C. overnight with continuous shaking and was resuspended in PBS. The BMDMs or THP-1 cells were incubated with PAK (MOI, 50) for different time intervals at 37° C. The cells were further cultured in the medium containing 10 μg/ml gentamycin for 30 minutes and then were lysed by 0.5% SDS. The diluted aliquots were spread on LB agar plates, and CFU was counted after incubation of the plates overnight at 37° C.

Results

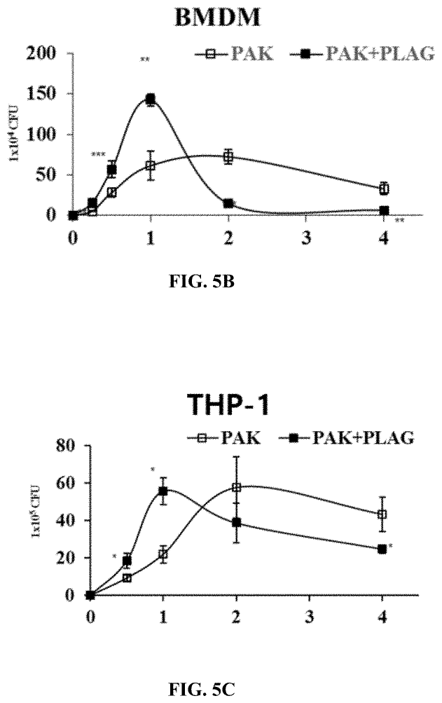

To evaluate whether PLAG accelerates phagocytosis of PAK by macrophages in vitro systems, bone marrow-derived macrophages (BMDMs) were pretreated with PLAG, and then infected with PAK (MOI, 50) for different lengths of time. Gentamycin (2 mg/ml) was treated to the cells for 30 minutes to remove extracellular bacteria. Immunofluorescence micrographs of BMDMs incubated with PAK confirmed that PLAG accelerated not only engulfment of bacteria, but also clearance of bacteria (FIG. 5A). This study shows accelerated phagocytosis by proving the effect of PLAG using live bacteria Pseudomonas aeruginosa K. PAK, gram-negative bacteria, is known as pathogenic bacteria which induces pneumonia. PAK is recognized by toll-like receptors 4 and 5 and PAK is phagocytosed by co-cultured macrophage at 2 hrs and sustained for 4 hrs. In the PLAG treated cells, phagocytosis of PAK starts at 30 min and maximized at 1 hr and the invading PAK is cleared at 2 hrs. These data indicate that PLAG efficiently enhances the uptakes of invading bacteria and resolves to invade bacteria by a prompt clearance. As shown in FIGS. 5B and 5C, colony formation assay were also performed to confirm the effect of PLAG on bacterial phagocytosis and clearance by counting intracellular PAK in BMDMs and THP-1 cells. This data suggest that PLAG accelerates PAK-induced bacterial engulfment and removal in BMDM.

### Example 6 PLAG Modulates Clearance of Bacteria (PAK) in AC Regimen-Induced Mice Model

Materials and Methods

Animals

Specific pathogen-free male BALB/c mice (6 weeks of age) were purchased from Koatech Corporation (South Korea). The mice were housed in a specific pathogen-free facility under consistent temperature and light cycles. All experimental procedures were approved by the Institutional Animal Care and Use Committee of the Korea Research Institute of Bioscience and Biotechnology performed in compliance with the National Institutes of Health Guidelines for the care and use of laboratory animals and Korean national laws for animal welfare.

AC Regimen-Induced Immunocompromised Mice Model

To establish a chemotherapy-induced immunocompromised mice model, the mice were administered a single intravenous injection of 50 mg/kg cyclophosphamide and 2.5 mg/kg doxorubicin (AC regimen). After 5 days from the injection of AC regimen, blood samples were collected from an intra-orbital vein using EDTA capillary tubes, and the number of circulating neutrophils was measured by complete blood cell count (CBC) analysis using Mindray BC-5300 auto-hematology analyzer (Shenzhen Mindray Bio-medical Electronics, China).

Establishment of PAK-Infected Mice Model and CFU Determination in BALF

Pseudomonas aeruginosa strain K (PAK) was grown overnight in LB broth at 37° C. with agitation and then harvested by centrifugation at 13,000×g for 2 minutes. The pellet was diluted to yield 1×10⁵ colony-forming unit (CFU) per 20 μL of PBS as determined by an optical density 600 nm. The diluted bacteria were administrated to the mice by intranasal injection. Bronchoalveolar lavage fluid (BALF) samples were collected from the PAK-infected mice at different time points after infection in normal mice model and in AC regimen-induced immunocompromised mice model. The harvested BALFs were serially diluted to 1:1000-1:10000 with PBS, and the diluted samples were plated out on LB agar and incubated overnight at 37° C. The number of viable bacteria in BALF was determined by counting the number of colonies formed in the plates.

Results

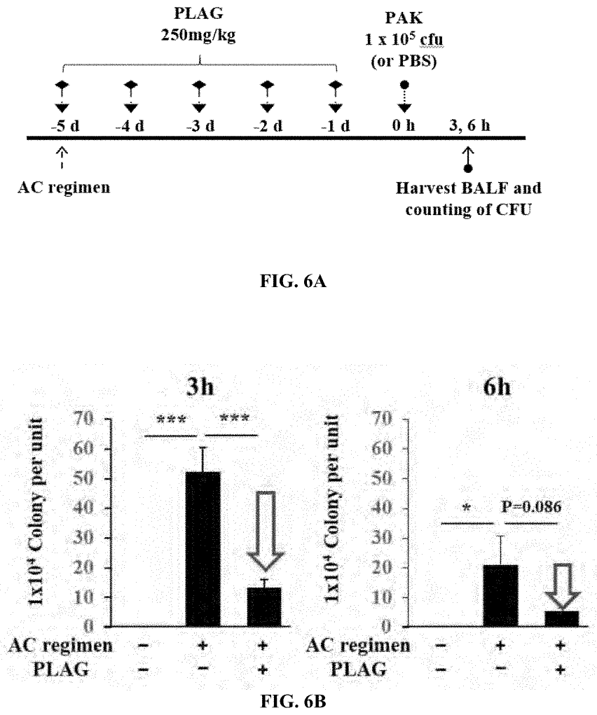

PLAG therapeutic effects on pneumonia were tested in the PAK introduced animal model. The experimental scheme for the evaluation of PLAG's therapeutic efficacy on AC regimen-induced immunocompromised mice model with PAK infection is summarized (FIG. 6A). Immunocompromised mice were prepared by the treatment of chemotherapeutic agents. AC regimen (50 mg/kg of cyclophosphamide and 2.5 mg of doxorubicin) was intravenously administered at a single dose, and PLAG (250 mg/kg) was orally administered once daily for 5 consecutive days. After 5 days, blood samples were collected by retro-orbital bleeding and confirmed the neutropenic condition by using CBC analysis. PAK (1×10⁵ CFU/20 μl) was administered to the AC regimen-treated mice by intranasal inoculation. The BALF samples were harvested at 3 and 6 hours after the infection, and live PAK in BALF after AC regimen and AC regimen/PLAG treatment was determined by counting with colony-forming units (FIG. 6B). These data indicate that PLAG significantly accelerates bacterial clearance in the immunodeficient mice.

### Example 7 PLAG Modulates the Intracellular Trafficking of GPCR

Materials and Methods

HaCaT cells were divided into two groups: 1) IMQ treated group and 2) IMQ/PLAG treated group. For IMQ treated group, cells were stimulated with IMQ (5 μg/ml) for 0, 2.5, 5, 7.5, 10, 15, 30, 60, 120 minutes. For IMQ/PLAG treated group, cells were pre-incubated with PLAG (100 μg/ml) for 1 hour and then stimulated with IMQ (5 μg/ml) for 0, 2.5, 5, 7.5, 10, 15, 30, 60, 120 minutes. (A) To detect ADORA2A on the membrane surface, cells were fixed with 2% paraformaldehyde (Sigma-Aldrich) and were blocked with PBS containing 1% BSA (Gibco, Waltham, Mass., USA). They were incubated with rabbit anti-ADORA2A antibody (Thermo) and Alexa488 conjugated anti-rabbit IgG (Invitrogen) without permeabilization. (B) To detect intracellular ROS, cells were treated with FITC conjugated-CM-H2DCFDA (Invitrogen, Carlsbad, Calif., USA) for 30 min before PLAG treating. For confocal microscopy analysis, cells were washed with PBS and mounted in DAPI-containing fluorescence microscopy mounting medium (Invitrogen). Samples were analyzed with a laser scanning confocal microscope (Carl Zeiss, Oberkochen, Germany).

Results

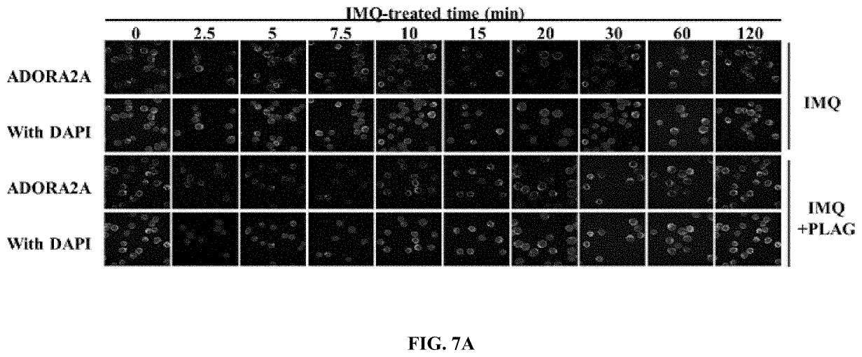

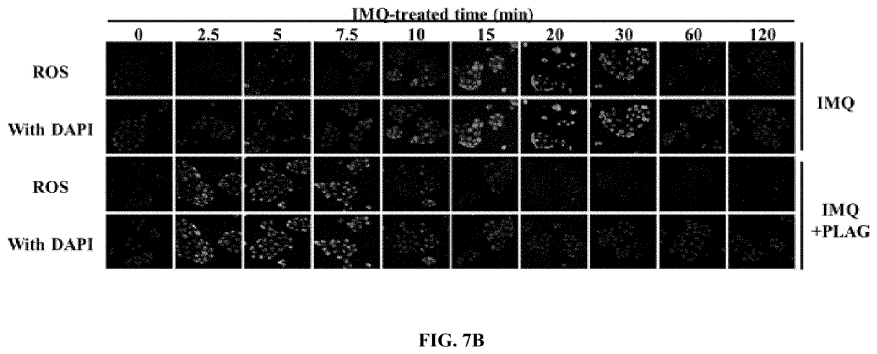

Damage associated molecular patterns (DAMP) molecules are released from cells undergoing apoptotic and necrotic cell death, which has to be removed for homeostasis. As a DAMP molecule, Imiquimod (IMQ) is recognized by adenosine receptor ADORA2A, and the IMQ-bound receptor starts intracellular trafficking at 15 minutes and returns to the membrane at 60 minutes (FIG. 7A). However, for IMQ/PLAG treated cells, GPCR trafficking starts at 2.5 minutes and returns to its membrane at 10 minutes. These data indicate that PLAG significantly accelerates GPCR trafficking. During the GPCR intracellular trafficking, corresponding ROS formation is observed (FIG. 7B). For IMQ treated cells, ROS formation starts at 15 minutes and return to homeostatic levels at 60 minutes. However, for IMQ/PLAG treated cells, ROS formation starts at 2.5 minutes and returns to homeostatic levels at 10 minutes (FIG. 7B). Therefore, these data confirm that PLAG accelerates the intracellular trafficking of GPCR.

### Example 8 PLAG Modulates GPCR Related MAPK Activity

Materials and Methods

Differentiated HaCaT cells were divided into three groups: 1) control (non-treated) group, 2) IMQ treated group, and 3) IMQ/PLAG treated group. For IMQ treated group, cells were stimulated with IMQ (5 μg/ml) for 0, 20, 60 minutes. For IMQ/PLAG treated group, cells were pre-incubated with PLAG (1, 10, 100 μg/ml) for 1 hour and then stimulated with IMQ (5 μg/ml) for an hour. Then, cells were harvested. Cells were ruptured with 1×RIPA lysis buffer (Cell Signaling Technology) containing the protease inhibitor (Roche, Indianapolis, Ind., USA), and phosphatase inhibitor (Thermo Fisher Scientific Inc.) on ice. The cell lysates were then clarified by centrifugation and samples were analyzed on polyacrylamide gels. Each sample was separated by sodium dodecyl sulfate-polyacrylamide gel electrophoresis (SDS-PAGE) on polyacrylamide gels and the proteins were blotted onto a PVDF membrane (Bio-Rad). The membrane was blocked with 5% BSA (Gibco) in PBS containing 0.05% Tween-20 (Merck Millipore, Billerica, Mass., USA). The membrane was incubated with antibodies against phosphor-ERK (Thr202/Tyr204), ERK, phosphor-JNK (Thr183/Tyr185), SAPK/JNK, phosphor-p38MAPK (Thr180/thr182), and p38MAPK, overnight at 4° C. All antibodies were purchased from Cell signaling technology. Target proteins were detected with Immobilon Western Chemiluminescent HRP Substrate (Merck Millipore).

Results

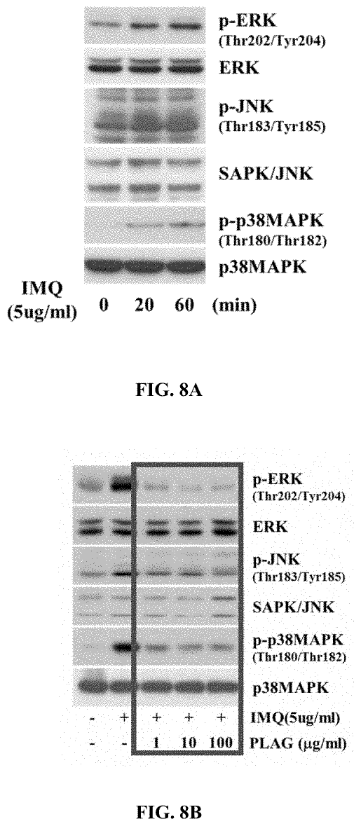

ROS production depends on the time during which GPCR stays in an intracellular endosome. One well known ROS related signals, the MAPK pathway, is activated by ROS. This signal returns to its homeostatic level when DAMP is cleared. As IMQ induces ROS in the cells through GPCR trafficking, phosphorylations of ERK, JNK, and p38MAPK were observed. phosphorylation of ERK, JNK, and p38 MAPK was detected in 20 and 60 minutes for IMQ treatment, as shown from the western blot analysis (FIG. 8A). However, the IMQ-induced phosphorylation of ERK, JBK, and p38 MAPK was attenuated by PLAG in a dose-dependent manner, as shown from the western blot analysis (FIG. 8B). These data indicate that PLAG modulates GPCR related MAPK pathway by modulating the phosphorylation of ERK, JBK, and p38 MAPK.

### Example 9 PLAG Dose-Dependently Modulates the Level of Released Chemokines

Materials and Methods

RAW 264.7 or HaCaT cells were divided into three groups: 1) control (non-treated) group, 2) IMQ treated group, and 3) IMQ/PLAG treated group. For IMQ treated group, cells were treated with 1, 10, 100 μg/ml of DMSO (as solvent control) for 1 hour and treated with 5 μg/mL of IMQ for 12 hours. For IMQ/PLAG treated group, cells were treated with 1, 10, 100 μg/ml of PLAG for 1 hour and treated with 5 μg/mL of IMQ for 12 hours. MIP-2 (A), IL-6 (B) and CXCL8 (C) in the culture supernatants were analyzed with the cognate antibody using ELISA kit. Modulation of CXCL8 expression in the IMQ treated HaCaT cells by MAPK inhibitors, SCH 772984 (ERK inhibitor, (D)), SP600125 (JNK inhibitor, (E)) and SB203580 (p38 inhibitor, (F)) was evaluated using ELISA kit (BD bioscience, New Jersey, USA) according to the manufacturer's instructions. The cytokine levels were estimated by interpolation from a standard curve using an ELISA reader (Molecular Devices, Sunnyvale, USA) at 450 nm.

Results

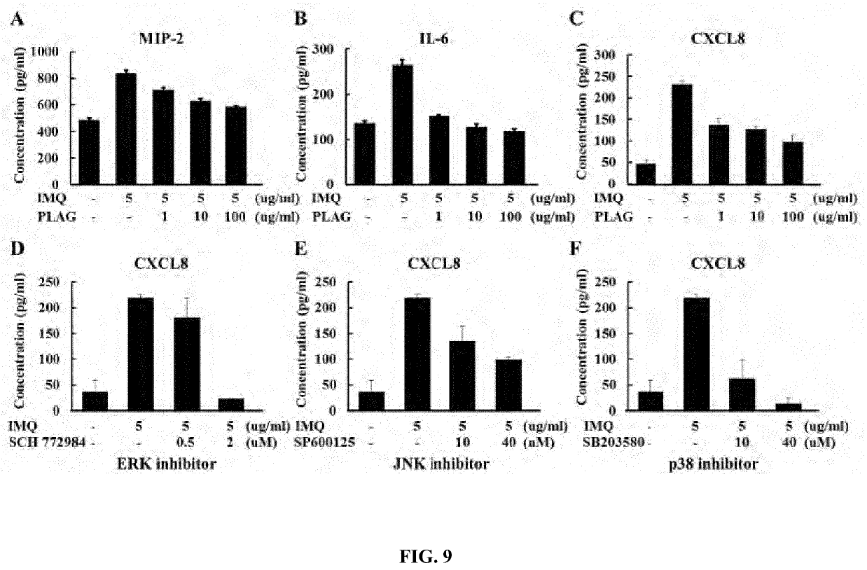

As a consequence of ROS signaling, chemokines, MIP-2, IL-6, and CXCL8 were significantly induced by IMQ (FIG. 9, upper row). In the IMQ/PLAG treated cells, chemokines and cytokines (MIP-2, IL-6, CXCL8) induced by IMQ were gradually decreased in a dose-dependent manner (FIG. 9, upper row). It was verified that the expression of CXCL8 is dependent on the MAPK signaling pathway by using ERK inhibitor (SCH772984), JNK inhibitor (SP600125) and p38 (SB203580) (FIG. 9, lower row). These data indicate that PLAG dose-dependently modulates the level of chemokines by modulating MAPK signaling pathway.

### Example 10 PLAG Modulates IMQ-Induced Psoriasis

Materials and Methods

Mice

BALM mice were obtained from Koatech Co. (Pyongtaek, Republic of Korea) and were 8-10 weeks of age and 21-23 grams at the time of the experiments. These mice were maintained on a regular 12 hours light-12 hours dark cycle at 24° C. with 40-60% humidity and preserved under specific pathogen-free conditions. All animal experimental procedures were performed in accordance with the Guide and Use of Laboratory Animals (Institute of Laboratory Animal Resources).

Psoriasis Experimental Model and Scoring

Mice were daily treated with 40 mg of Aldara cream (3M Health Care Limited, England), which contains 5% imiquimod (IMQ), on the shaved back, and one ear for 5 days. Vaseline (Unilever, United Kingdom) was used as a control vehicle cream. PLAG (Enzychem Lifesciences Co., Daejeon, Republic of Korea) were diluted in phosphate-buffered saline (PBS, Wellgene, Daegu, Republic of Korea) and administered orally with 250 mg/kg body weight for 5 days using feeding needle catheter every day. Control (non-treated) and IMQ-treated groups were administrated orally with the same PBS daily. Psoriasis was scored by a blinded investigator using published criteria based on the following parameters: erythema, scaling, and thickening.

Hematoxylin and Eosin Staining, Immunohistochemistry (IHC)

Back skin specimens were fixed in 10% buffered formalin for 24 hr, embedded in paraffin, and sectioned at 4 μm. The tissue sections were stained with hematoxylin and eosin (H&E). For IHC analyses, back skin serial sections were cut and mounted on charged glass slides (Superfrost Plus; Fisher Scientific, Rochester, N.Y., USA). The sections were deparaffinized and then treated with 3% hydrogen peroxide in methanol to quench the endogenous peroxidase activity. Samples were then incubated with 1% BSA to block non-specific binding. The sections were incubated with the primary anti-mouse IL-17 (abcam) antibody (1:100) or rat anti-mouse neutrophil (NIMP-R14, Thermo Fisher Scientific Inc.) antibody (1:100) at 4° C. overnight. After washing with TBS, the slides were incubated with 1:250 dilution of secondary antibody at room temperature for 15 min. The tissue sections were immersed in 3-amino-9-ethylcarbazole (AEC, Dako, Denmark) as a substrate, and then samples were counterstained with 10% Mayer's hematoxylin, dehydrated, and mounted with a crystal mount. An irrelevant goat IgG of the same isotype and antibody dilution solution served as a negative control. Images were observed under light microscopy (Olympus).

Results

Psoriasis is regarded as a common inflammatory disease triggered by damage-associated molecular patterns (DAMPs) showing phenotypes like as proliferation of keratinocytes and infiltration of excessive neutrophils into dermis and epidermis. Imiquimod (IMQ), a DAMP molecule, is commonly used to develop psoriasis-like skin inflammation in the mice. As the main pathogenesis of psoriasis, IMQ stimulates epithelial cells and tissue-resident macrophages and results in the secretion of chemo-attractants which initiate neutrophil recruitment into a lesion. Daily application of IMQ on mouse back skin induced inflamed scaly skin lesions resembling plaque-type psoriasis. These lesions showed increased epidermal proliferation, abnormal differentiation, and epidermal accumulation of neutrophils in the micro abscess. Synthetic diacylglycerol derivatives, 1-palmitoyl-2-linoleoyl-3-acetyl-rac-glycerol (PLAG) was studied for evaluation of its therapeutic efficacy on IMQ-induced psoriasis-like skin inflammation.

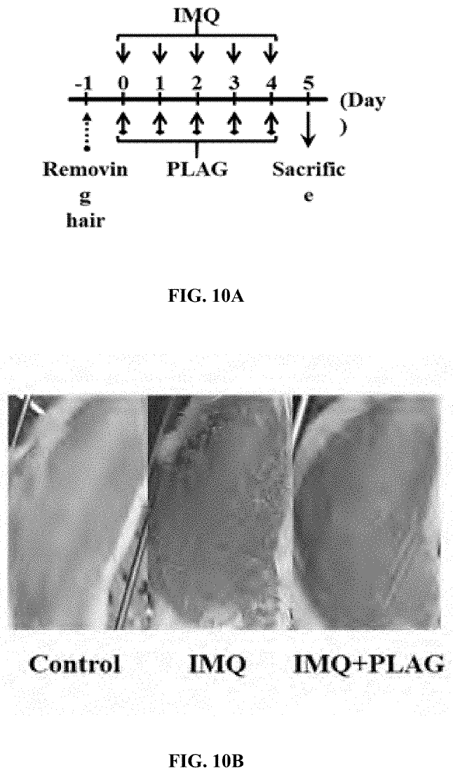

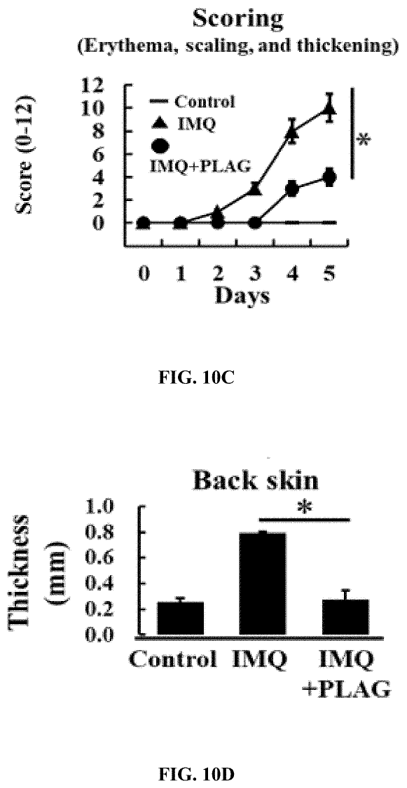

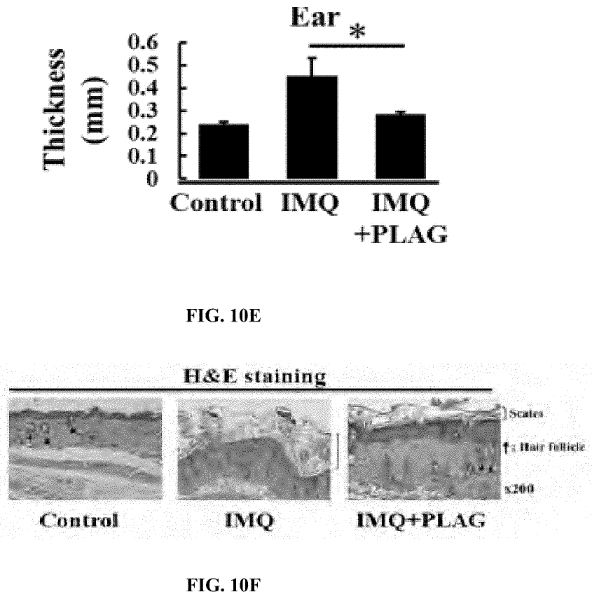

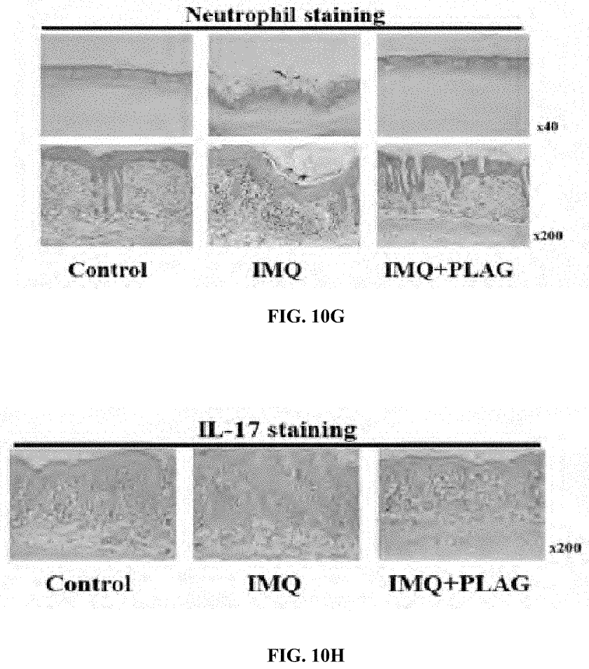

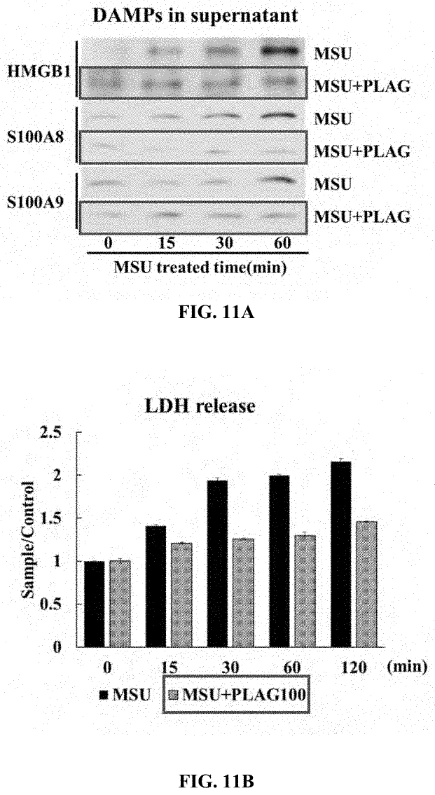

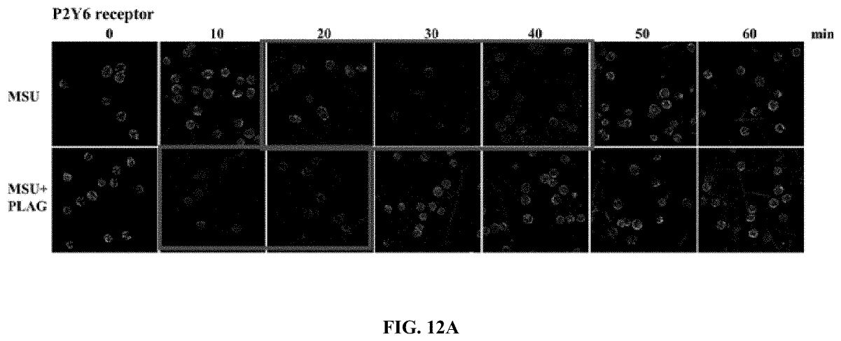

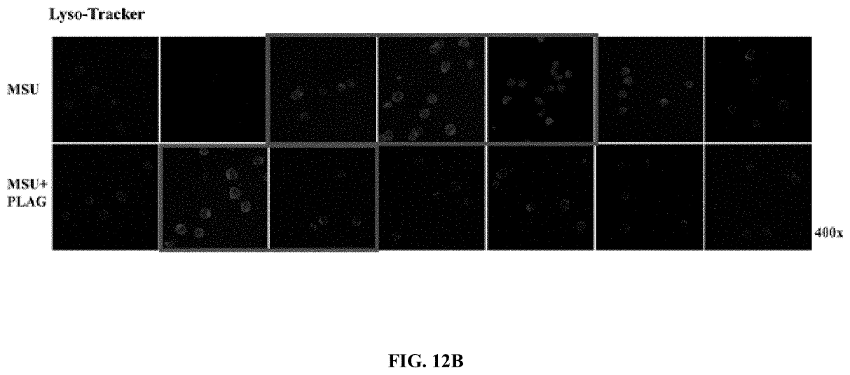

First, mice were divided into three groups: 1) control group, 2) IMQ cream-treated group, and 3) IMQ cream/PLAG co-treated group. Mice were treated with IMQ cream on the shaved back and one ear every day for 5 days. PLAG were administered 250 mg/kg/day orally. On Day 5, mice were sacrificed and the isolated tissues were analyzed (FIG. 10A). On Day 5, mice were sacrificed and the isolated back skin tissues of control, IMQ treated and IMQ/PLAG co-treated mice were analyzed (FIG. 10B). IMQ is used as an agent for psoriasis induction in the animal model. The scoring of control, IMQ treated, and IMQ/PLAG co-treated mice calculated by a blinded investigator using published criteria based on the following parameters: erythema, scaling, and thickening (FIG. 10C). IMQ-treated back skin and one ear were isolated and skin thickness was measured (FIGS. 10D and 10E). The back skins were isolated and stained with H&E (FIG. 10F). The back skins were stained with anti-neutrophil or anti-IL-17 antibodies (FIGS. 10G and 10H). PLAG turned out to help the back skins to maintain their structural integrity. These data indicate that DAMP-induced symptoms such as IMQ-induced psoriasis can be effectively resolved by PLAG.

### Example 11 PLAG Modulates the Release of MSU-Induced DAMP and LDH Molecules

Materials and Methods

THP-1 cells were divided into two groups: 1) MSU treated group and 2) MSU/PLAG treated group. For MSU treated group, THP-1 cells were stimulated with monosodium urate (MSU) crystal (400 μg/ml) for 0, 15, 30, 60 minutes. For MSU/PLAG treated group, THP-1 cells were pre-incubated with PLAG (100 μg/ml) for 1 hour and then stimulated with monosodium urate (MSU) crystal (400 μg/ml) for 0, 15, 30, 60 minutes. Then, cells were centrifuged, and the supernatant was harvested. Add 50 μL 5×SDS sample buffer to 200 μL supernatant of THP-1 cells. Proteins from each sample were separated by sodium dodecyl sulfate (SDS)-polyacrylamide gel electrophoresis on 8% polyacrylamide gels and the proteins were blotted onto a PVDF membrane (Millipore Corporation, Germany). The membrane was blocked with 5% non-fat dried milk (BD bioscience) in PBS containing 0.05% Tween-20 (Calbiochem) for 1 hour. The membrane was incubated with anti-high mobility group box 1 (HMGB1) (abcam), anti-S100A9 (abcam) at 4° C. overnight. After washing with PBS containing 0.05% Tween-20, the membrane was stained with goat anti-rabbit IgG peroxidase (ENZO). Target proteins were detected with Immobilon Western Chemiluminescent HRP Substrate (Millipore Corporation). Lactate dehydrogenase (LDH) of Supernatant was measured using the LDH assay kit.

Results

MSU (monosodium urate) crystal-induced release of DAMP molecules such as HMGB1, S100A8, and S100A9 and cytosolic enzyme such as LDH to the supernatant. PLAG modulated the release of MSU crystal-induced HMGB1, S100A8, and S100A9, as shown from western blot analysis (FIG. 11A) and cytosolic enzyme LDH release to the supernatant (FIG. 11B). These data indicate that PLAG can modulate the release of MSU-induced DAMP molecules and cytosolic enzymes.

### Example 12 PLAG Modulates MSU-Induced P2Y6 Receptor Trafficking

Materials and Methods

THP-1 cells were divided into two groups: 1) MSU treated group and 2) MSU/PLAG treated group. For MSU treated group, THP-1 cells were stimulated with monosodium urate (MSU) crystal (400 μg/ml) for 0, 10, 20, 30, 40, 50, 60 minutes. For MSU/PLAG treated group, THP-1 cells were pre-incubated with PLAG (100 μg/ml) for an hour and then stimulated with monosodium urate (MSU) crystal (400 μg/ml) for 0, 10, 20, 30, 40, 50, 60 minutes. Then, cells were harvested. (A) To detect for the P2Y6 receptor on the membrane surface, cells were fixed with 4% paraformaldehyde (Sigma-Aldrich) and incubated with PBS containing 1% BSA for blocking. They were labeled with the rabbit anti-P2Y6 receptor antibody (1:200, APR-011, Alomone Labs, Jerusalem, Israel) for 1 h. The detection Ab was used with Alexa Flour 488 goat anti-rabbit IgG (Invitrogen). (B) To detect lysosomal activity, THP-1 cells were stained with Texas red conjugated-LYSO-ID® Lysosomal Detection Kit (Enzo Life Sciences, Inc.). Finally, cells were washed with 1% FBS/PBS and mounted with DAPI-containing fluorescence microscopy mounting medium (Invitrogen). Confocal samples were analyzed with a laser scanning confocal microscope (Carl Zeiss, Oberkochen, Germany). For flow cytometric analysis, cells were washed and analyzed with a FACSVerse flow cytometer (BD Biosciences). FlowJo software (Tree Star, OR, USA) was used data processing.

Results

Confocal microscopy of P2Y6 receptors after MSU treatment shows that P2Y6 recognizing MSU crystal initiates endocytosis at about 20 minutes and returned to the surface at about 50 minutes (FIG. 12A, upper row). During P2Y6 trafficking, lysosomal activity determined by Lyso-Tracker was observed at about 20 minutes and returned to the surface at about 50 minutes (FIG. 12B, upper row). In the MSU/PLAG treated cells, endocytosis initiates at about 10 minutes and returned to the surface at about 30 minutes (FIG. 12A, lower row). The lysosomal activity was also detected early at about 10 minutes and returned to the surface at about 30 minutes (FIG. 12B, lower row). These results indicate that PLAG modulates MSU-induced P2Y6 receptor trafficking.

### Example 13 PLAG Modulates Phosphorylation of RIPK1, RIPK3, and MLKL

Materials and Methods

(A) THP-1 cells were pre-incubated with PLAG (100 μg/mL) for an hour and then stimulated with MSU crystal (400 μg/mL). After 0, 7, 15, 30, 60 minutes, cells were harvested. (B) THP-1 cells were pre-incubated with PLAG (1, 10, 100 μg/mL) for 1 hour and then stimulated with MSU crystal (400 μg/mL). After 1 h, cells were harvested. THP-1 cells were lysis with 1×RIPA lysis buffer containing the protease inhibitor (Roche, Basel, Switzerland) and phosphatase inhibitor (Thermo Scientific, MA, USA) on ice for 30 min. The cell lysates were clarified by centrifugation (13,000 rpm, 4° C., 30 min), and the protein quantity from each sample was examined by Bradford assay (Bio-Rad). Proteins from each sample were separated by sodium dodecyl sulfate-polyacrylamide gel electrophoresis on 12% polyacrylamide gels and the proteins were blotted onto a PVDF membrane (Millipore Corporation, Germany). The membrane was blocked with 5% non-fat dried milk (BD bioscience) in PBS containing 0.05% Tween-20 (Calbiochem) for 1 hour. The membrane was incubated with anti-phospho-RIPK3 (abcam), RIPK3 (novusbio), p-MLKL (abcam), MLKL (Sigma), p-RIP (Cell Signaling Technology), RIPK1 (R&D System) and β-actin (Cell Signaling Technology) at 4° C. overnight. After washing with PBS containing 0.05% Tween-20, the membrane was stained with goat anti-rabbit IgG peroxidase (ENZO). Target proteins were detected with Immobilon Western Chemiluminescent HRP Substrate (Millipore Corporation).

Results

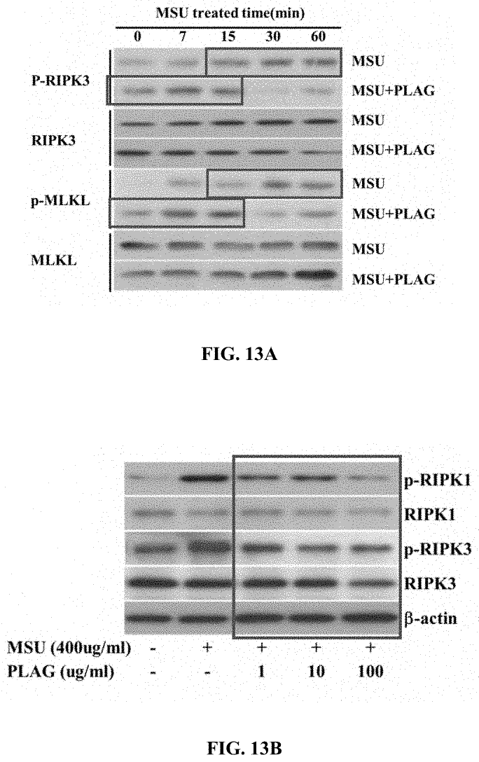

MSU crystal treatment phosphorylated RIPK3 and MLKL to P-RIPK3 and P-MLKL, respectively, as shown by the western blot (FIG. 13A, MSU). PLAG accelerated the MSU-induced phosphorylation of RIPK3 and MLKL, thereby promoting earlier initiation and shorter duration thereof (FIG. 13A, MSU+PLAG). PLAG modulated the MSU-induced phosphorylation of RIPK1 and RIPK3 in a dose-dependent manner, as shown by the western blot (FIG. 13B). These data indicate that PLAG dose-dependently modulates the phosphorylation of RIPK1, RIPK3, and MLKL by accelerating the process.

### Example 14 PLAG Modulates the NETosis of PAK-Introduced Bone Marrow-Derived Cells

Materials and Methods

2 hours after PAK infection, bone marrow-derived cells were harvested and detected extracellular DNA-elastase complex by using ELISA and visualized by using confocal microscopy (×400) or scanning electron microscope (SEM) (×8000). HL-60 cells were washed with PBS and mounted in DAPI-containing fluorescence microscopy mounting medium (Invitrogen). Samples were analyzed with a laser scanning confocal microscope (Carl Zeiss, Oberkochen, Germany).

Results

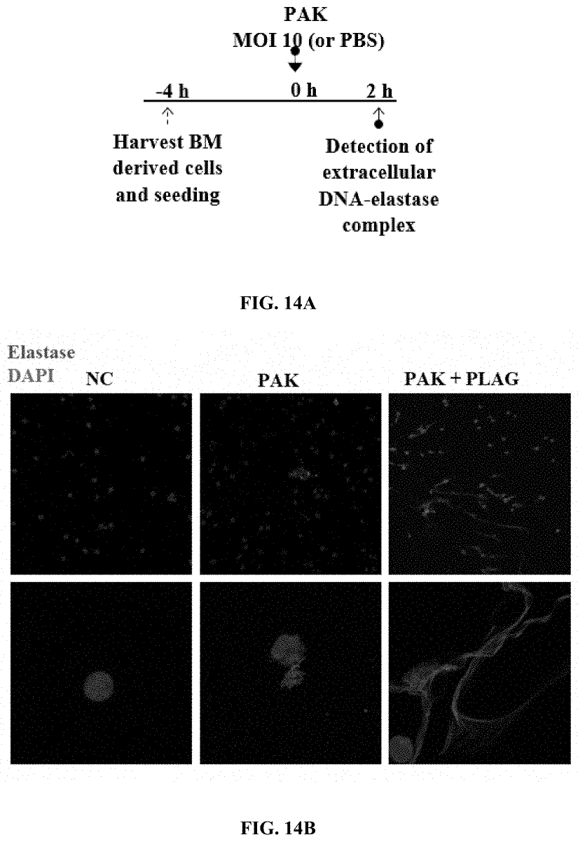

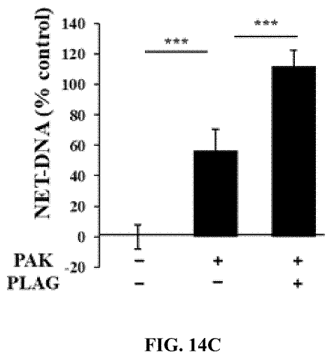

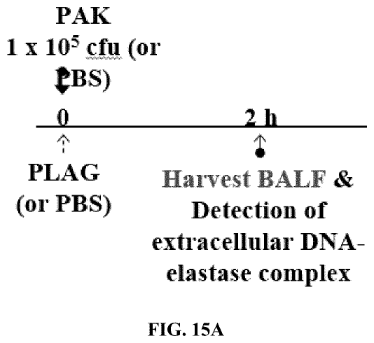

To show how PLAG affects on netosis, PAK-induced bone marrowderived cells were studied. The experimental scheme is illustrated as a diagram. (FIG. 14A). Net formation of neutrophil is accelerated in the PAK/PLAG treated bone marrow-derived cells compared to PAK-introduced bone marrow-derived cells as shown by the confocal microscopy (FIG. 14B). ELISA results show PLAG's effect on the formation of extracellular DNA-elastase complex (FIG. 14C). These data indicate that PLAG promotes the NETosis of PAK-introduced bone marrow-derived cells.

### Example 15 PLAG Modulates the NETosis of PAK Introduced BALF Derived Cells

Materials and Methods

2 hours after PAK infection, BALF derived cells were harvested and detected extracellular DNA-elastase complex by using ELISA and visualized by using confocal microscopy (×400) or scanning electron microscope (SEM) (×8000). HL-60 cells were washed with PBS and mounted in DAPI-containing fluorescence microscopy mounting medium (Invitrogen). Samples were analyzed with a laser scanning confocal microscope (Carl Zeiss, Oberkochen, Germany).

Results

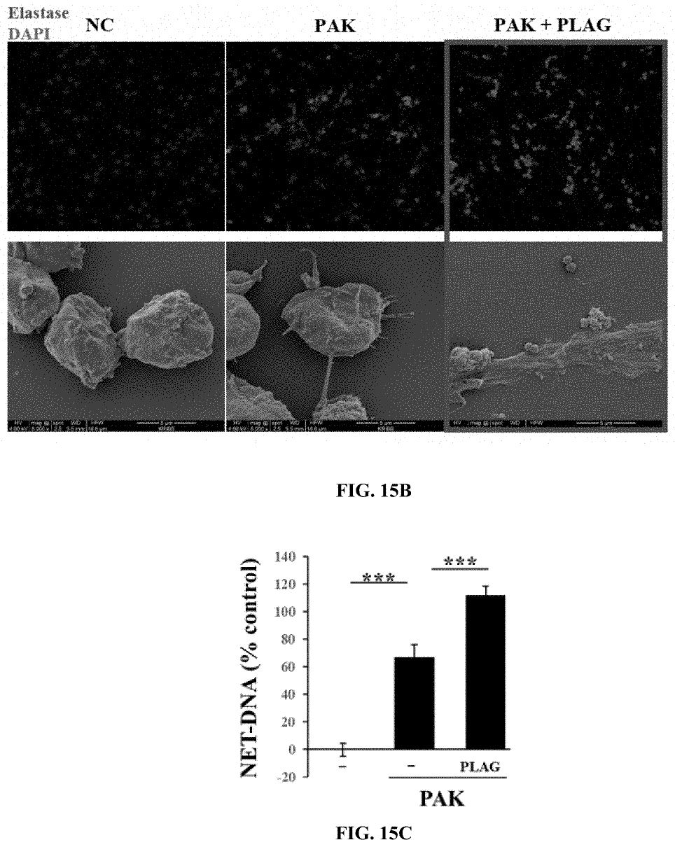

To show how PLAG affects on NETosis, PAK-induced BALF derived cells were studied. The experimental scheme is summarized as a diagram (FIG. 15A). Net formation of neutrophil is accelerated in the PAK/PLAG treated BALF derived cells compared to PAK-introduced BALF derived cells (FIG. 15B). ELISA results show PLAG's effect on the formation of extracellular DNA-elastase complex (FIG. 15C). These data indicate that PLAG promotes the NETosis of PAK-introduced BALF derived cells.

### Example 16 PLAG Modulates Intracellular Calcium Mobilization in Differentiated Human Leukemia Line (dHL-60) Cells

Materials and Methods

Fluo-4 Calcium Assay

Human leukemia line (HL-60) cells were differentiated (dHL-60) to neutrophil-like cells in the culture medium with the addition of 1.3% DMSO (Sigma) for 5 days in a humidified atmosphere at 37° C. without changing the medium. dHL-60 cells (2×10⁵ cells/mL) were loaded with 5 μM fluo-4 AM for 45 minutes and washed three times with warmed modified (37° C.) HBSS buffer (137.93 mM NaCl, 5.33 mM KCl, 2 mM CaCl₂), 1 mM MgSO₄, 2.38 mM HEPES, 5.5 mM glucose, pH to 7.4). The cells were seeded on black-walled 96-well plates and then treated with vehicle (0.1% DMSO), ionomycin (5 μM; positive control) or PLAG (100 μg/mL) just before measurement. Baseline fluorescence was measured before treatment, and fluorescence was read every 20 s for 700 s using an excitation wavelength of 494 nm, an emission wavelength of 516 nm in a FlexStation 3 microplate reader (Molecular Devices). Fluorescence values were reported as F/Fo according to the calculation: [ΔF=(494 nm)f/(516 nm)f−(494 nm)₀/(516 nm)₀]. For studies in 0 mM external calcium, the cells were loaded as mentioned earlier. After being loaded, the cells were washed four times with calcium-free HBSS. The remainder of the study was carried out in calcium-free HBSS.

Western Blot Analysis

dHL-60 cells were lysed on ice for 30 minutes in RIPA buffer composed of 50 mM Tris-HCl (pH 7.4), 150 mM NaCl, 2 mM EDTA, 1% NP-40, 0.5% sodium deoxycholate, 0.1% SDS, 1 mM phenylmethylsulfonyl fluoride (PMSF), 10 mM sodium fluoride, 2 mM sodium pyrophosphate, 10 mM β-glycerophosphate, 10 mM sodium orthovanadate. The lysates were centrifuged at 13,000 rpm for 20 minutes at 4° C. and protein concentrations were determined using the Bradford assay (Bio-Rad Laboratories). Denatured samples were mixed with a 5×SDS-PAGE loading buffer and heated to 100° C. for 15 min. The samples were separated on the 10% of SDS-PAGE gel and transferred to polyvinylidene difluoride (PVDF) membranes (Merck Millipore Corporation, MA, USA). Membranes were blocked with 5% non-fat milk in PBS (10 mM Tris-HCl, pH7.5, 150 mM NaCl) for 1 h and probed with primary antibodies against histone H3 (citrulline R2+R8+R17) from abcam (USA) and β-actin from cell signaling (USA) for overnight at 4° C. The blots were washed and incubated with appropriate secondary antibodies and visualized using Pierce™ ECL Western Blotting Substrate (Thermo Scientific).

Results

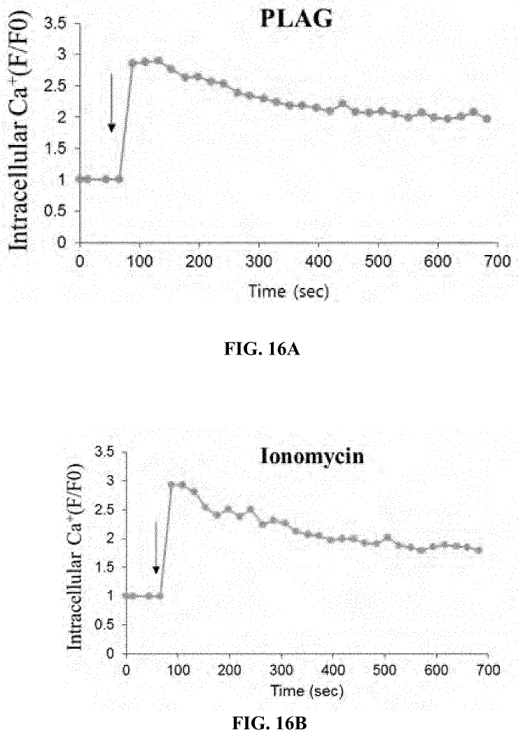

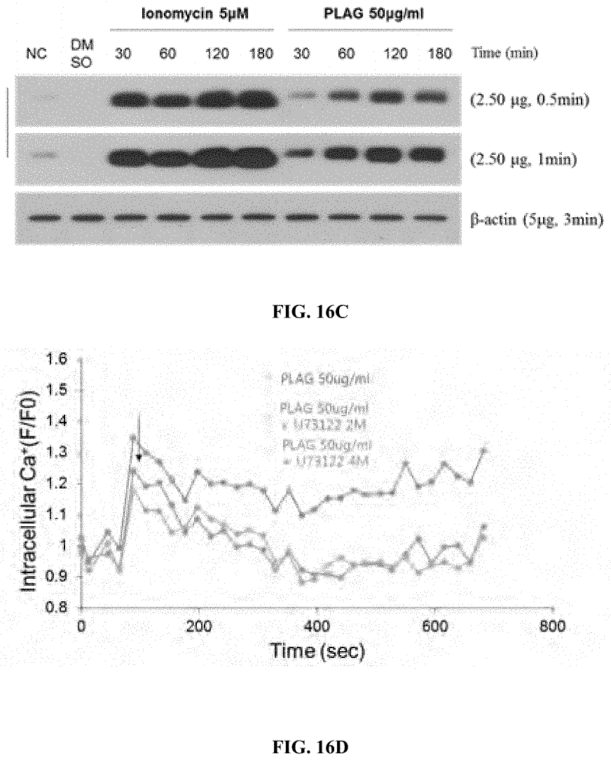

Since PLAG increased PAD4-dependent neutrophil extracellular traps (NETs) formation of dHL-60 cells in PAK-infected condition, it was next investigated whether PLAG increases intracellular calcium levels using a calcium indicator, fluo-4 AM, and fluorescence microplate reader. PLAG treatment increased cytosolic calcium of dHL-60 cells in the same manner as ionomycin treatment (FIGS. 16A and 16B). The nuclear translocation of PAD4 is essential for histone citrullination during calcium-dependent NETosis. Western blot analysis was performed to investigate whether PLAG increases the citrullination of histone H3 in dHL-60 cells. Like ionomycin treatment, PLAG induced histone H3 citrullination in a time-dependent manner, as shown by the western blot (FIG. 16C). Phospholipase C (PLC) is a major signaling molecule responsible for intracellular calcium mobilization. It was next investigated whether the intracellular calcium increase by PLAG is dependent on PLC signaling by using PLC inhibitor, U73122. U73122 inhibited PLAG-induced intracellular calcium increase in a dose-dependent manner (FIG. 16D). These results indicate that PLAG increases intracellular calcium levels and histone citrullination via the activation of PLC signaling in neutrophils.

### Example 17 PLAG Modulates IMQ-Induced Intracellular Calcium Mobilization in Differentiated Human Leukemia Line (dHL-60) Cells

Materials and Methods

Fluo-4 Calcium Assay

HL-60 cells were differentiated (dHL-60) to neutrophil-like cells in the culture medium with the addition of 1.3% DMSO (Sigma) for 5 days in a humidified atmosphere at 37° C. without changing the medium. dHL-60 cells (2×10\hat{ }5 cells/mL) were loaded with 5 μM fluo-4 AM for 45 minutes and washed three times with warmed modified (37° C.) HBSS buffer (137.93 mM NaCl, 5.33 mM KCl, 2 mM CaCl2, 1 mM MgSO4, 2.38 mM HEPES, 5.5 mM glucose, pH to 7.4). The cells were seeded on black-walled 96-well plates and then treated with vehicle (0.1% DMSO), imiquimod (10 μg/mL) or PLAG (100 μg/mL) just before measurement. Baseline fluorescence was measured before treatment, and fluorescence was read every 20 seconds for 700 seconds using an excitation wavelength of 494 nm, an emission wavelength of 516 nm in a FlexStation 3 microplate reader (Molecular Devices). Fluorescence values were reported as F/Fo according to the calculation: [ΔF=(494 nm)f/(516 nm)f−(494 nm)0/(516 nm)0]. For studies in 0 mM external calcium, the cells were loaded as mentioned earlier. After being loaded, the cells were washed four times with calcium-free HBSS. The remainder of the study was carried out in calcium-free HBSS.

Results

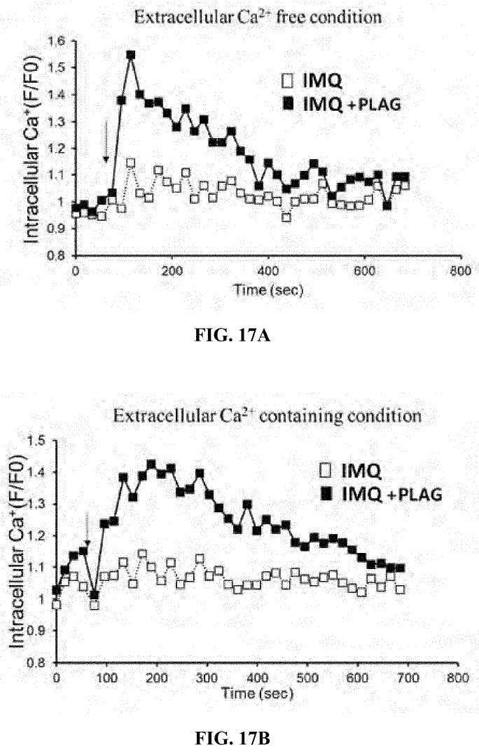

Psoriasis is a persistent inflammatory skin disease characterized by chronic IL-17 and IFNα production. Imiquimod is a TLR7 and adenosine receptor agonist commonly used as an inducer of psoriasis in the animal model. Various studies reported that neutrophils were recruited to psoriasis lesions, particularly in the epidermis, and that neutrophils in psoriasis sera were more prone to form NETs. In this study, it was investigated whether PLAG and imiquimod increase intracellular calcium levels in dHL-60 cells using a calcium indicator, fluo-4 AM, and fluorescence microplate reader. Co-treatment with PLAG significantly increased intracellular calcium levels in dHL-60 cells as compared to the single treatment of imiquimod in both extracellular calcium-free and containing condition (FIGS. 17A and 17B). This result suggests that PLAG may increase the formation of NETs in neutrophils in the imiquimod-induced psoriasis model.

### Example 18 PLAG Dose-Dependently Modulates the NETosis of IMQ Induced Differentiated Human Leukemia (dHL-60) Cells

Materials and Methods

Fluo-4 Calcium Assay

HL-60 cells were differentiated (dHL-60) to neutrophil-like cells in the culture medium with the addition of 1.3% DMSO (Sigma) for 5 days in a humidified atmosphere at 37° C. without changing the medium. dHL-60 cells (2×10\hat{ }5 cells/mL) were loaded with 5 μM fluo-4 AM for 45 minutes and washed three times with warmed modified (37° C.) HBSS buffer (137.93 mM NaCl, 5.33 mM KCl, 2 mM CaCl2, 1 mM MgSO4, 2.38 mM HEPES, 5.5 mM glucose, pH to 7.4). The cells were seeded on black-walled 96-well plates and then treated with vehicle (0.1% DMSO), imiquimod (10 μg/mL) or PLAG (10 or 100 μg/mL) just before measurement. Baseline fluorescence was measured before treatment, and fluorescence was read every 20 s for 700 s using an excitation wavelength of 494 nm, an emission wavelength of 516 nm in a FlexStation 3 microplate reader (Molecular Devices). Fluorescence values were reported as F/Fo according to the calculation: [ΔF=(494 nm)f/(516 nm)f−(494 nm)0/(516 nm)0]. For studies in 0 mM external calcium, the cells were loaded as mentioned earlier. After being loaded, the cells were washed four times with calcium-free HBSS. The remainder of the study was carried out in calcium-free HBSS.

Confocal Microscopy

HL-60 cells were harvested and detected extracellular DNA-elastase complex by using ELISA and visualized by using confocal microscopy (×400) or scanning electron microscope (SEM) (×8000). HL-60 cells were washed with PBS and mounted in DAPI-containing fluorescence microscopy mounting medium (Invitrogen). Samples were analyzed with a laser scanning confocal microscope (Carl Zeiss, Oberkochen, Germany).

Results

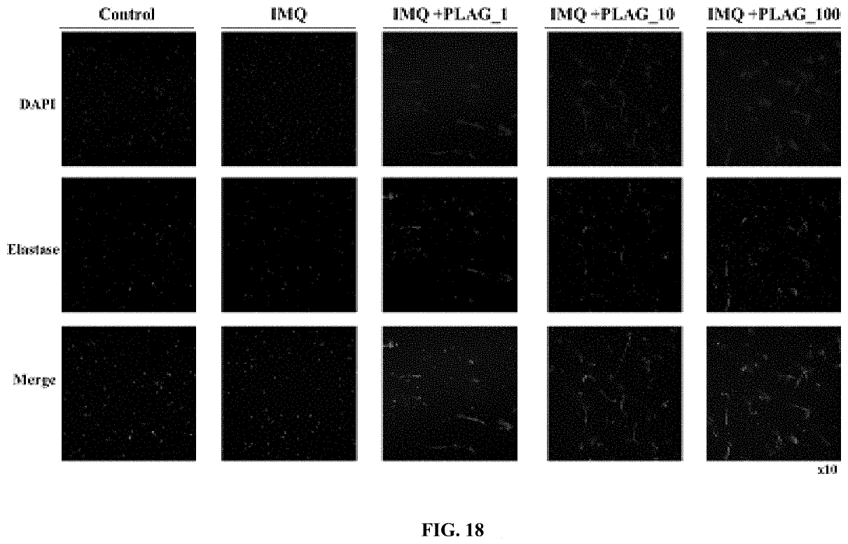

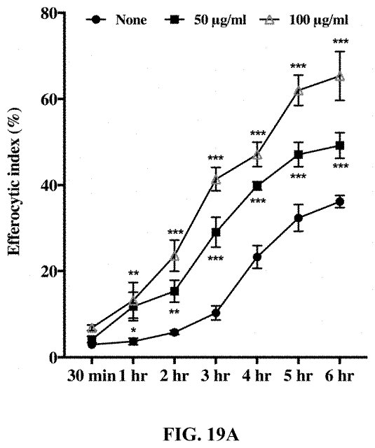

The process of NETosis in neutrophils after IMQ treatment and IMQ/PLAG treatment was studied. PLAG promotes IMQ-induced NETosis in a dose-dependent manner, as observed in the confocal microscopy of extracellular DNA-elastase complex formed by NETosis (FIG. 18). The result indicates that PLAG increases intracellular calcium concentration and successively promotes neutrophil NET formation under the IMQ treated condition.

### Example 19 PLAG Modulates the Clearance of Apoptotic Neutrophils

Materials and Methods

Cell Culture

THP-1 and HL60 cells were obtained from the American Type Culture Collection (ATCC, Rockville, Md., USA). THP-1 cells were grown in RPMI1640 medium (WELGENE, Seoul, Korea) containing 10% fetal bovine serum (HyClone, Waltham, Mass., USA), 1% antibiotics (100 mg/l streptomycin, 100 U/ml penicillin), and 0.4% 2-Mercaptoethanol (Sigma Aldrich, St. Louis, Mo., USA). HL60 cells were grown in RPMI1640 medium containing 20% fetal bovine serum and 1% antibiotics (100 mg/l streptomycin, 100 U/ml penicillin). Cells were grown at 37° C. in a 5% CO2 atmosphere. To differentiate THP-1 cells into macrophage-like cells, cells were grown in medium with 1% Phorbol 12-myristate 13-acetate (PMA) (Sigma Aldrich) for 72 h. To differentiate THP-1 cells into neutrophil-like cells, cells were grown in medium with 10% DMSO (Sigma Aldrich) for 5 days

Determination of Efferocytosis Index and Apoptotic Cell Clearance

Differentiation HL60 was stained with 10 mM CellTracker Red CMTPX (Molecular probes, Eugene, OG, USA) for 30 minutes in PBS and lead to apoptosis by PMA treatment for 24 hours. Differentiation THP-1 were stained with 10 mM CellTracker Green CMFDA (Molecular probes, Eugene, OG, USA) for 30 minutes in PBS. Apoptotic neutrophils were co-culture with macrophage and harvest. Cells were washed twice using PBS and resuspended PBS contained 0.2% BSA. Efferocytotic index was analyzed by FACS (BD bioscience, Franklin Lakes, N.J., USA). In the control group, clearance of apoptotic neutrophils by macrophage phagocytosis was observed by confocal microscopy within 2 hours, whereas in the PLAG-treated (100 μg/mL) group, this was seen within 30 minutes.

Live-Cell Image

Differentiation HL60 was stained with 10 mM CellTracker Red CMTPX (Molecular probes, Eugene, OG, USA) for 30 minutes in PBS and lead to apoptosis by PMA treatment. Differentiation THP-1 were stained with 10 mM CellTracker Green CMFDA (Molecular probes, Eugene, OG, USA) for 30 minutes in PBS. The co-culture plate was put on the stage of LSM800 (Carl Zeiss, Thornwood, NY, USA) for 120 min. Fluorescence overlay videos were recorded using ZEN program (Carl Zeiss, Thornwood, NY, USA)

Results

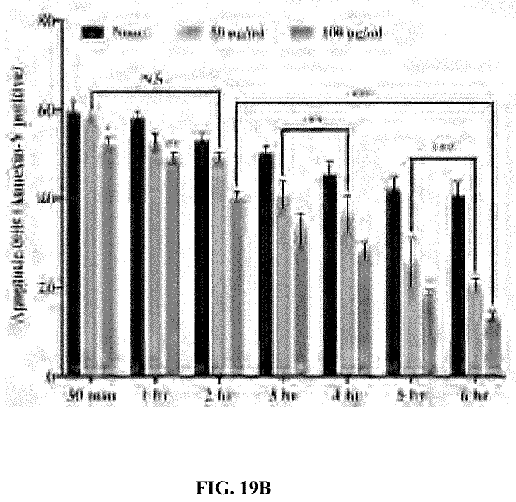

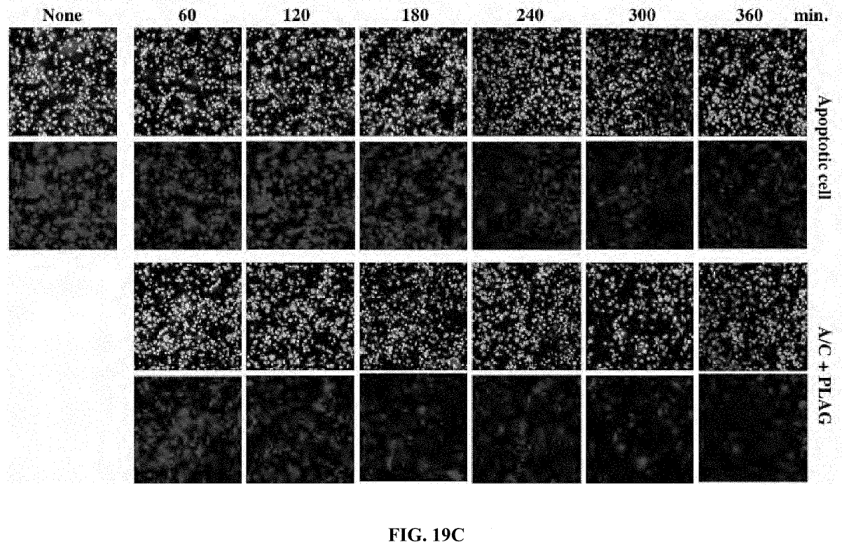

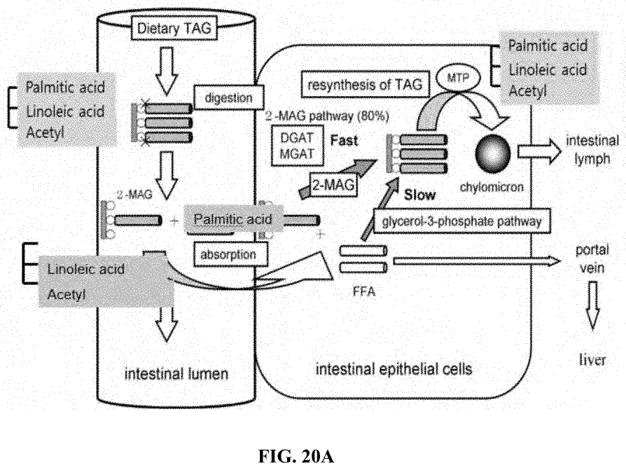

PLAG promotes efferocytosis of apoptotic neutrophils dose-dependently, as shown by the efferocytotic index over time after PLAG treatment (FIG. 19A). PLAG effectively eliminates dead neutrophils in a dose-dependent fashion (FIG. 19B). PLAG having effects on the clearance of apoptotic neutrophils through the enhanced efferocytosis activity using a confocal microscope. Red cells are apoptotic neutrophils, and for PLAG-treated groups, efferocytosis is accelerated, and thus there are fewer dead neutrophils (FIG. 19C). These data indicate that PLAG promotes the clearance of apoptotic neutrophils.

### Example 20 Schematics of PLAG Delivery from the Intestinal Lumen to Lymphatic Vessels

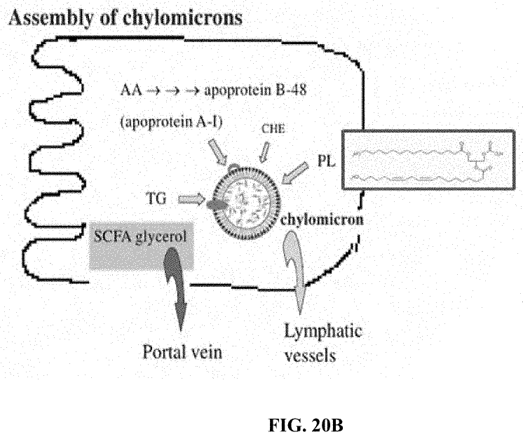

Lipids in diets are absorbed through the intestinal epithelial cell as fatty acid and monoacyl-glyceride (2MAG). PLAG is first absorbed through enterocytes from the intestinal lumen, as shown by the comprehensive schematics of PLAG delivery from the intestinal lumen to lymphatic vessels (FIG. 20A). Generally, dietary TAG is digested in the intestinal lumen and 2-monoacyl glyceride (2MAG) and fatty acid are absorbed into intestinal epithelial cells. There, TAG is reconstituted with aid of MGAT and DGAT enzymes and assembled as chylomicrons (FIG. 20B). Chylomicron is a kind of vesicle that contains TG and cholesterol and lipoprotein. Chylomicron is trafficked via intestinal lymphatic vessel (lacteal duct).

### Example 21 PLAG Uptake in the Cisterna Chyli

Materials and Methods

Balb/c mice were purchased from Koatech Co. (Pyungtaek, Republic of Korea) and maintained under specific pathogen-free conditions. All animal experimental procedures were performed in accordance with the Guide for the Care and Use of Laboratory Animals. The study included 30 male Balb/c mice (mean age: 9 weeks, range: 8 to 10 weeks). For the time point study (experiment 1), mice were divided into 5 groups, with 3 mice per group. Mice were given a single administration of PLAG orally (2500 mg/kg BW) in a volume of 150 μL. And then mice are sacrificed after various time intervals, 0, 15, 30 and 60 minutes. To determine the difference in PLAG concentration (experiment 2), mice were divided into 5 groups, with 3 mice per group. They were given a single administration of PLAG orally (50, 250, 500, and 2500 mg/kg BW) in a volume of 150 μL, then sacrificed after 1 h post-injection.

The blood sample was obtained under anesthesia by heart puncture. Serum samples were separated by centrifuge 3,000 rpm for 10 minutes and stored at −80° C. for analysis. Lymph fluid from the cisterna chyli was collected according to the methods described by Masayuki, lymph fluid in cisterna chyli appears milky white color with a high concentration of triglyceride (TG) and located along the abdominal vena cava and aorta on the cranial side of the renal vein. A 30 ½-gauge syringe was carefully inserted into cisterna chyli. Then recovered the lymphatic fluid, diluted liquid with 10 uL of phosphate-buffered saline (Welgene, Gyeongsangbuk-do, Republic of Korea). Diluted lymph fluid samples were kept frozen for assay.

Triglyceride in blood and lymph fluid was measured using a commercial assay kit (Wako Diagnostics, Osaka, Japan). Because PLAG has structural similarities to TGs containing glycerol backbone, this method may be applicable to PLAG detection. Multi calibrator lipids (Wako) were used for standard reagent. The absorbance was read at 650 nm using an ELISA microplate reader (Molecular Devices Corporation).

Quantification of PLAG in blood and lymph fluid was performed in Mitsubishi Chemical Medicine Corporation (Japan) by radioactivity analysis. [¹⁴C] PLAG was synthesized by the LSI Medience Corporation (Kumamoto, Japan). The specific activities were 348.9 kBq/mg. The purity of the compound was 99.7%. The pharmacokinetics of [¹⁴C] PLAG were determined in Crl: CD (SD) rats (Charles River Laboratories Japan, Inc.). Rats received [¹⁴C] PLAG at 100 mg/5 mL/kg (dosing radioactivity: 5 MBq/kg) via the oral route. To determine radioactivity concentration in lymph fluid, the abdominal large thoracic duct-cannulated animals were fitted with collars while under isoflurane anesthesia and were subjected to the administration at least 30 minutes after collar fitting. Immediately after administration, the animals were set on a free moving apparatus. The largest total lymph fluid volume was selected for evaluation. Each of the collected samples was measured for radioactivity. Blood was collected from the subclavian vein and used to determine the radioactivity. Sampling time points are 0.5, 1, 2, 3, 4, 6, 8 and 24 h after administration. Radioactivity was measured by LSC (Tri-Carb 2300TR. Perkin Elmer, Inc) with the tSIE (transformed spectral index of external standard) method for the quenching correction.

Results

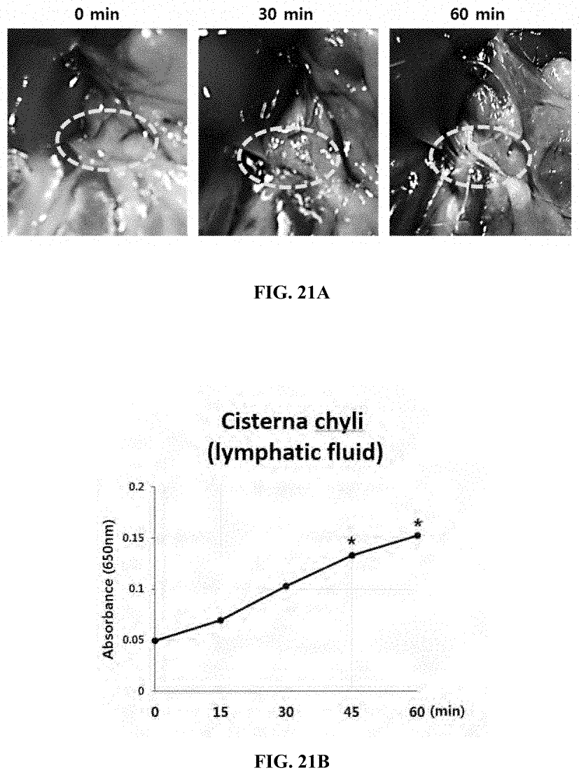

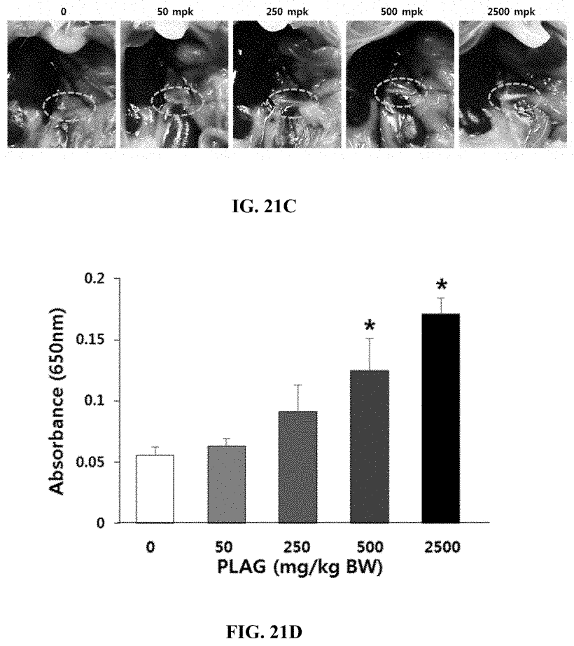

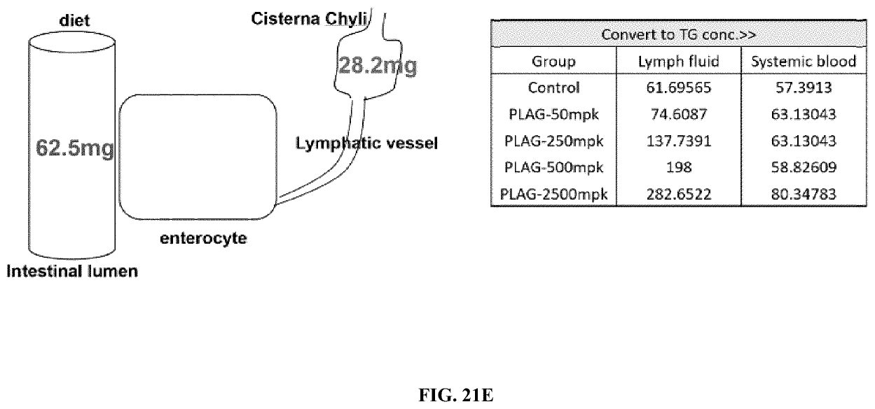

Synthesized chylomicron in enterocyte moves through the lymphatic vessel and toward cisterna chyli, subclavian vein and joins into a blood vessel. Cisterna chyli, temporal reservoirs of lymphatic fluid, contains chylomicron from enterocytes. PLAG, diacylglyceride, might be a component of the membrane of chylomicron. In fasting mice for 6 hours, diet 2500mpk of PLAG was detected in cisterna chyli at 60 minutes (FIG. 21A). The amount of PLAG discovered in cisterna chyli was further quantitatively confirmed at 0, 14, 30, 45 and 60 minutes using absorbance (FIG. 21B). In 50, 250, 500, 2500mpk of PLAG-fed mice, absorbed PLAG through intestinal epithelial cells was observed at the cisterna chyli within 1 hour in a dose-dependent manner (FIG. 21C). The amount of PLAG was further quantitatively confirmed using absorbance (FIG. 21D).

2500mpk of PLAG was orally administered to diet mice whose body weight is 25 g, absorbed PLAG was collected from cisterna chyli at 1 hr. Absorbed PLAG was evaluated by TG colorimetric assay. The TG concentration was multiplied by the dilution factor and the conversion factor, which is the coefficient required to convert to triglyceride concentration to the PLAG concentration. Administered 62.5 mg of PLAG was absorbed through enterocytes and detected 28.2 mg of PLAG at cisterna chyli within 1 hour with about 50% of absorptive efficacy (FIG. 21E). These data collectively confirm the PLAG uptake in the cisterna chyli with about 50% of absorptive efficiency.

### Example 22 Quantification of PLAG in Blood and Lymph Fluid

Materials and Methods

Quantification of PLAG in blood and lymph fluid was performed in Mitsubishi Chemical Medicine Corporation (Japan) by radioactivity analysis. [¹⁴C] PLAG was synthesized by the LSI Medience Corporation (Kumamoto, Japan). The specific activities were 348.9 kBq/mg. The purity of the compound was 99.7%. The pharmacokinetics of [¹⁴C] PLAG were determined in Crl: CD (SD) rats (Charles River Laboratories Japan, Inc.). Rats received [¹⁴C] PLAG at 100 mg/5 mL/kg (dosing radioactivity: 5 MBq/kg) via the oral route. To determine radioactivity concentration in lymph fluid, the abdominal large thoracic duct-cannulated animals were fitted with collars while under isoflurane anesthesia and were subjected to the administration at least 30 minutes after collar fitting. Immediately after administration, the animals were set on a free moving apparatus. The largest total lymph fluid volume was selected for evaluation. Each of the collected samples was measured for radioactivity. Blood was collected from the subclavian vein and used to determine the radioactivity. Sampling time points are 0.5, 1, 2, 3, 4, 6, 8 and 24 hours after administration. Radioactivity was measured by LSC (Tri-Carb 2300TR. Perkin Elmer, Inc) with the tSIE (transformed spectral index of external standard) method for the quenching correction.

Results

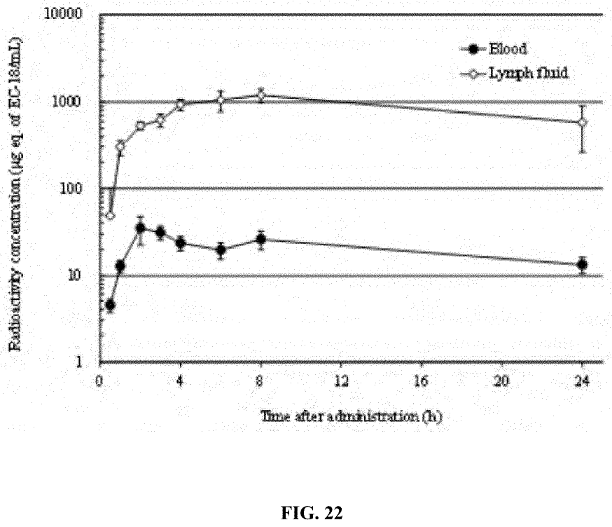

This study was performed to examine the radioactivity concentrations in blood and lymph fluid after a single oral administration of [14C] PLAG at a dose of 200 mg/kg in rats. The change of [¹⁴C] PLAG in blood and lymph fluid by time course is shown (FIG. 22). The concentration of PLAG gradually increased immediately following administration and reached the highest concentration at 8 hours. More PLAG was detected in the lymphatic vessel than blood vessel as shown in the figure. These results indicate that absorbed PLAG transfer to tissues through the lymphatic vessel, which is the same as the lipid absorption pathway, due to its structural characteristics.

### Example 23 Whole-Body Autoradiography of Mice after Single Oral Administration of PLAG

Materials and Methods

Tissue distribution of the PLAG after single oral administration of [¹⁴C] PLAG was conducted in Biotoxtech (Korea) using whole-body autoradiography. Crl:CD (SD) strain albino rats received the single oral administration of [¹⁴C] PLAG at a dose of 200 mg/kg and the animals were sacrificed by CO2 asphyxiation at defined times, and whole body autoradiograms were prepared to investigate the tissue distribution of radioactivity and its time course. Sampling time points are 15 minutes, 1 hour, 8 hours, and 24 hours after administration. The carcass was sliced into 40 micro meter-thick coronal plane sections that were collected on adhesive tape (NA-70, Nakagawa). The freeze-dried sections covered with a protective membrane were placed in contact with imaging plate (BAS-SR2025, Fuji Photo Film) and the plates were exposed in lead-sealed boxes at room temperature for 24 hours. After exposure, the radioactivity recorded on the imaging plate was analyzed using a bio-imaging analyzer system.

Results

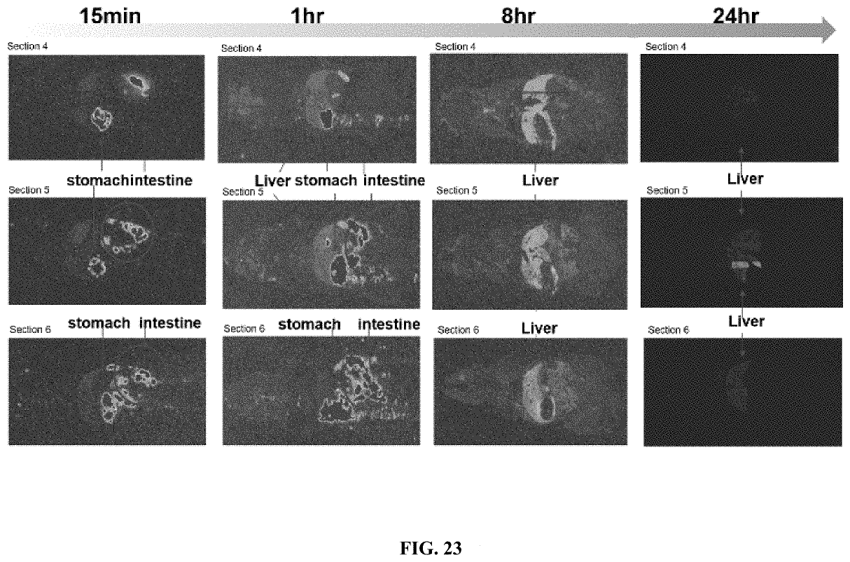

To investigate the tissue distribution of the PLAG, whole body autoradiography was performed after a single oral administration of [¹⁴C] PLAG at a dose of 200 mg/kg in rats (FIG. 23). As shown in the figure, high levels of radioactivity were observed in the stomach and the intestinal tract, including their luminal contents 15 minutes after administration and remained high levels of radioactivity until 1 hour after administration. Low levels of radioactivity were found in the liver at this time. 8 hours after administration, high levels of radioactivity were found in the luminal side of the stomach. Moderate levels of radioactivity were observed in the liver and brown fat tissues and low levels of radioactivity were found in the liver 24 hours after administration. However, the levels of radioactivity were not different from background level in any sampling time points in other tissues. These results suggest that PLAG as a lipid-like molecules is absorbed through the gastrointestinal tract and then distributed to liver and fat tissues.

### Example 24 Cumulative Excretion of Radioactivity after Single Oral Administration of PLAG

Materials and Methods

Excretion of PLAG at a single oral administration of 50 mg/kg BW to rats was examined in Mitsubishi Chemical Medicine Corporation (Japan) by radioactivity analysis. After oral administration of a single dose of 50 mg/kg, animals were individually accommodated in glass metabolic cages (Metabolica Model MC-CO2). Spontaneously excreted urine, feces, and radioactive molecules expired from each animal were collected. Sampling time points are 0-24, 24-48, 48-72 hours after administration. Then radioactivity and the excretion ratios and amount of radioactivity were determined. Radioactivity was measured by LSC (Tri-Carb 2300TR. Perkin Elmer, Inc) with the tSIE (transformed spectral index of external standard) method for the quenching correction.

Results

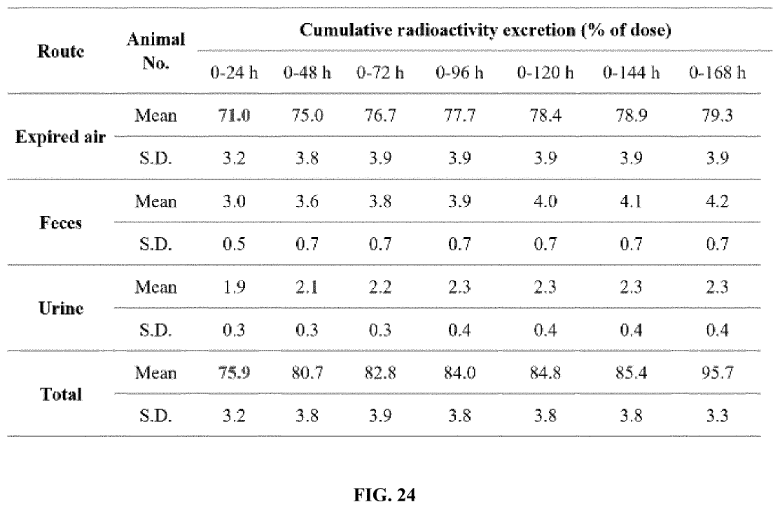

The cumulative amount of PLAG excretion via multiple routes such as urine, feces, and expired air is presented (FIG. 24). By 24 hours after oral administration, 1.9%, 3.0% and 71.0% of the dosed radioactivity were excreted into urine, feces, and expired air, respectively. The total recovery of radioactivity was 75.9% of the dosed radioactivity. These data suggest that exhaustion through expired air by lung is a major pathway of PLAG excretion, and approximately 76% of PLAG administered might be degraded and metabolized within 24 hours.

### Example 25 Vesicle/Micelle Formation of PLAG

Methods and Materials

Transmission Electron Microscopy (TEM)

The formation of micelles composed of PLAG or POPC was prepared in RPMI1640 medium by vigorous stirring at a final concentration of 10 mg/ml. The particle size of PLAG and POPC micelles was measured by dynamic light scattering (Zetasizer 3000HS, Malvern Instruments Ltd., UK). The morphological examination of the micelles was performed by transmission electron microscopy (TEM) by dropping the samples into the carbon films on the copper grid for viewing with 2% (weight per volume) phosphotungstic acid staining.

Results

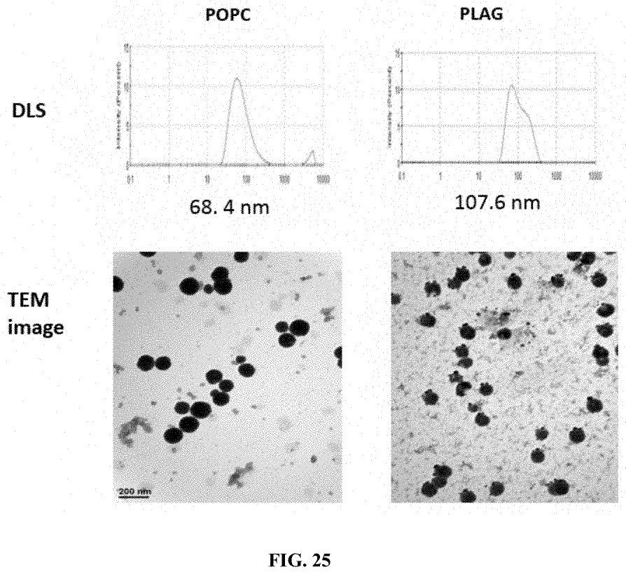

The micelle form is a way to transport lipids with hydrophobic characteristics through the circulating vessels. PLAG enables to form the micelles through hydrophobic interaction. The prediction structure of PLAG represents by figure. To confirm the particle size of PLAG, PLAG was vigorously agitated in the water until micelle formation. The average particle size and size distribution of the PLAG determined by dynamic light scattering (DLS) instrument are shown (FIG. 25, DLS). PLAG had an average 107.6 nm diameter. Transmission electron microscopy confirmed the determined diameter and showed that the particles had a spherical shape (FIG. 25, TEM image). As a positive control for micelle construction, phosphatidylcholine (POPC) was used. POPC is a class of phospholipids and well known as a major component of the cell membrane. These results indicate that PLAG functions in the form of micelles.

### Example 26 Biological Activity of PLAG is Dependent on LPL and GPIHBP1

Materials and Methods

In Vitro Phagocytosis and Bacterial Killing Assay

For immunofluorescence-based measurement of phagocytosis and clearance, BMDMs were grown on glass coverslips in 24-well plates. The cells were infected with PAK (MOI, 50) for different time intervals and then were treated with 10 μg/ml gentamycin for 30 minutes to remove extracellular PAK attached to the cell surface. The infected cells were washed with ice-cold PBS several times, followed by fixing for 10 minutes at room temperature in methanol or 10% paraformaldehyde. The cells were stained with anti-Pseudomonas antibody (Abcam) and then were incubated with goat anti-rabbit IgG secondary antibody conjugated with Alexa Fluor 488. Slides were mounted using the mounting medium ProLong™ Gold antifade reagent with DAPI (Thermo Scientific™) and were imaged with a confocal microscope (Zeiss LSM 800, Germany).

For measurement of phagocytosis by flow cytometry, PAK was heat-killed and stained with 10 μM of SYTO9 (Thermo Scientific™) at room temperature for 30 minutes and then was extensively washed with ice-cold PBS several times. The staining dose of PAK was determined by flow cytometry. THP-1 cells were infected with heat-killed and SYTO9-stained PAK (MOI, 50) for different time intervals at 37° C., after which they were washed with ice-cold PBS several times. The fluorescence of extracellular PAK attached to the cell surface was quenched by replacing the medium with PBS containing 0.2% trypan blue.

For CFU counting-based phagocytosis and bacterial killing assay, PAK was cultured at 37° C. overnight with continuous shaking and was resuspended in PBS. The BMDMs or THP-1 cells were incubated with PAK (MOI, 50) for different time intervals at 37° C. The cells were further cultured in the medium containing 10 μg/ml gentamycin for 30 minutes and then were lysed by 0.5% SDS. The diluted aliquots were spread on LB agar plates, and CFU was counted after incubation of the plates overnight at 37° C.

Results

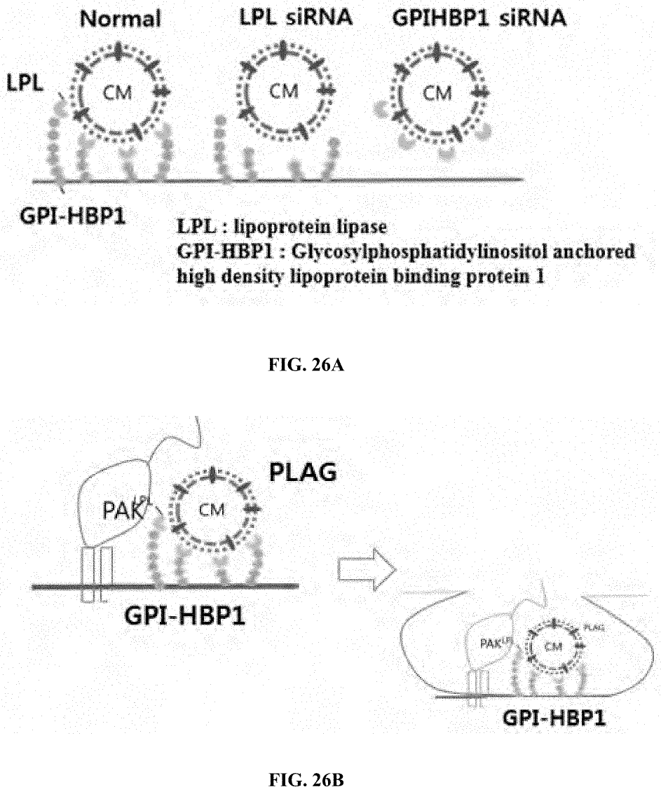

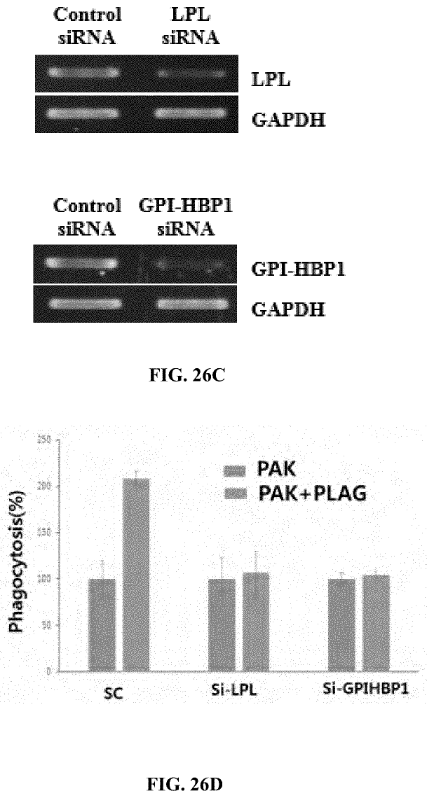

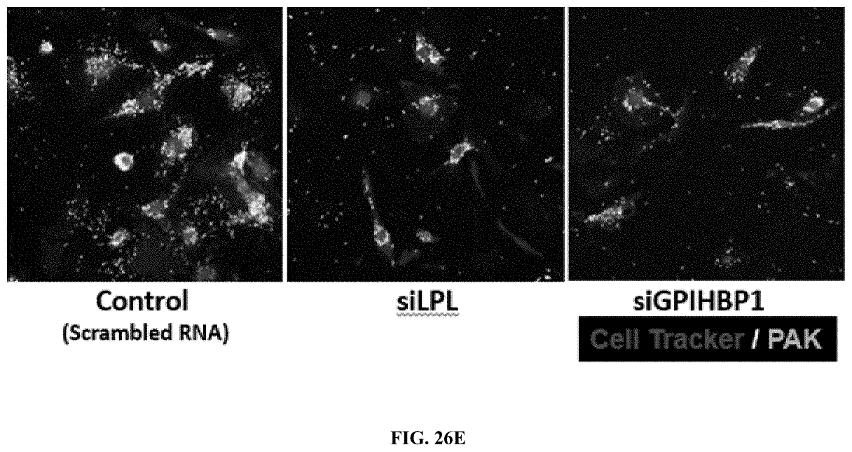

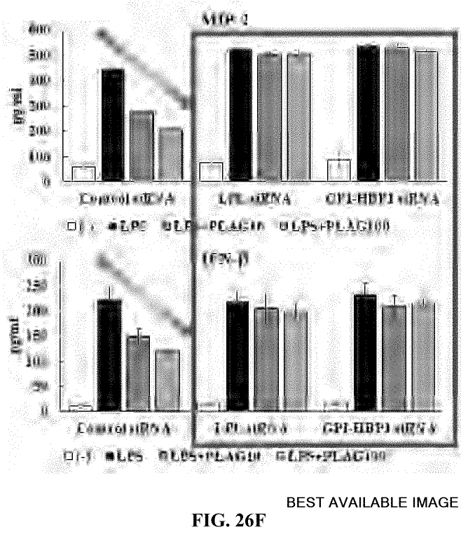

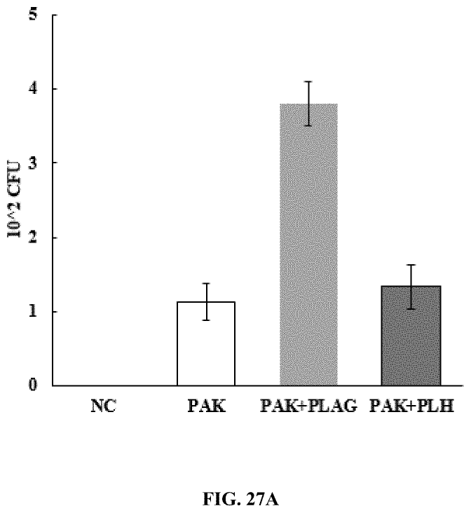

PLAG contacted with the cells in the form of micelles via the micelle-related ligands, LPL and GPIHBP-1. Thus, it was hypothesized that the effect of PLAG on the advanced phagocytosis and elimination of PAK by macrophages was also dependent on the same receptors. Target cells release LPL, which binds to micelle surface, and LPL-bound chylomicron is captured by GPIHBP1 (FIG. 26A). It was further hypothesized that the chylomicron captured by GPI-HBP1 and LPL promotes the phagocytosis of PAK (FIG. 26B). To test the hypothesis, LDL and/or GPIHBP-1 knockdown cells were prepared by transiently silencing these genes using siRNAs. LPL or GPIHBP1 gene silencing is carried out through the treatment of micro-RNA of LPL or GPIHBP1, as shown by the RT-PCR assessment (FIG. 26C). To investigate how LPL or GPIHBP-1 affects PAK phagocytosis and PLAG effect, in vitro phagocytosis assay was performed and measured the number of intracellular PAK at 1 h after infection in LPL or GPIHBP-1 silenced cells. It was observed that the PLAG effect on the enhanced bacterial phagocytosis was abrogated either in LPL or GPIHBP-1 silenced cells from the phagocytosis rate and confocal microscopy of control, LPL silenced and GPIHBP-1 silenced cells (FIGS. 26D and 26E). As another biological activity of PLAG, PLAG effectively down-regulates chemokine MIP-2 and cytokine IFN-β in the LPS treated macrophage cells (FIG. 26F). In the LPL or GPIHBP1 silenced cell, PLAG was not capable of modulating chemokine MIP-2. Thes data indicate that the modulation of chemokine by PLAG is dependent on LPL and GPIHBP1.

### Example 27 Acetylated Glycerol is Critical in the Monoacetyl Diacylglycerol Mediated Phagocytosis

Materials and Methods

In Vitro Phagocytosis Assay

For the CFU counting-based phagocytosis assay, PAK was cultured at 37° C. overnight with continuous shaking and then resuspended in PBS. BMDMs were pretreated with 100 μg/ml of PLAG or PLH for 1 hour. The cells were infected with PAK (multiplicity of infection [MOI], 50) for 1 h, and then treated with 10 μg/ml gentamycin for 30 minutes to remove extracellular PAK attached to the cell surface. Then, the cells were lysed with 0.5% SDS and serially diluted in PBS to spread on LB agar plates. CFU counts were performed after overnight incubation at 37° C.

For the immunofluorescence-based measurement of phagocytosis and clearance, BMDMs were grown on glass coverslips in 24-well plates. The cells were infected with PAK (MOI, 50) for 1 hour and then treated with 10 μg/ml gentamycin for 30 minutes to remove extracellular PAK attached to the cell surface. The infected cells were washed several times with ice-cold PBS and then fixed for 10 minutes at room temperature in methanol or 10% paraformaldehyde. The cells were then incubated with anti-Pseudomonas primary and Alexa Fluor 488-conjugated goat anti-rabbit IgG secondary antibodies. The coverslips were mounted on slides with ProLong Gold antifade reagent with DAPI (Thermo Fisher Scientific) and imaged with a confocal microscope (LSM 800; Zeiss, Germany).

PAK Infection and Colony-Forming Unit (CFU) Determination

Pseudomonas aeruginosa (PAK) was grown overnight in LB broth at 37° C. with agitation, and then harvested by centrifugation at 13,000×g for 2 min. The pellet was diluted to yield 1×10⁵ colony-forming unit (CFU) per 20 μL of phosphate-buffered saline (PBS) as determined by the optical density at 600 nm. The diluted bacteria were administered to BALB/c mice by intranasal injections. Bronchoalveolar lavage fluid (BALF) samples were then collected 2 hours after infection and serially diluted 1:1,000-1:10,000 with PBS and incubated overnight at 37° C. on LB agar plates. The number of viable bacteria in BALF samples were determined by counting the numbers of colonies formed on the plates.

Results

In the PAK (Pseudomonas aeruginosa K) introduced mice, bacterial clearance activity of PLAG was evaluated in the bronchoalveolar lavage fluid (BALF). While PLAG showed the bacterial clearance activity, PLH didn't. This confirmed that the specificity of PLAG originates from the acetyl group.

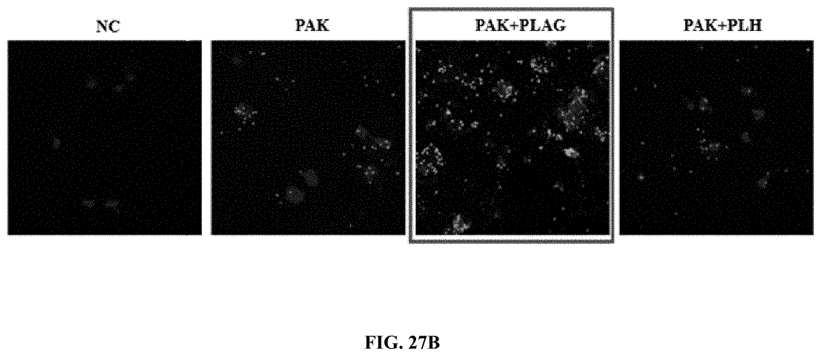

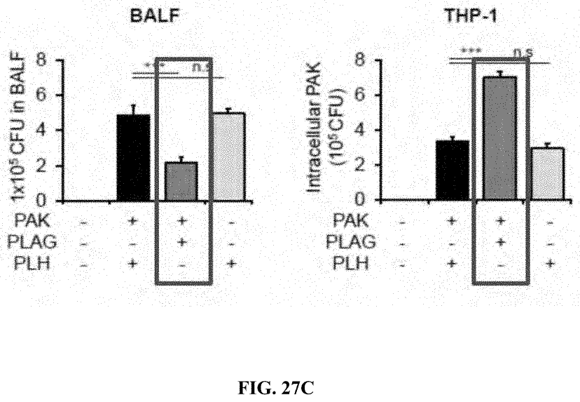

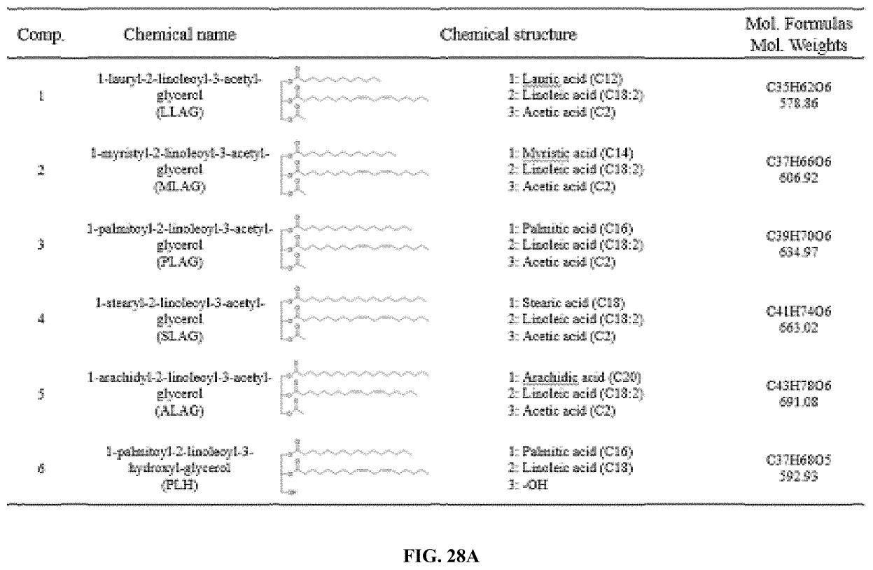

PLAG is a lipid molecule that has an acetyl group esterified at the third position of the glycerol backbone. It was investigated the uniqueness of PLAG in terms of bacterial phagocytosis and clearance by comparing PLAG with palmitic, linoleic hydroxyl glycerol (PLH). PLH is a form of diacylglycerol without an acetyl group. The specificity of PLAG in accelerating phagocytosis was compared with PLH. An acetylated micelle was hypothesized to be essential to accelerate phagocytosis. The hypothesis was confirmed as shown by the number of colony-forming units of the intracellular PAK and confocal microscopy of PAK treated cells, PAK/PLAG treated cells, and PAK/PLH treated cells (FIGS. 27A and 27B). In the PAK-induced pneumonia mice model, the PLAG treatment group effectively eliminated PAK in BALF, whereas the PLH treatment group did not (FIG. 27C, left). The effect of PLAG and PLH on phagocytosis of PAK by THP-1 cells was also compared 1 hour after infection (FIG. 27C, right). While PLAG showed the advanced phagocytosis of PAK, PLH had little effect on the bacterial internalization. The acetylated form in PLAG confers the efficacy of the bacterial phagocytosis and elimination.

### Example 28 PLAG is an Optimized Molecule for Biological Activities

Materials and Methods

In Vitro Phagocytosis Assay

For the CFU counting-based phagocytosis assay, PAK was cultured at 37° C. overnight with continuous shaking and then resuspended in PBS. BMDMs were pretreated with 100 μg/ml of LLAG, MLAG, PLAG, SLAG, or ALAG for 1 h. The cells were infected with PAK (multiplicity of infection [MOI], 50) for 1 h and then treated with 10 μg/ml gentamycin for 30 minutes to remove extracellular PAK attached to the cell surface. Then, the cells were lysed with 0.5% SDS and serially diluted in PBS to spread on LB agar plates. CFU counts were performed after overnight incubation at 37° C.

For the immunofluorescence-based measurement of phagocytosis and clearance, BMDMs were grown on glass coverslips in 24-well plates. The cells were infected with PAK (MOI, 50) for 1 h and then treated with 10 μg/ml gentamycin for 30 minutes to remove extracellular PAK attached to the cell surface. The infected cells were washed several times with ice-cold PBS and then fixed for 10 minutes at room temperature in methanol or 10% paraformaldehyde. The cells were then incubated with anti-Pseudomonas primary and Alexa Fluor 488-conjugated goat anti-rabbit IgG secondary antibodies. The coverslips were mounted on slides with ProLong Gold antifade reagent with DAPI (Thermo Fisher Scientific) and imaged with a confocal microscope (LSM 800; Zeiss, Germany).

Results

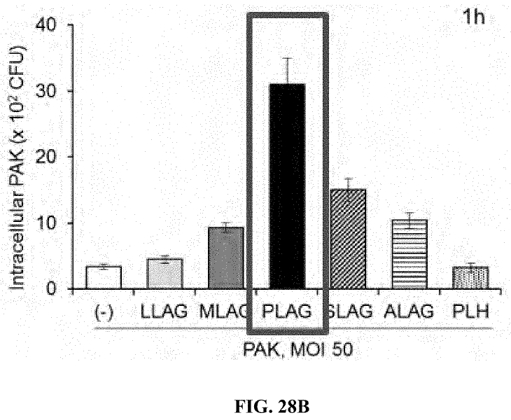

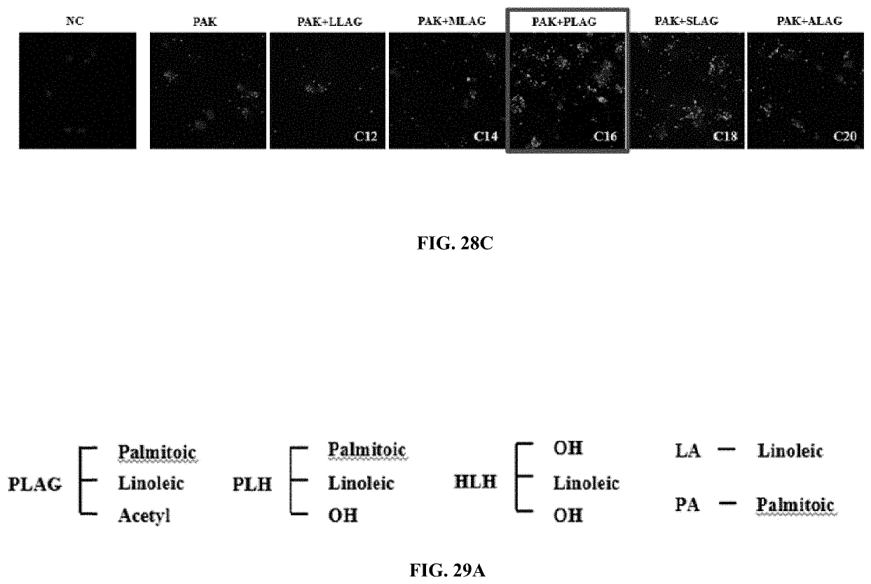

To investigate whether the length of fatty acids esterified at the first position of the glycerol backbone of the lipids contributes to the phagocytic activity of BMDMs, different sizes of fatty acid at 1-position were examined (FIG. 28A). Cells were pretreated with six glycerols LLAG, MLAG, PLAG, SLAG or ALAG and PLH for 1 hour and then infected with PAK (MOI, 50) for 1 hour. As a result, PLAG, which has palmitic acid (C16) esterified at the first position of the glycerol backbone, most effectively enhanced the phagocytic activity of BMDMs, as shown by the number of colony-forming units of the intracellular PAK and confocal microscopy of cells treated by six different glycerols (FIGS. 28B and 28C). These observations indicate that PLAG is an optimized molecule for enhancing the phagocytic activity of macrophages.

### Example 29 Comparison of PLAG with Other Monoacetyl Diacylglycerols in LPS Induced Acute Lung Injury (ALI)

Materials and Methods

ALI Model and Materials

Balb/c mice (9-week to 11-week-old males) were purchased from Koatech Co. (Pyongtaek, Republic of Korea) and maintained under specific pathogen-free (SPF) conditions. All animal studies were performed in accordance with the Guide and Use of Laboratory Animals (Institute of Laboratory Animal Resources). All experiments were approved by the Institutional Review Committee for Animal Care and Use of KRIBB (Korea Research Institute of Bioscience and Biotechnology, Daejeon, Republic of Korea). The approval number is KRIBB-AEC-16031.

For the ALI model, mice were anesthetized with 150 mg/kg of 2,2,2-Tribromoethanol (Sigma Aldrich, St. Louis, Mo., USA)) by intraperitoneal injection and administered LPS intranasally (25 mg/kg, Sigma Aldrich). PLAG (250 mg/kg, Enzychem Lifesciences Co., Daejeon, Republic of Korea) was administered orally. The collection of bronchoalveolar lavage fluid (BALF) was performed by tracheal cannulation using cold phosphate-buffered saline (PBS). A complete blood count (CBC) was performed using the Mindray BC-5300 auto hematology analyzer (Shenzhen Mindray Bio-medical Electronics, China).

Immunofluorescence Staining and Flow Cytometric Analysis

To detect TLR4/MD2 on the membrane surface, cells were fixed with 2% paraformaldehyde (Sigma-Aldrich) and were blocked with PBS containing 1% BSA (Gibco, Waltham, Mass., USA). They were incubated with rabbit anti-TLR4/MD2 antibody (Thermo) and Alexa488 conjugated anti-rabbit IgG (Invitrogen). For confocal microscopy analysis, cells were washed with PBS and mounted in DAPI-containing fluorescence microscopy mounting medium (Invitrogen). Samples were analyzed with a laser scanning confocal microscope (Carl Zeiss, Oberkochen, Germany). For flow cytometric analysis, cells were washed and analyzed with a FACSVerse flow cytometer (BD Biosciences), and data were processed with FlowJo software (Tree Star, OR, USA).

Results

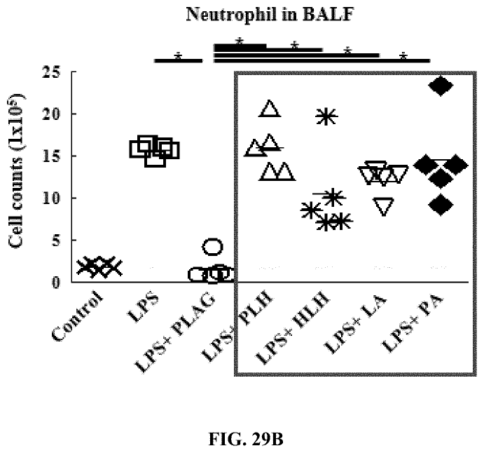

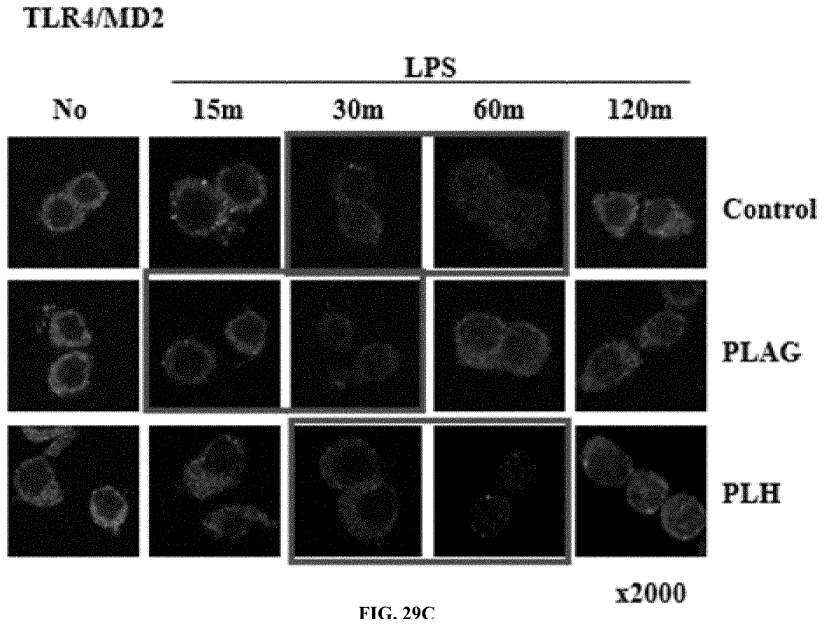

To determine the specificity of PLAG, an acetylated DAG, the therapeutic efficacy of PLAG metabolites was assessed and compared to their biological efficacy in the animal model in vivo (FIG. 29A). PLH is a DAG that consists of two fatty acid chains, palmitic acid and linoleic acid. HLH is composed of linoleic acid and a glycerol backbone. Linoleic acid (LA) or palmitic acid (PA) was also used. In the ALI animal model, LPS treatment via intranasal administration induces massive neutrophil extravasation into the alveolar cavity, which is easily detected in the BALF. PLAG co-treated mice show a dramatically reduced number of neutrophils in the BALF and counts rapidly return to a normal status. Conversely, PLH, HLH, LA, and PA have no effect on the number of neutrophils in BALF from LPS-treated mice (FIG. 29B). These data indicate that PLAG has a specific role in blocking the excessive and sustained neutrophil infiltration during LPS-induced ALI progression. In LPS treated cells, TLR4/MD2 initiated internalization at about 30 minutes and returned to the surface at about 120 minutes. In contrast, in LPS/PLAG treated cells, TLR4/MD2 initiated internalization at about 15 minutes and returned to the surface at about 60 minutes. However, LPS/PLH had no effect on TLR4/MD2 internalization and returned to the surface, compared to LPS treated cells (FIG. 29C). These findings suggest that the acetylation of DAG is a critical factor in blocking excessive neutrophil extravasation and accelerating phagocytosis in the ALI animal model.

### Example 30 PLAG Promotes the Uptake of Triglyceride (TG) at Peripheral Tissues in the STZ-Induced Mice Model

Materials and Methods

Eight-week-old Balb/c male mice were purchased from Koatech Co. (Pyeongtaek, Republic of Korea) and maintained in for 7 days in order to adapt to the environment. The experimental protocol was approved by the Animal Care and Use Committee of Korea Research Institute of Bioscience and Biotechnology Institution (KRIBB-AEC-17146) and performed in accordance with the National Institutes of Health Guidelines for the care and use of laboratory animals and with the Korean national laws for animal welfare. The mice were randomized initially into two experimental groups as follows: Non-treated group as a control; STZ treated group. STZ treated groups received an intraperitoneal injection of 200 mg/kg BW STZ, which was dissolved in citrate buffer (pH 4.5), while the animals belonging to the control group received vehicle injection. The next day following STZ administration, the induction of diabetes in all STZ-treated mice was confirmed the glucose level in blood by a glucometer (ACCU-CHEK, Roche diagnostics Inc., Seoul, Korea). All mice with blood glucose levels higher than 200 mg/dL in fasting state were considered acute diabetes. After the confirmation of the induction of diabetes, the mice of the experimental group were further randomized into three groups: STZ alone treated group; high dose of PLAG treated group; a low dose of PLAG treated group. PLAG was injected into mice for 3 days. Control and STZ alone treated groups were administrated orally to mice with the same PBS for 3 days.

Following sacrifice, LPL activity was measured in plasma using a quantitative LPL activity assay kit (Cell Biolabs, Inc, San Diego, Calif.). Diluted samples and standards were loaded to the fluorescence microtiter plate, and LPL fluorometric substrate was added. After 30 minutes, the reaction stopped by stopping the solution. After 15 minutes, the sample fluorescence measured by a fluorescence microplate reader.

The relative amounts of Apolipoprotein B48 (ApoB48) of portal vein plasma were evaluated by western blot analysis. Constant volumes of plasma were separated on 5% SDS-PAG. The protein extracts were immunoblotted with the ApoB48 antibody (Abcam, MA, USA).

Results

Lipid uptake through chylomicron was examined in the diabetes model. Uptake lipid forms chylomicron (CM) and absorbed in the peripheral tissue with the aid of LPL. Unabsorbed lipids remaining in CM remnant move to the liver via the portal vein. Streptozotocin (STZ) is generally used for the induction of diabetes. STZ down-regulates insulin and LPL (Lipoprotein lipase). CM is recognized by LPL. LPL downregulated peripheral tissue is unable to access to CM and lipid uptake was severally inhibited in the STZ treated mice. PLAG localized in the membrane of CM and recognized by SR-A (scavenger receptor A, CD204) which gives a chance to contact CM and tissue and successively make lipid-uptake.

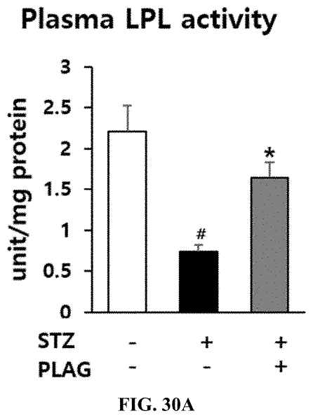

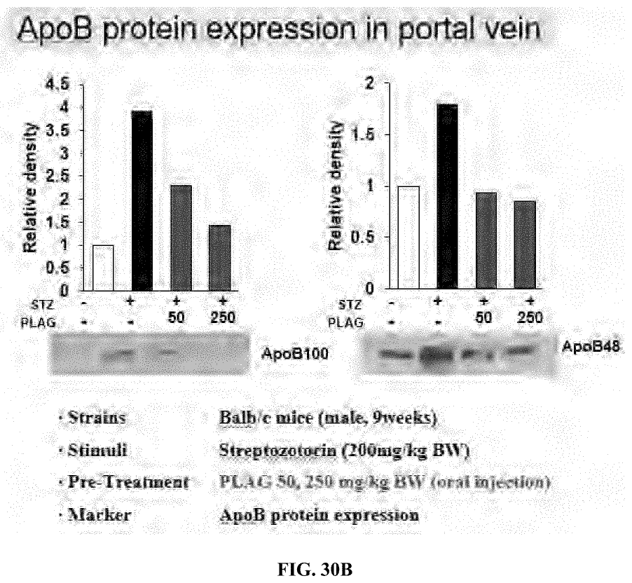

Lipoprotein lipase (LPL), the rate-limiting enzyme in TG clearance, controls catabolism of TG-rich lipoproteins, including CM. To investigate TG clearance following PLAG treatment by LPL activity, an LPL activity assay was conducted in plasma. The plasma LPL activity was significantly decreased in the streptozotocin (STZ) group, while recovered in PLAG treated group (FIG. 30A).

Apolipoprotein B48 (ApoB48) is composed of CM and as a marker of TG-rich CM transport and uptake in the body. Increased ApoB48 in portal vein might consider that insufficient TG uptake into peripheral tissues or overall increased systemic TG by hepatic steatosis. In this study, ApoB48 levels increased in portal vein blood in the STZ group, and PLAG treatment markedly reduced the ApoB48 level in a dose-dependent manner (FIG. 30B). These results indicate that PLAG improved lipid metabolism in hepatic steatosis by promoting TG uptake to peripheral tissue.

### Example 31 PLAG Dose-Dependently Alleviates an Accumulation of Triglyceride in the Liver

Materials and Methods

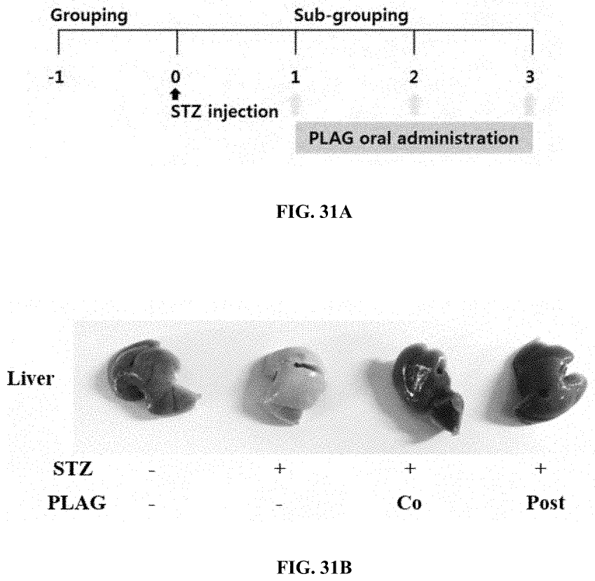

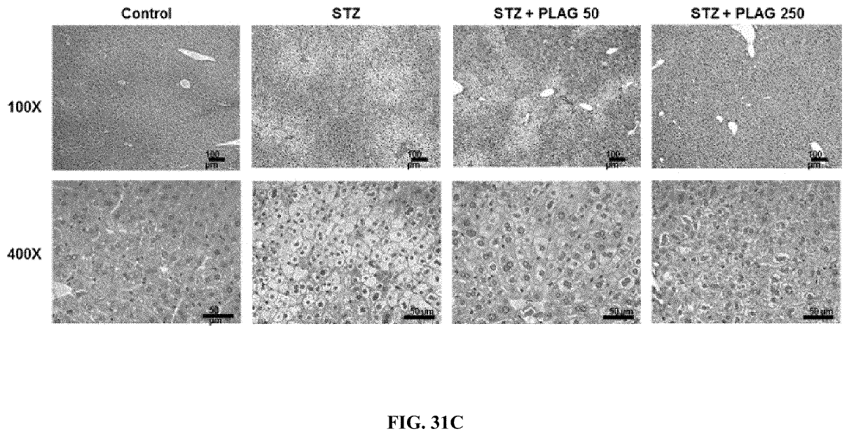

Eight-week-old Balb/c male mice were purchased from Koatech Co. (Pyeongtaek, Republic of Korea) and maintained in for 7 days in order to adapt to the environment. The experimental protocol was approved by the Animal Care and Use Committee of Korea Research Institute of Bioscience and Biotechnology Institution (KRIBB-AEC-17146) and performed in accordance with the National Institutes of Health Guidelines for the care and use of laboratory animals and with the Korean national laws for animal welfare. The mice were randomized initially into 2 experimental groups as follows: Non-treated group as a control; STZ treated group. STZ treated groups received an intraperitoneal injection of 200 mg/kg BW STZ, which was dissolved in citrate buffer (pH 4.5), while the animals belonging to the control group received vehicle injection. The next day following STZ administration, the induction of diabetes in all STZ-treated mice was confirmed the glucose level in blood by a glucometer (ACCU-CHEK, Roche diagnostics Inc., Seoul, Korea). All mice with blood glucose levels higher than 200 mg/dL in fasting state were considered acute diabetes. After the confirmation of the induction of diabetes, the mice of the experimental group were further randomized into three groups: STZ alone treated group; high dose of PLAG treated group; a low dose of PLAG treated group. One day after STZ injection, mice were orally injected with PLAG for 3 days (FIG. 31A). The dosage and preparation of PLAG were determined according to previous reports. Control and STZ alone treated groups were administrated orally with the same PBS for 3 days.

The experiments were carried out according to the methods mentioned above. Following sacrifice, the liver was immediately fixed in 10% formalin at room temperature, and then the tissues were embedded in paraffin, sectioned and stained with hematoxylin and eosin (H&E).

Results

Treatment of streptozotocin (STZ) induced liver steatosis. The color of the liver of the STZ treated group was visually different from livers of the control, STZ and PLAG co- or post-treated group (FIG. 31B). To further confirm the extent of fat accumulation, H&E staining of the liver tissues was conducted. As expected, H&E staining showed that STZ treated group induced hepatic steatosis by confirming the increase of empty fat vacuoles (FIG. 31C). 250 mg/kg of PLAG decreased STZ-induced empty fat vacuoles more effectively than 50 mg/kg of PLAG.

### Example 32 PLAG Recovers the LPL Expression in Muscle Cells of STZ Treated Mice

Materials and Methods

Total RNA was isolated from the muscle of individual mice using TRIzol reagent (Favorgen Biotech, Taiwan). The cDNA was synthesized using M-MLV reverse transcriptase according to the manufacturer's instructions (Promega, Madison, Wis., USA). The gene expression from each sample was analyzed in duplicates and normalized against the internal control gene GAPDH. Sequences of used primers are as follows: GAPDH, forward 5′-CCATCACCATCTTCCAGGAG-3′ (SEQ ID NO. 7), reverse 5′-ACAGTCTTCTGGGTGGCAGT-3′ (SEQ ID NO. 8); LPL, forward 5′-GGGCTCTGCCTGAGTTGTAG-3′ (SEQ ID NO. 9), reverse 5′-GTCAGGCCAGCTGAAGTAGG-3′ (SEQ ID NO. 10).

For immunohistochemical staining, the deparaffinized tissues were treated with 3% hydrogen peroxide in methanol to quench the endogenous peroxidase activity, followed by blocking with 1% BSA. For the identification of LPL in muscle, the sections were incubated in the LPL antibody (1:100, Santa Cruz Biotechnology, Dallas, Tex.) at 4° C. overnight. The slides were then incubated with HRP-conjugated goat anti-mouse IgG (1:300, Santa Cruz Biotechnology) at room temperature for 15 minutes followed by visualization with the 3-amino-9-ethylcarbazole (AEC) substrate (Dako, Glostrup, Denmark). The tissues were stained with 10% Mayer's hematoxylin, dehydrated, and mounted using the Crystal Mount™ medium (Sigma-Aldrich). The images were obtained under light microscopy (Olympus, Tokyo, Japan).

The content of triglyceride in the skeletal muscle was examined by slightly modifying the method of Bose et al. Briefly, tissue was homogenized in isopropanol with a tissue grinder. The homogenate was centrifuged at 2,000 g for 10 minutes, and the supernatant was collected. The triglyceride content of the supernatant was measured by a Triglyceride H kit (Wako Diagnostics, Richmond, Va.).

Results

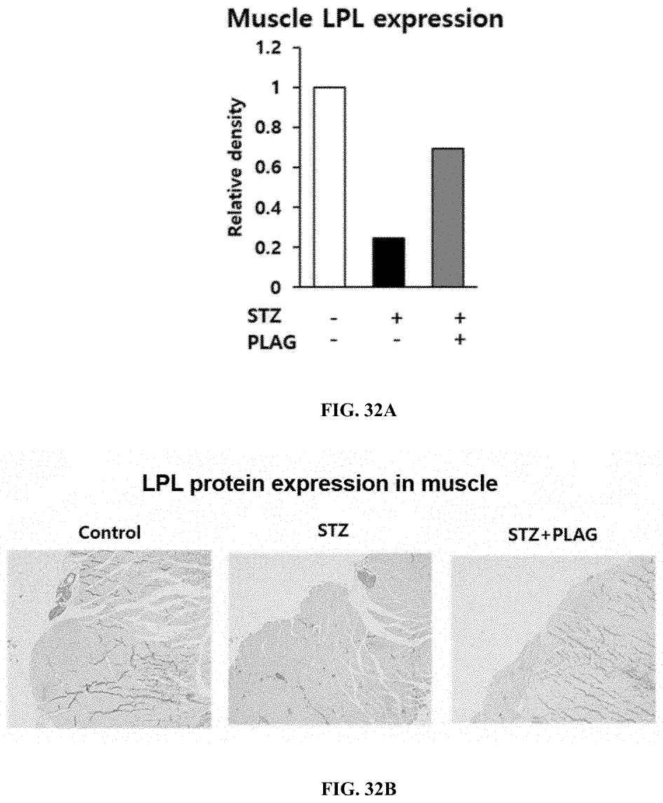

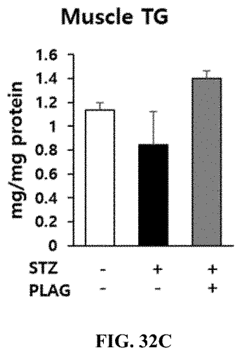

The RT-PCR results showed that mRNA expression of LPL of muscle cells was significantly reduced in the STZ group compared with the control group but not in the PLAG group (FIG. 32A). Immunohistochemistry staining was used to identify the presence of LPL in the muscle (FIG. 32B). The control group expressed LPL in a muscle compared to the STZ group. LPL protein expression in the PLAG treated group was similar to those control groups. These results were consistent with LPL mRNA expression in muscle. STZ treatment decreased muscle TG content which indicates that chylomicron remnant contains more TG. (FIG. 32C). Thus, TG in chylomicron delivered to liver increases in the STZ treated mice. In contrast, muscle TG content was recovered in the STZ/PLAG treated group. PLAG effectively reduced TG content in chylomicron delivered to the liver with the recovery of LPL activity. These data indicate that PLAG recovers the LPL expression in muscle cells of mice decreased by STZ treatment.

### Example 33, PLAG Attenuates STZ-Induced Hepatic Steatosis

Materials and Methods

Eight-week-old Balb/c male mice were purchased from Koatech Co. (Pyeongtaek, Republic of Korea) and maintained in for 7 days in order to adapt to the environment. The mice were randomized into four experimental groups as follows: Non-treated group as a control; STZ treated group; STZ and PLAG co-treated group; STZ and PLG co-treated group. Mice were orally injected with PLAG and PLG for 3 days. The dosage and preparation of PLAG were determined according to previous reports. Control and STZ alone treated groups were administrated orally with the same PBS for 3 days. Before the sacrifice, body weight was measured, and H&E staining was performed to confirm the histologic presence of hepatic steatosis.

Results

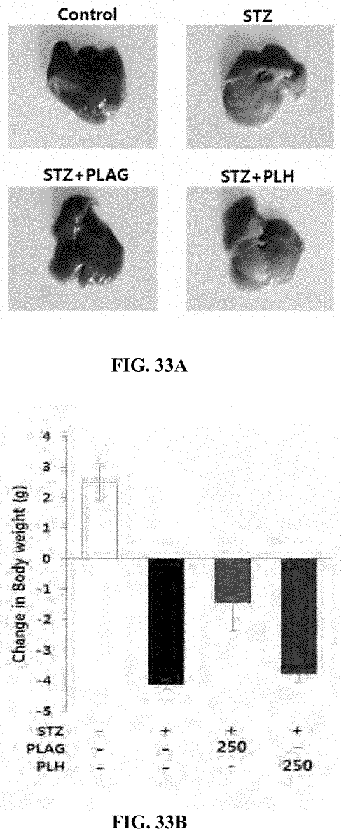

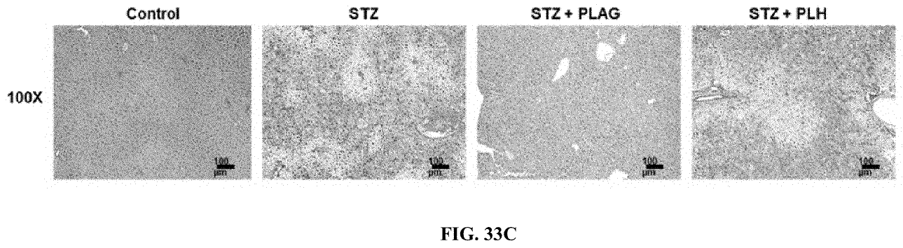

The selectivity of PLAG was confirmed by a comparison of PLAG and PLH in the STZ-induced mouse model. PLH is a prototype of DAG and PLAG is a type of acetylated DAG. The specificity of PLAG function in the alleviation of hepatic steatosis was examined. The color of the livers of the STZ treated group and STZ and PLH co-treated group was visually different from the livers of the control and STZ/PLAG treated group (FIG. 33A). This indicates that the control and STZ/PLAG treated mice showed hepatic steatosis induced by STZ. STZ and PLAG co-treated mice showed less body weight loss compared to STZ treated mice (FIG. 33B). There was no difference in the body weight between the STZ group and STZ and PLG co-treated mice. In addition, the representative histology results showed that treatment with STZ/PLAG attenuated hepatic steatosis (FIG. 33C). In contrast, liver damage was not altered by treatment with STZ/PLH. These results suggest that PLAG plays a unique role in the protection of the liver in STZ-induced hepatic steatosis.

### Example 34 PLAG does not Depend on CD36 in Reducing MSU Crystal-Induced CXCL8

Materials and Methods

The specific siRNA of CD36 (sc-29995) and control siRNA (sc-37007) were purchased from Santa Cruz Biotechnology. Cells were transfected with 50 nM of either the targeting or control siRNA using HiPerFect Transfection Reagent (Qiagen, Hilden, Germany) for 24 h. The knockdown efficiency of siRNAs was confirmed by Western blot analysis.

Transfected THP-1 cells were pre-incubated with PLAG (10, 100 μg/ml) for 1 hour and then stimulated with MSU crystal (400 μg/ml). After 24 hours, cells were centrifuged, and the supernatant was harvested. The concentrations of CXCL8 in the supernatant of THP-1 cells were measured using Human CXCL8 ELISA kit (BD bioscience, New Jersey, USA) according to the manufacturer's instructions. The cytokine levels were estimated by interpolation from a standard curve using an ELISA reader (Molecular Devices, Sunnyvale, USA) at 450 nm.

Results

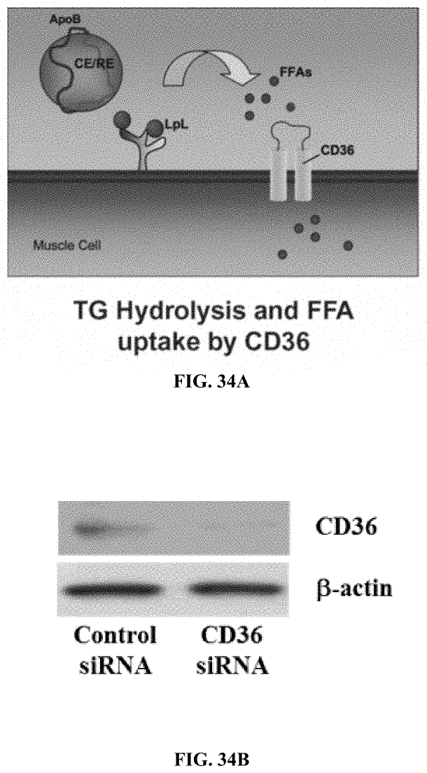

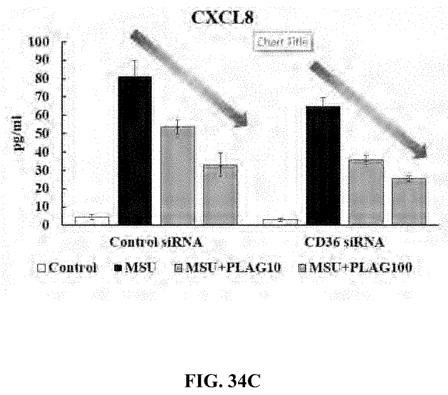

Lipid uptake through chylomicron is dependent on LPL and GPIHBP1. From trapped chylomicron (CM) by GPIHBP1, Free fatty acid (FFA) released by LPL is absorbed into target cells via CD36 receptor. (FIG. 34A). In order to determine whether PLAG acts as a vesicle or free fatty acid form, it was experimented with CD36 knockdown conditions using siRNA (FIG. 34B). If PLAG activity was originated from lipids of absorbed into target cells (including metabolites), there would be no biological activity of PLAG in the CD36 silenced cells. PLAG activity on chemokine modulation (i.e. decrease of CXCL8 induced by MSU crystal), however, is still shown in in the CD36 silenced cells (FIG. 34C). These data suggest that PLAG does not depend on CD36 and works as chylomicron, not metabolites in reducing MSU crystal-induced CXCL8.

### Example 35 PLAG is not Dependent on CD 36 in Modulating the Endocytosis of P2Y6 Receptor

Materials and Methods

The specific siRNA of CD36 (sc-29995) and control siRNA (sc-37007) were purchased from Santa Cruz Biotechnology. Cells were transfected with 50 nM of either the targeting or control siRNA using HiPerFect Transfection Reagent (Qiagen, Hilden, Germany) for 24 h. The knockdown efficiency of siRNAs was confirmed by Western blot analysis.

THP-1 cells were pre-incubated with PLAG (100 μg/ml) for 1 hour and then stimulated with MSU crystal (400 μg/ml). After 15, 30, 60 min, cells were harvested. To detect for the P2Y6 receptor on the membrane surface, cells were fixed with 4% paraformaldehyde (Sigma-Aldrich) and incubated with PBS containing 1% BSA for blocking. They were labeled with the rabbit anti-P2Y6 receptor antibody (1:200, APR-011, Alomone Labs, Jerusalem, Israel) for 1 h. The detection Ab was used with Alexa Flour 488 goat anti-rabbit IgG (Invitrogen). For flow cytometric analysis, cells were washed with 1% FBS/PBS and analyzed with a FACSVerse flow cytometer (BD Biosciences). FlowJo software (Tree Star, OR, USA) was used data processing.

Results

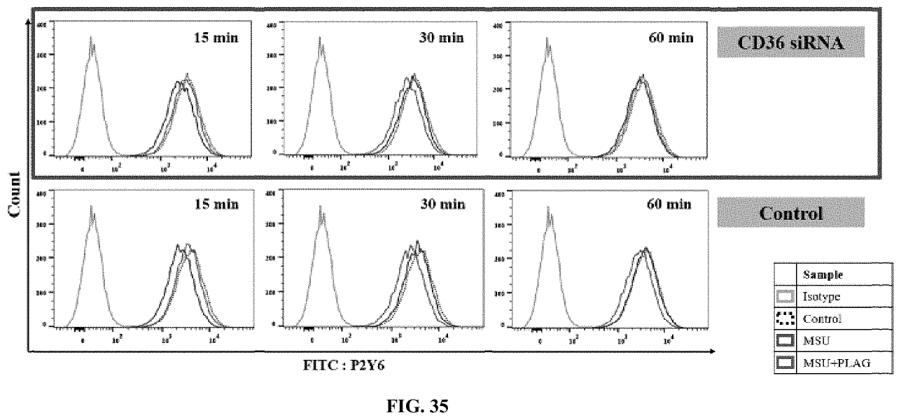

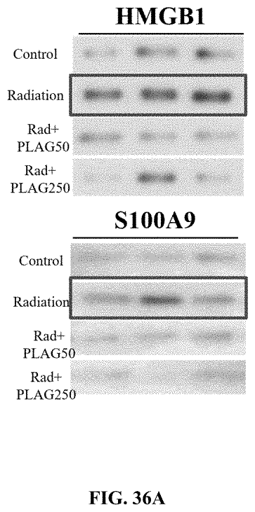

CD36 is a protein that transports free fatty acid into cells. In order to determine whether PLAG acts as a vesicle or free fatty acid form, it was experimented with CD36 knockdown conditions using siRNA. PLAG has the effect of promoting the endocytosis of the P2Y6 receptor which recognizes the MSU crystal, as shown by the flow cytometric analysis (FIG. 35, upper row). The promotion of P2Y6 receptor endocytosis was still observed under the silent condition of CD36 (FIG. 35, Lower row). These data suggest that PLAG does not depend on CD36 and works as chylomicron not metabolites in modulating the endocytosis of the P2Y6 receptor.

### Example 36 PLAG Modulates the Clearance of DAMP Molecules Induced by Radiation

Materials and Methods

The total-body of mice were irradiated with a gamma-ray of 6.11Gy on day 0. After that, the body weight of 1 day was measured and divided into 3 groups according to the average. PLAG (50, 250 mg/kg) or PBS was orally administered for 3 days from 1 day and sacrificed on the 3rd day. Add 3 ul serum to 97 ul of 1×SDS sample buffer and boiled for 10 min. Proteins from each sample were separated by sodium dodecyl sulfate-polyacrylamide gel electrophoresis on 8% polyacrylamide gels and the proteins were blotted onto a PVDF membrane (Millipore Corporation, Germany). The membrane was blocked with 5% non-fat dried milk (BD bioscience) in PBS containing 0.05% Tween-20 (Calbiochem) for 1 h. The membrane was incubated with anti-HMGB1 (abcam), anti-MRP14 (abcam) at 4° C. overnight. After washing with PBS containing 0.05% Tween-20, the membrane was stained with goat anti-rabbit IgG peroxidase (ENZO). Target proteins were detected with Immobilon Western Chemiluminescent HRP Substrate (Millipore Corporation).

Results

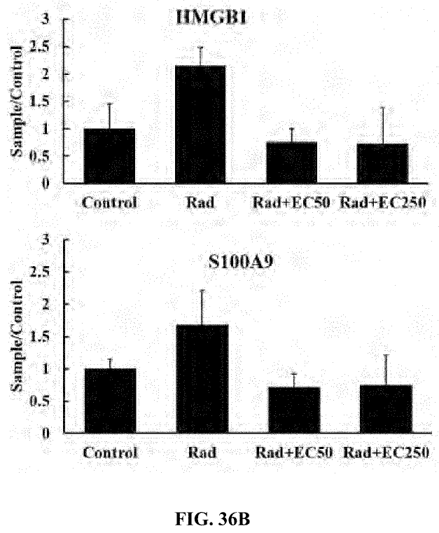

Radiation causes systemic tissue damage, which in turn induces the release of DAMP molecules such as HMGB1 and S100A9. Radiation-induced DAMP molecules were analyzed in the supernatant of radiated mice as shown by the western blot and relative gene expressions of HMGB1 and S100A9 after radiation, radiation/PLAG 50mpk treatment, and radiation/PLAG 250mpk treatment (FIGS. 36A and 36B). PLAG significantly decreased the level of HMGB1 and S100A9 induced by radiation in mice. These data suggest that PLAG modulates the clearance of DAMP molecules induced by radiation.

### Example 37 PLAG Attenuates Radiation-Induced Lung Injury in Mice

Materials and Methods

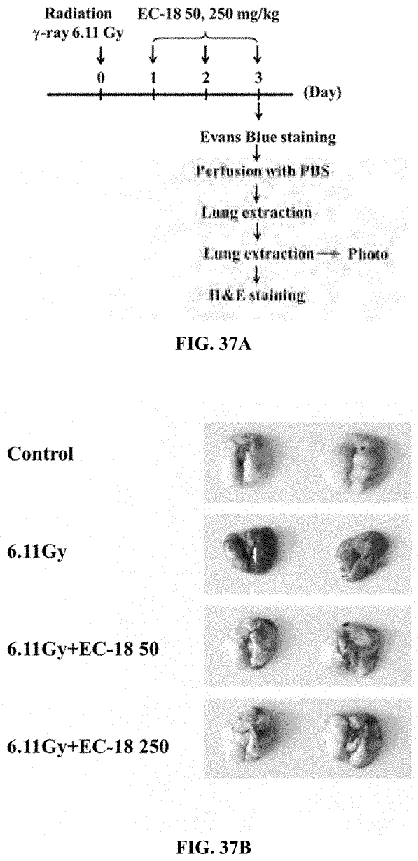

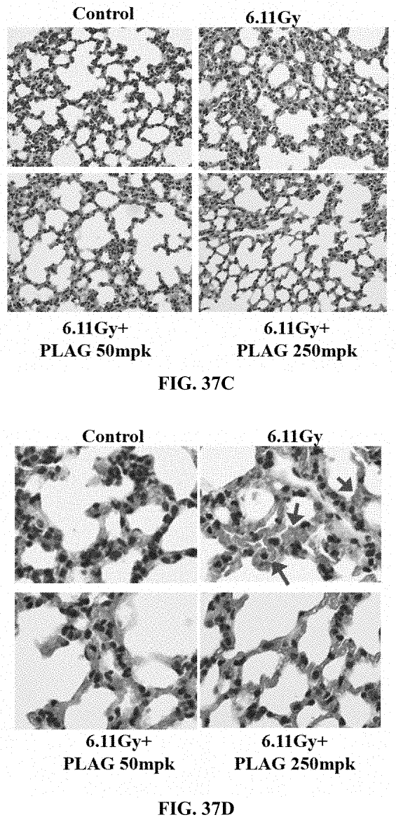

Balb/c mice were grouped into four groups: 1) control group, 2) radiation group, 3) radiation+EC-18 50 mg/kg of PLAG, and 4) radiation+EC-18 250 mg/kg of PLAG. Lung was extracted at day 3 after 6.11Gy of TBI by γ-ray (FIG. 37A). EC-18 was treated daily. Tissue was fixed with formaldehyde, and H&E staining was carried out.

Results

Pulmonary capillary leakage in the radiation treated mice was observed (FIG. 37B). Radiation/PLAG treated mice dose-dependently showed less pulmonary capillary leakage. H&E staining was further carried out to lung tissues of control, radiation-treated, radiation/PLAG 50mpk treated, and radiation/PLAG 250mpk treated mice (FIG. 37C). In the enlarged H&E stained lung tissues, leakage erythrocytes was observed at the interstitial tissue in the radiation treated mice (FIG. 37D, arrows). Leakage erythrocyte was not observed in the radiation/PLAG 50mpk and radiation/PLAG 250mpk treated mice. These data indicate that PLAG is capable of protecting the lung from tissue damage caused by lethal radiation.

### Example 38 PLAG Attenuates Skin Erythema Injury in Mice

Materials and Methods

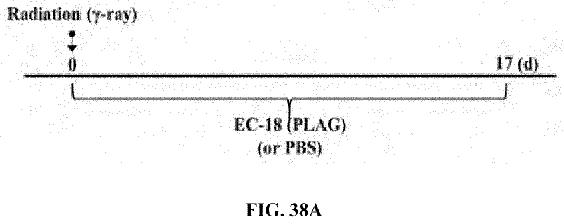

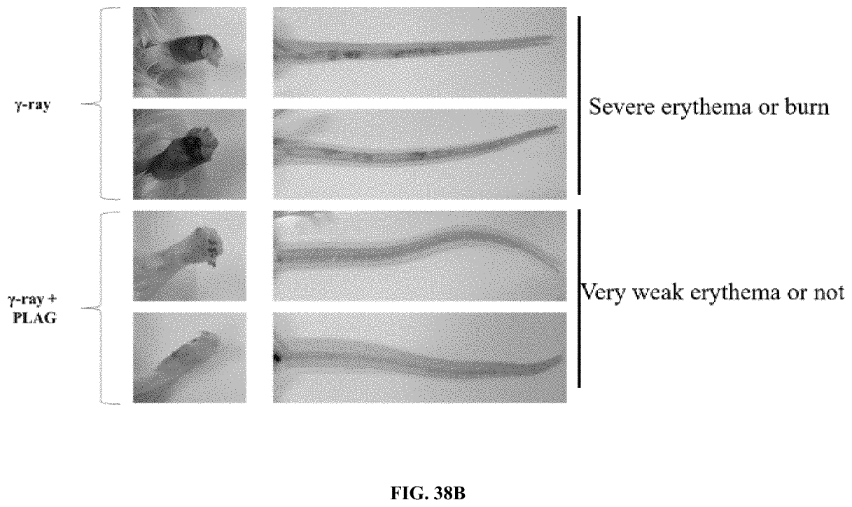

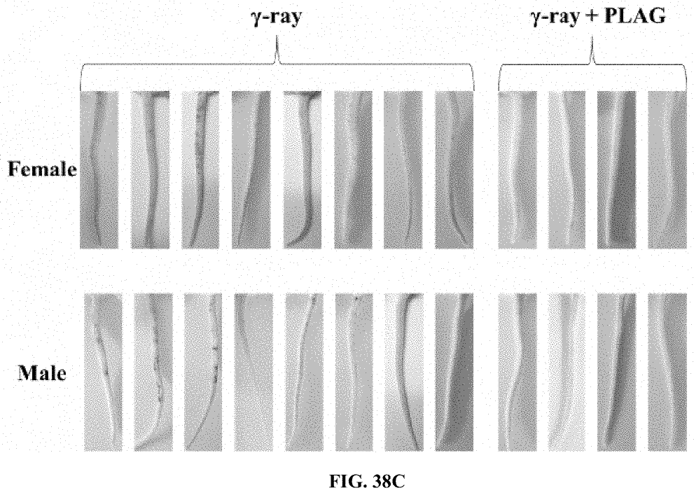

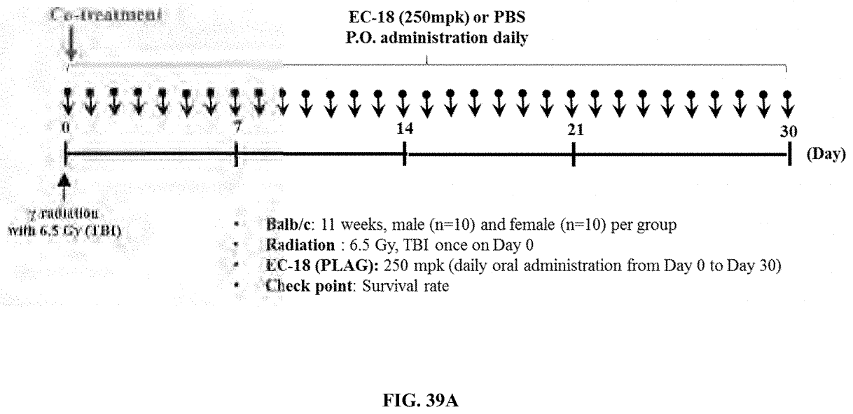

9 weeks Balb/c mice were divided into two groups: 1) radiated group (8Gy of TBI by γ-ray) and 2) PLAG (250 mg/kg of PLAG daily for 17 days) administrated group with radiation (8Gy of TBI) (FIG. 38A).

Results

While the radiated group showed severe erythema or burn, PLAG administered group with radiation showed significantly weak or no erythema on mouse foot and tail (FIG. 38B). Furthermore, for both female and mice male, PLAG administered group with radiation showed significantly weak or no erythema (FIG. 38C). From the clear improvement of skin erythema of mouse on foot and tail, PLAG is capable of protecting skin from tissue damage caused by lethal radiation.

### Example 39 PLAG Enhances the Survival Rate of Radiated Mice

Materials and Methods

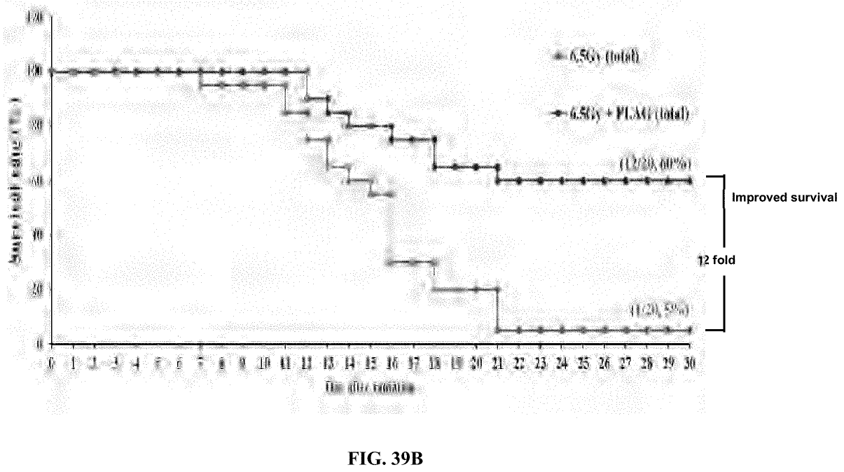

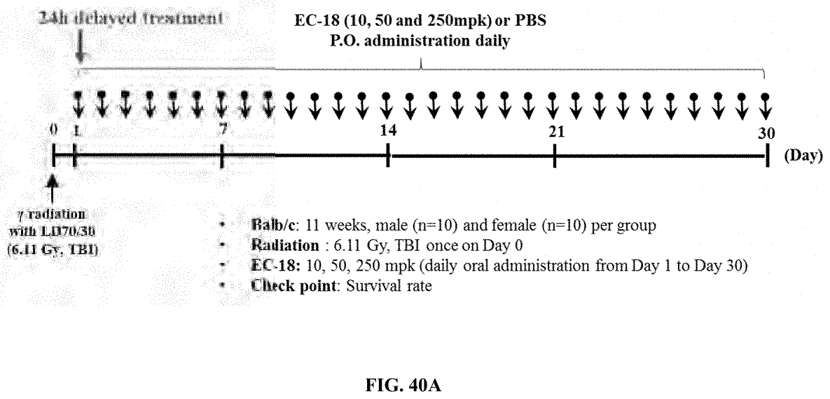

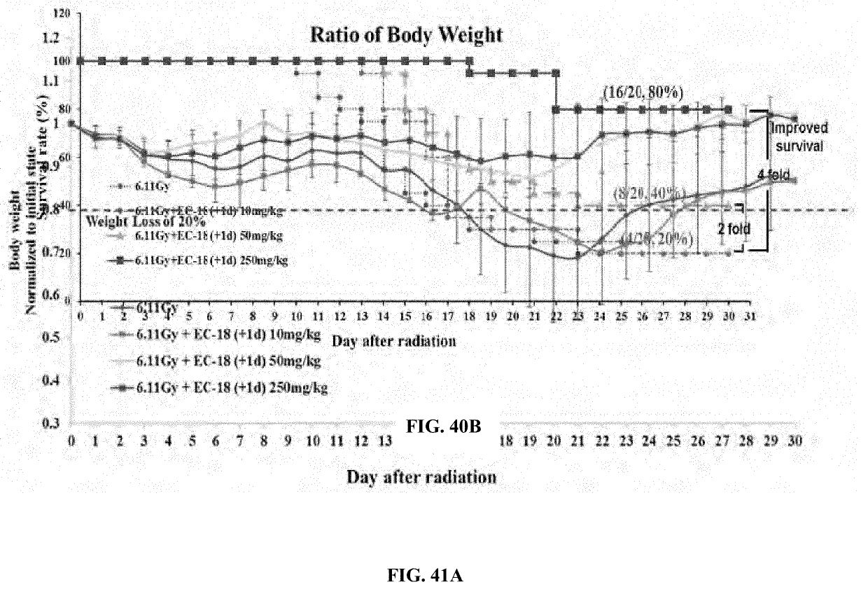

11 weeks Balb/c mice (10 males and 10 females) were divided into two groups: 1) radiated group (6.5Gy of TBI by γ-ray) and 2) PLAG (250 mg/kg of PLAG) administrated group with radiation (6.5Gy of TBI by γ-ray) (FIG. 39A). 6.5Gy of TBI was radiated on Day 0 and 250 mpk of PLAG was orally administrated from day 0 to day 30 daily. The survival rate of the mice was recorded daily until day 30.

Results

While the radiated group showed 5% survival rate 30 days after radiation, PLAG administered group showed a 60% survival rate 30 days after radiation, which is 12 times higher than the radiated group (FIG. 39B). The significantly high survival rate of PLAG administrated group with radiation support the function of PLAG to mitigate damage caused by lethal radiation, thereby improving the survival.

### Example 40 Dose Dependency of PLAG on the Survival Rate of Mice

Materials and Methods

11 weeks Balb/c mice (10 males and 10 females) were divided into four groups: 1) radiated group (6.11Gy of TBI by γ-ray), 2) PLAG (10 mg/kg of PLAG) administrated group with radiation (6.11Gy of TBI by γ-ray), 3) PLAG (50 mg/kg of PLAG) administrated group with radiation (6.11Gy of TBI by γ-ray), and 4) PLAG (250 mg/kg of PLAG) administrated group with radiation (6.11Gy of TBI by γ-ray) (FIG. 40A). 6.11Gy of TBI was radiated on Day 0 and 250 mpk of PLAG was orally administrated from day 1 to day 30 daily. The survival rate of the mice, was recorded daily until day 30.

Results

While radiated group and PLAG (10 mg/kg of PLAG) administrated group showed 5% of survival rate after radiation after 30 days of radiation, PLAG (50 mg/kg of PLAG) administrated group and PLAG (250 mg/kg of PLAG) administrated group showed 40% and 80% of survival rate after 30 days of radiation, respectively (FIG. 40B). The data indicate that 50mpk or higher of PLAG is highly promising to mitigate damage caused by lethal radiation, thereby improving the surviving rate. In particular, 250mpk of PLAG showed significantly efficacious to maintain a high survival rate of 80%.

### Example 41 Effects of PLAG on Body Weight of Mice

Materials and Methods

11 weeks Balb/c mice (10 males and 10 females) were divided into four groups: 1) radiated group (6.11Gy of TBI by γ-ray), 2) PLAG (10 mg/kg of PLAG) administrated group with radiation (6.11Gy of TBI by γ-ray), 3) PLAG (50 mg/kg of PLAG) administrated group with radiation (6.11Gy of TBI by γ-ray), and 4) PLAG (250 mg/kg of PLAG) administrated group with radiation (6.11Gy of TBI by γ-ray). 6.11Gy of TBI was radiated on Day 0, and 250 mpk of PLAG was orally administrated from day 1 to day 30 daily. Body weight of the mice was recorded daily until day 30.

Results

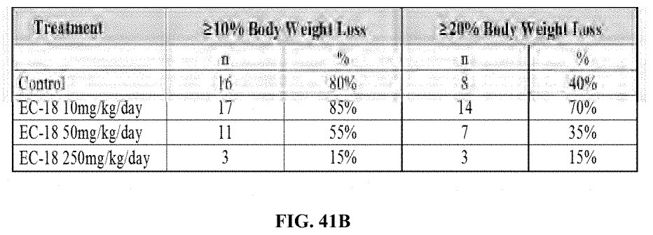

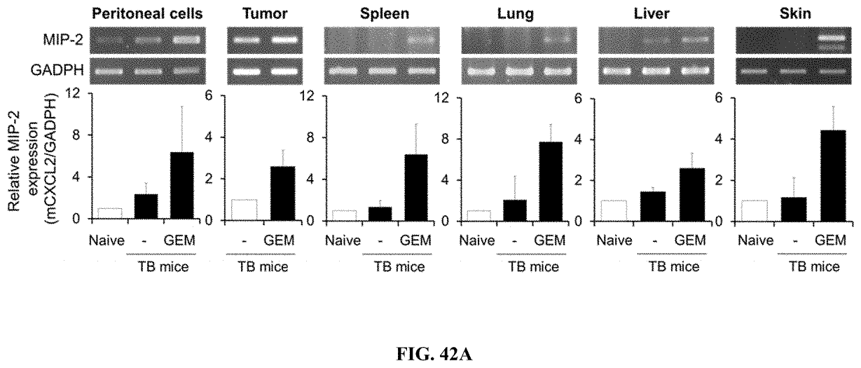

While radiated group and PLAG (10 mg/kg of PLAG) administrated group showed fluctuating and decreasing bodyweight after radiation, PLAG (50 mg/kg of PLAG) administrated group and PLAG (250 mg/kg of PLAG) administrated group showed less than about 10% of body weight loss (FIG. 41A). Control and 10mpk of PLAG showed a similar effect in terms of the number of mice whose body weight loss is more than 10% and 20% (FIG. 41B). 50mpk of PLAG and 250 mpk of PLAG contributed to the significantly less number of mice whose body weight loss is more than 10% and 20%. These data indicate that 50mpk or higher of PLAG is highly promising to mitigate damage caused by lethal radiation, thereby maintaining body weight. 50 mpk and 250 mpk of PLAG showed similar efficacy in maintaining body weight.

### Example 42 PLAG Modulates Gemcitabine-Induced CXCL2 and CXCL8

Materials and Methods

Animals

Male BALB/c mice (6-8 weeks of age, 20-22 g) were purchased from Koatech Corporation (South Korea) and maintained in a specific pathogen-free facility under consistent temperature and 12-h light/dark cycles. All experimental procedures were approved by the Institutional Animal Care and Use Committee of the Korea Research Institute of Bioscience and Biotechnology (South Korea) and performed in compliance with the National Institutes of Health Guidelines for the care and use of laboratory animals and Korean national laws for animal welfare.

Gemcitabine-Induced Neutropenia Mice Model

The figure shows a schematic illustration of the protocols. Male BALB/c mice were randomly divided into 3 groups; normal control group (n=5), gemcitabine only group (n=5) and gemcitabine with PLAG group (n=5). The mice were intraperitoneally (i.p.) injected with 50 mg/kg gemcitabine to induce neutropenia. PLAG was diluted with phosphate-buffered saline (PBS) and then orally administrated at a dose of 50 or 250 mg/kg/day. The normal control group was administered PBS only during the experiment. The whole blood was collected from the orbital sinuses using capillary tubes (Kimble Chase Life Science and Research Products LLC, FL, USA) and collection tubes containing K3E-K3EDTA (Greiner Bio-One International, Germany). To obtain peritoneal cells, 5 ml of cold PBS was injected to the left side of the peritoneal wall using a 5 mL syringe, and the fluid was aspirated from the peritoneum. The collected cells were counted by complete blood count (CBC) analysis using Mindray BC-5300 auto-hematology analyzer (Shenzhen Mindray Biomedical Electronics, China). To establish a 4T1 tumor-bearing mice model, the murine 4T1 mammary carcinoma cells (1×10⁵) were subcutaneously injected on the right side of the abdomen. On the 10th day after tumor injection, the mice were intraperitoneally (i.p.) injected with 50 mg/kg gemcitabine, and the next day the whole blood was collected from the orbital sinuses as mentioned above before sacrificing the animals to obtain different organs of the body for RT-PCR.

RT-PCR and Real-Time PCR

Total RNA was extracted using the Total RNA Extraction Solution (Favorgen, Taiwan), according to the manufacturer's instructions. This RNA was used in reverse transcription reactions with oligo-dT primers and M-MLV RT reagents (Promega, Madison, Wis., USA), according to the manufacturer's instructions. For RT-PCR, the synthesized cDNA was mixed with 2×PCR Master Mix (Solgent, Daejeon, Republic of Korea) and 10 pmol specific PCR primer pair following the manufacturer's protocol. The primers were synthesized from Macrogen (Seoul, Republic of Korea; see Table 2 for primer sequences). Amplified products were separated on 1% agarose gels, stained with ethidium bromide, and photographed under UV illumination using a GelDoc (Bio-Rad Laboratories, Hercules, Calif., USA).

An SYBR Green kit (Applied Biosystems, Foster City, Calif., USA) was used for real-time PCR (qPCR) analysis of cDNA according to the manufacturer's instructions. Thermal cycling conditions were as follows: initial denaturation at 95° C. for 15 minutes, followed by 40 cycles of 95° C. for 30 seconds, 60° C. for 30 seconds, and 72° C. for 30 seconds. A melting step was performed by raising the temperature from 72° C. to 95° C. after the last cycle. Thermal cycling was conducted on a ViiA 7 Real-Time PCR System machine (Applied Biosystems). The target gene expression levels are shown as a ratio in comparison with GAPDH expression in the same sample by calculation of cycle threshold (Ct) value. The relative expression levels of target genes were calculated by the 2^{-ΔΔCT} relative quantification method. GAPDH was used as a control.

Enzyme-Linked Immunosorbent Assay (ELISA)

The concentration of MIP-2 was measured using ELISA kits for MIP-2 (R&D Systems, Minneapolis, Minn., USA according to the manufacturers' instructions. Cytokine levels were estimated by interpolation from a standard curve generated using an ELISA reader (Molecular Devices) at 450 nm.

Results

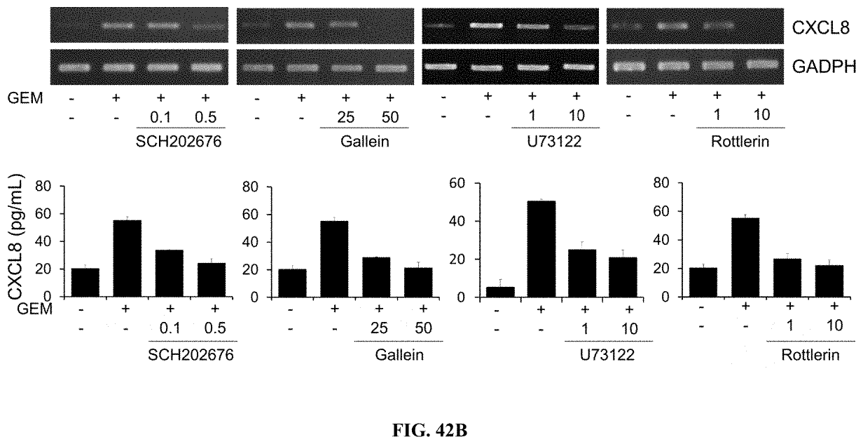

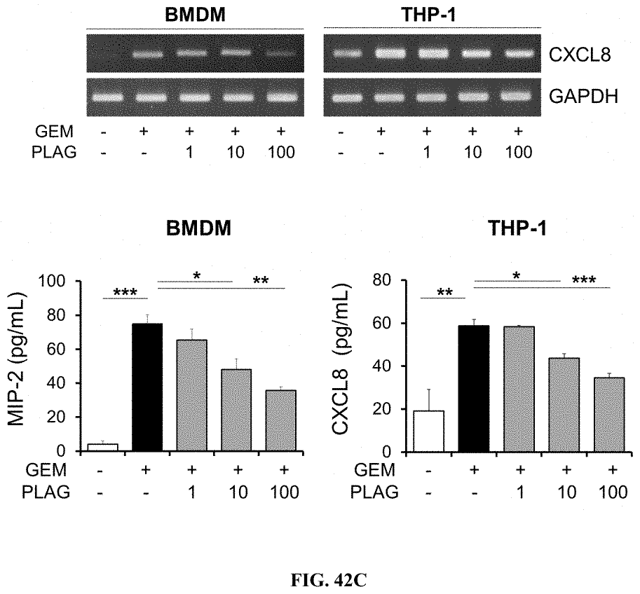

Chemotherapeutic agents generally induce tissue damage, which also subsequently triggers chemokine expression. From all tissues including peritoneal cells, implanted tumor, spleen, lung, liver and skin, gemcitabine increases the mRNA expression of neutrophil attracting chemokine MIP-2, which is a small cytokine that induces mobilization of neutrophils by interacting with its receptor CXCR2 (FIG. 42A). These results indicated that gemcitabine may induce extravasation of circulating neutrophils and infiltration into the peritoneum and peripheral tissues through the interaction of chemokines with its receptors.

Gemcitabine also induces CXCL8 in the human monocyte, THP-1. Inducement of CXCL8 is initiated from the recognition of gemcitabine by gemcitabine receptor (adenosine receptor) and its sequential cascade is delivered by G-protein coupled receptor (GPCR), phospholipase C (PLC), and protein kinases C (PKC). Using antagonists for GPCR, PLC, and PKC, the reduction of CXC8 expression was confirmed with a dose-dependent fashion (FIG. 42B). These observations indicate that the GPCR/G protein/PLC/PKC signaling pathway is involved in gemcitabine-induced CXCL8 production in macrophages.

Neutrophil migration is initiated by chemokine and neutrophil moves toward chemokine gradients. Gemcitabine induces chemokine CXCL8 in the primary cell bone marrow-derived macrophage (BMDM) and monocyte cell line THP-1. The levels of secreted gemcitabine-induced chemokines MIP2 and CXCL8 were effectively reduced by PLAG with a dose-dependent manner in the transcriptional level and secreted protein level (FIG. 42C).

### Example 43 PLAG Modulates Gemcitabine-Induced ROS Production in BMDMs and THP-1 Cells

Materials and Methods

Intracellular ROS Measurement

A total of 1×10⁶ BMDMs and THP-1 were seeded, cultured, and subsequently exposed to various concentrations of PLAG with gemcitabine (10 μg/mL) for 3 h. The cells were then incubated with the ROS-sensitive probe CM-H2DCFDA (Invitrogen™) for 30 min at 37° C. in the dark. After incubation, the cells were washed 3 times with PBS and immediately analyzed using FACS verse (BD biosciences) with an excitation/emission peak at 495/527 nm. A total of 10,000 cells were counted in each determination, and results presented are means±S.E. of three independent experiments. Intracellular ROS production was also measured with a confocal laser scanning microscope (Zeiss LSM 800, Oberkochen, Germany). After incubating CM-H₂DCFDA as above, the cells were fixed with 4% paraformaldehyde for 30 min and washed 3 times with PBS before photographing. The excitation and emission wavelengths were identical as described above, and a minimum of 5 random fields was captured for each culture.

Statistical Analysis.

All experiments were performed in triplicate and the results were expressed as the mean±standard deviation (SD). Statistical analysis was performed using a Student's unpaired t-test and p values <0.05 were considered statistically significant.

Reverse Transcription Polymerase Chain Reaction (RT-PCR)

Total RNAs from cells were isolated using TRIzol® reagent (Invitrogen, CA, USA) according to the manufacturer's instructions. RT-PCR was performed using PCR reagent (Bioassay, Daejeon, South Korea). Complementary DNA (cDNA) was synthesized from total RNA using an RT kit (Bioassay), followed by conventional PCR. The primers used in this study are as follows: human CXCL8, 5′-AGGGTTGCCAGATGCAATAC-3′ (SEQ ID NO. 11) and 5′-GTGGATCCTGGCTAGCAGAC-3′ (SEQ ID NO. 12); mouse MIP-2, 5′-AGTGAACTGCGCTGTCAATG-3′ (SEQ ID NO. 1) and 5′-CTTTGGTTCTTCCGTTGAGG-3′ (SEQ ID NO. 2); GAPDH, 5′-CCATCACCATCTTCCAGGAG-3′ (SEQ ID NO. 7) and 5′-ACAGTCTTCTGGGTGGCAGT-3′ (SEQ ID NO. 8).

Membrane Fractionation and Immunoblotting

Total cells were lysed on ice for 30 minutes in RIPA buffer composed of 50 mM Tris-HCl (pH 7.4), 150 mM NaCl, 2 mM EDTA, 1% NP-40, 0.5% sodium deoxycholate, 0.1% SDS, 1 mM phenylmethylsulfonyl fluoride (PMSF), 10 mM sodium fluoride, 2 mM sodium pyrophosphate, 10 mM β-glycerophosphate, 10 mM sodium orthovanadate. Membrane-and-cytoplasmic protein fractions of cultured cells were obtained with Mem-PER Plus Membrane Protein Extraction Kit (Thermo Scientific, MA, USA) according to the manufacturer's instructions. The lysates were centrifuged at 13,000 rpm for 20 minutes at 4° C. and protein concentrations were determined using the Bradford assay (Bio-Rad Laboratories). Denatured samples were mixed with a 5×SDS-PAGE loading buffer and heated to 100° C. for 15 min. The samples were separated on the 10% of SDS-PAGE gel and transferred to polyvinylidene difluoride (PVDF) membranes (Merck Millipore Corporation, MA, USA). Membranes were blocked with 5% non-fat milk in PBS (10 mM Tris-HCl, pH7.5, 150 mM NaCl) for 1 h and probed with primary antibodies against ERK1/2, phospho-ERK1/2, P38, phospho-P38, SAPK/JNK, phospho-SAPK/JNK, Na, K-ATPase, α-Tubulin, and β-actin from Cell Signaling Technology (MA, USA), Rac1 from Merck Millipore Corporation), p47phox and phospho-p47phox from Invitrogen, adenosine receptor A1 from Abcam, β arrestin-1 from Santa Cruz Technology for overnight at 4° C. The blots were washed and incubated with appropriate secondary antibodies and visualized using PierceECL Western Blotting Substrate (Thermo Scientific).

Results

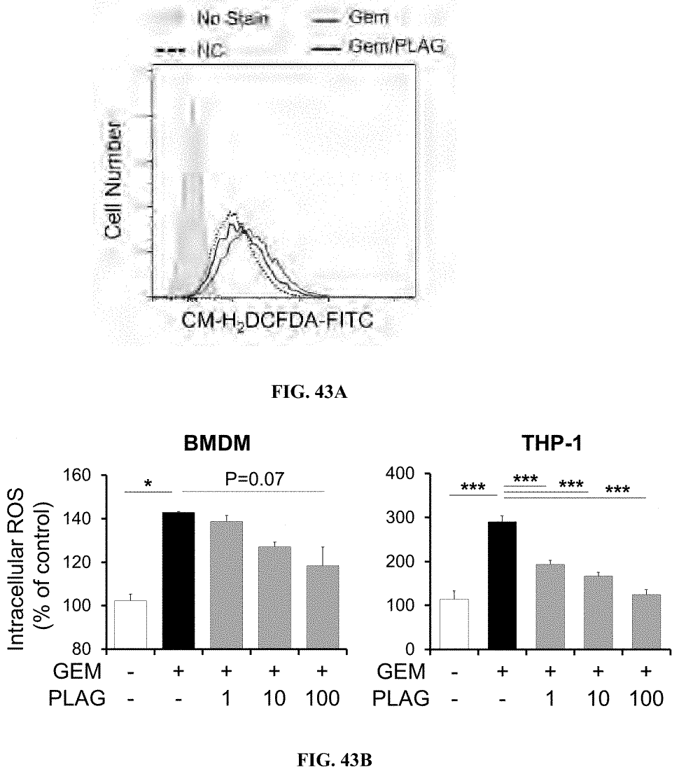

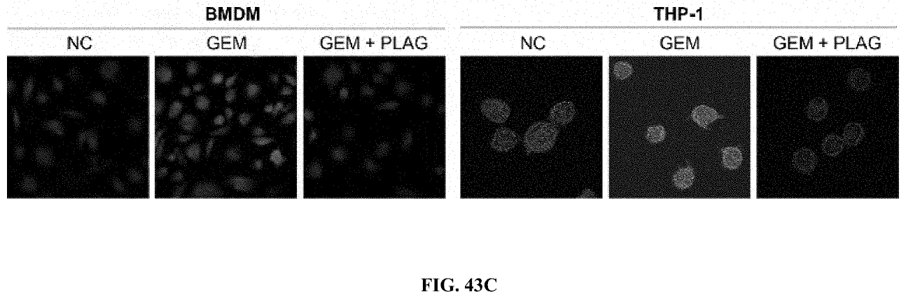

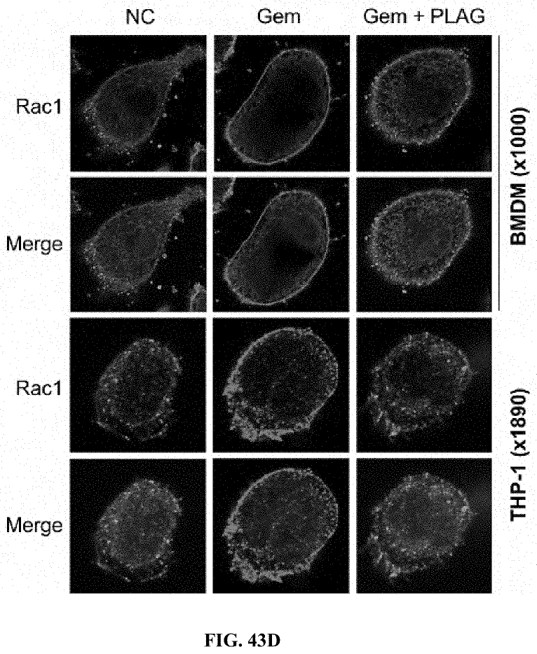

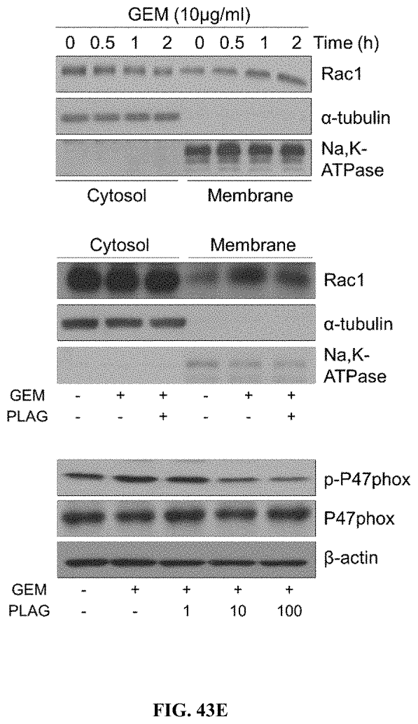

Gemcitabine-induced reactive oxygen species (ROS) production was fully examined by flow cytometry (FIG. 43A) and confocal microscopy (FIG. 43C). Flow cytometry results show that PLAG co-treated cells showed a smaller mean of chloromethyl derivative of 2′, 7′-dichlorodihydrofluorescein diacetate (CM-H2DCFDA) than Gemcitabine treated cells did. This indicates that ROS production induced by gemcitabine was reduced by PLAG because CM-H2DCFDA is an indicator of reactive oxygen species (ROS) in cells. Further, ROS production induced in the gemcitabine treated cells was well visualized with the confocal image. PLAG effectively reduced gemcitabine-induced intracellular ROS production with a dose-dependent manner (FIG. 43B). These data suggest that PLAG accelerates the initiation of intracellular GPCR trafficking and decreases the duration of intracellular GPCR trafficking with endosome, which leads to reduce gemcitabine-induced intracellular ROS.

The activity of ROS-producing enzyme, NOXs, was examined in the gemcitabine treated cells. Rac1. p47phox is major component of NOXs. For the production of ROS, enzymes like as Rac1 and p47 was clustered toward the membrane. Cytosolic Rac1 gradually reduced, while membrane Rac1 is gradually increased with time-dependent manner. Polarized Rac1 into membrane was observed in the gemcitabine treated cells. Polarized Rac1 returns to cytosol and level of Rac1 into membrane was decreased in the PLAG treated cells. Phosphorylation of p47 has slightly increased in the gemcitabine treated cells and gradually reduced in the PLAG treated cell with dose-dependent.

PLAG remarkably prevented gemcitabine-induced Rac1 membrane translocation in BMDMs and THP-1 cells (FIG. 43D). The membrane and cytosolic fractions isolated from gemcitabine- and/or PLAG-stimulated THP-1 cells confirmed that gemcitabine increased membrane translocation of Rac1 in a time-dependent manner (FIG. 43E, top), and PLAG significantly inhibited translocation of Rac1 from the cytosol to the membrane (FIG. 43E, middle). The cytosolic component of p47phox migrates instantly to the membrane upon stimulation and assembles with the membrane components to form the active enzyme. This process is tightly regulated by the phosphorylation of p47phox. Next, the effect of PLAG on gemcitabine-induced phosphorylation of p47phox was examined, and as expected, PLAG effectively inhibited p47phox phosphorylation in a dose-dependent manner (FIG. 43E, bottom). These data indicate that PLAG decreases gemcitabine-generated ROS production by inhibiting the activation of NOX2 via inhibition of Rac1 membrane translocation and p47phox phosphorylation.

### Example 44 PLAG Modulates Gemcitabine-Induced Phosphorylation of ROS Dependent Signal Molecules

Materials and Methods

Membrane Fractionation and Immunoblotting

Total cells were lysed on ice for 30 minutes in RIPA buffer composed of 50 mM Tris-HCl (pH 7.4), 150 mM NaCl, 2 mM EDTA, 1% NP-40, 0.5% sodium deoxycholate, 0.1% SDS, 1 mM phenylmethylsulfonyl fluoride (PMSF), 10 mM sodium fluoride, 2 mM sodium pyrophosphate, 10 mM β-glycerophosphate, 10 mM sodium orthovanadate. Membrane-and-cytoplasmic protein fractions of cultured cells were obtained with Mem-PER Plus Membrane Protein Extraction Kit (Thermo Scientific, MA, USA) according to the manufacturer's instructions. The lysates were centrifuged at 13,000 rpm for 20 minutes at 4° C. and protein concentrations were determined using the Bradford assay (Bio-Rad Laboratories). Denatured samples were mixed with a 5×SDS-PAGE loading buffer and heated to 100° C. for 15 min. The samples were separated on the 10% of SDS-PAGE gel and transferred to polyvinylidene difluoride (PVDF) membranes (Merck Millipore Corporation, MA, USA). Membranes were blocked with 5% non-fat milk in PBS (10 mM Tris-HCl, pH7.5, 150 mM NaCl) for 1 h and probed with primary antibodies against ERK1/2, phospho-ERK1/2, P38, phospho-P38, SAPK/JNK, phospho-SAPK/JNK, Na, K-ATPase, α-Tubulin, and β-actin from Cell Signaling Technology (MA, USA), Rac1 from Merck Millipore Corporation), p47phox and phospho-p47phox from Invitrogen, adenosine receptor A1 from Abcam, β arrestin-1 from Santa Cruz Technology for overnight at 4° C. The blots were washed and incubated with appropriate secondary antibodies and visualized using Pierce ECL Western Blotting Substrate (Thermo Scientific).

Results

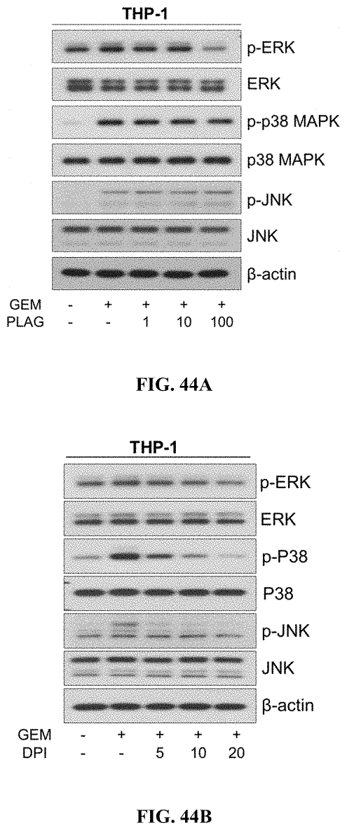

Gemcitabine upregulated phosphorylation of members of the ROS dependent mitogen-activated protein kinase (MAPK) superfamily, including ERK, p38 MAPK, and JNK, in a time-dependent manner. The effect of PLAG on gemcitabine-induced phosphorylation of ERK, p38 MAPK and JNK was assessed. PLAG dose-dependently decreased the phosphorylation of ERK and p38 MAPK but did not for JNK (FIG. 44A). DPI, an inhibitor of NADPH oxidase, also dose-dependently decreased the gemcitabine-induced phosphorylation of ERK, p38 MAPK and JNK (FIG. 44B). These results indicate that PLAG modulates gemcitabine-induced phosphorylation of ROS-dependent signal molecules.

### Example 45 PLAG Modulates Gemcitabine-Induced Neutrophil Extravasation

Materials and Methods

Flow Cytometry

PLAG inhibits gemcitabine-induced neutrophil extravasation into the peritoneum by down-regulating the expression of adhesion molecules in normal BALB/c mice. Male BALB/c mice of 8-10 weeks of age were orally administrated with 50 or 250 mg/kg of PLAG and then were intraperitoneally injected with 50 mg/kg gemcitabine. After 24 h, blood samples were collected by retro-orbital bleeding, and the number of blood neutrophils was determined by CBC analysis. Each group contains five mice. The population of blood neutrophils was analyzed by flow cytometry. Red cell-lysed whole blood was stained with FITC-conjugated anti-Ly6G and PE-Cy7-conjugated anti-CD11b antibodies to determine the circulating neutrophil population. Ly6G+/CD11b+ cells were further stained with APC-conjugated anti-L-selectin and APC-conjugated anti-LFA-1 antibodies and were analyzed by flow cytometry to determine the expression of adhesion molecules.

Statistical Analysis

All experiments were performed in triplicate, and the results were expressed as the mean±standard deviation (SD). For comparison of the statistical differences of more than two groups, one-way ANOVA test was used and p values <0.05 were considered statistically significant.

Gemcitabine-Induced Neutropenia Mice Model

The mice were intraperitoneally (i.p.) injected with 50 mg/kg gemcitabine to induce neutropenia. PLAG was diluted with phosphate-buffered saline (PBS) and then orally administrated at a dose of 50 or 250 mg/kg/day. The normal control group was administered PBS only during the experiment. At 15 h after gemcitabine treatment, the whole blood was collected from the orbital sinuses using capillary tubes (Kimble Chase Life Science and Research Products LLC, FL, USA) and collection tubes containing K3E-K3EDTA (Greiner Bio-One International, Kremsmünster, Austria). To obtain peritoneal cells, 5 ml of cold PBS was injected to the left side of the peritoneal wall using a 5 mL syringe, and the fluid was aspirated from the peritoneum. The collected cells were counted by complete blood count (CBC) analysis using Mindray BC-5300 auto-hematology analyzer (Shenzhen Mindray Biomedical Electronics, Guangdong Sheng, China).

To establish a 4T1 tumor-bearing mice model, the murine 4T1 mammary carcinoma cells (1×105) were subcutaneously injected on the right side of the abdomen. On the 10th day after tumor injection, the mice were intraperitoneally (i.p.) injected with 50 mg/kg gemcitabine, and the next day the whole blood was collected from the orbital sinuses as mentioned above before sacrificing the animals to obtain different organs of the body.

Results

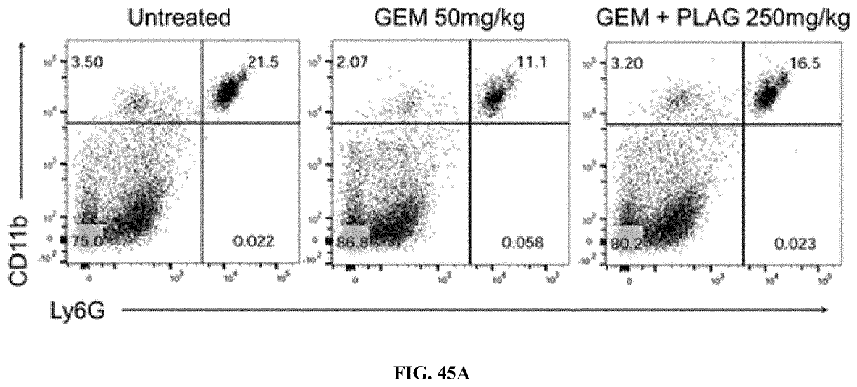

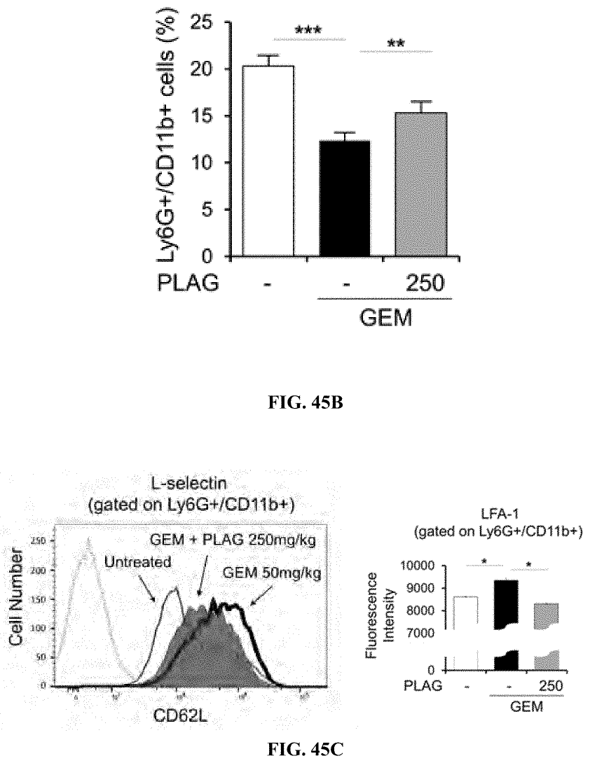

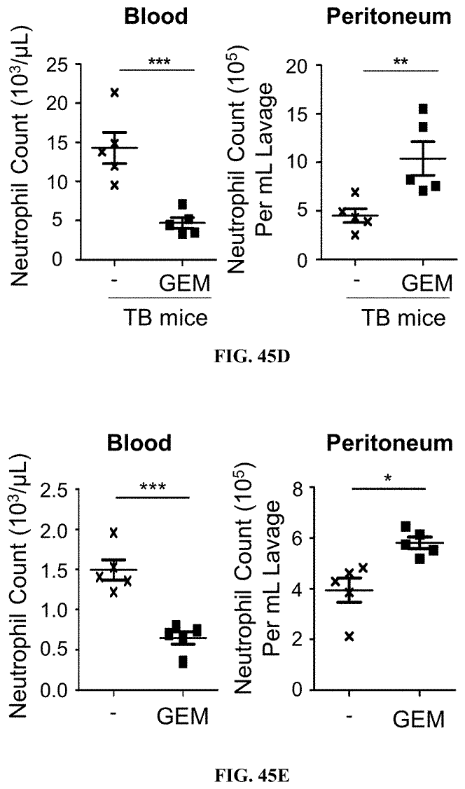

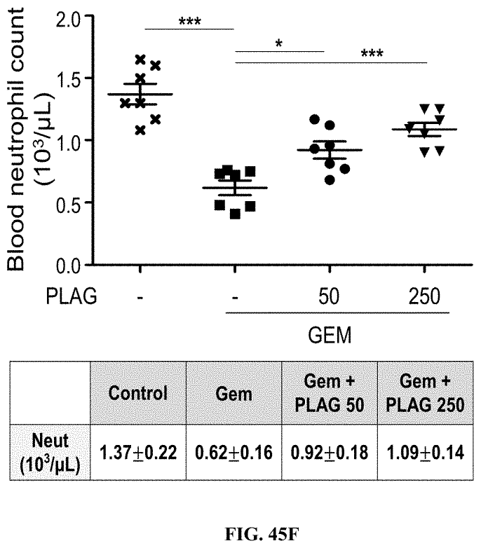

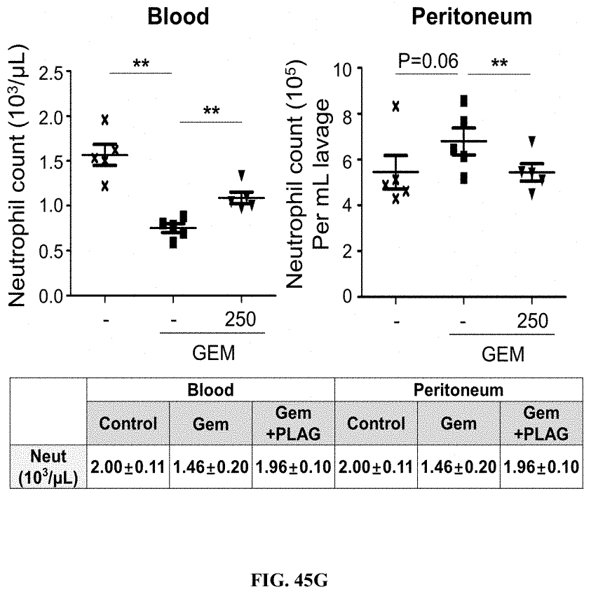

Since the peritoneal neutrophil counts increased following gemcitabine treatment, while the blood neutrophil counts decreased, active neutrophil transmigration sometimes might be a major cause of neutropenia in the gemcitabine treated mice. It was next investigated whether gemcitabine provokes neutrophil extravasation by measuring the population of neutrophil markers LY6G+/CD11b+. First, it was confirmed that PLAG maintained the population of Gr-1-positive (Gr-1+) and CD11b-positive (CD11b+) neutrophils in the blood, which was decreased by gemcitabine (FIGS. 45A and 45B). Cell surface expression of the adhesion molecules, L-selectin and LFA-1, which mediate extravasation of Gr-1+/CD11b+ cells, was examined. As a result, it was observed that PLAG effectively inhibited gemcitabine-induced cell surface expression of these adhesion molecules (FIG. 45C). These observations suggest that PLAG has a significant effect on preventing gemcitabine-induced neutrophil migration by down-regulating the surface expression of adhesion molecules.

A mouse model of breast cancer by injecting BALB/c mice with the murine 4T1 mammary carcinoma cells subcutaneously to the abdomen was established to study gemcitabine-induced changes in the kinetics of neutrophils. A single intraperitoneal (i.p.) gemcitabine (50 mg/kg) was administered to the mice after 10 days of the injection. The mice were sacrificed and analyzed one day after the administration. The administered gemcitabine-induced the migration of circulating neutrophils into the peritoneal cavity (FIG. 45D).

Next, 50 mg/kg of gemcitabine was injected to normal BALB/c mice to see whether the same phenomenon happens in non-tumor-bearing mice. A single intraperitoneal (i.p.) gemcitabine (50 mg/kg) was administered to Balb/c mice. The mice were sacrificed and analyzed 15 hours after the administration. There was a significant reduction of circulating neutrophils at 15 hours after gemcitabine treatment, while an increase of neutrophils in the peritoneal cavity (FIG. 45E). Therefore, gemcitabine induces the depletion of circulating neutrophils with or without cancer.

Further experiments using normal BALB/c mice were performed. To investigate whether PLAG affects gemcitabine-induced neutropenia, PLAG (50 and 250 mg/kg) was orally administrated to the mice just before gemcitabine treatment (i.p. injection; 50 mg/kg). After 15 hours, gemcitabine-induced a sharp decrease of circulating neutrophil counts compared to the untreated control, and administration of PLAG restored circulating neutrophils to an almost normal range in a dose-dependent manner (FIG. 45F). The number of neutrophils in the peritoneal cavity was examined, and it was observed that PLAG effectively decreases neutrophil counts in the peritoneum that were elevated 15 hours after gemcitabine treatment (FIG. 45G). Increased circulating neutrophil and decreased peritoneal neutrophil by PLAG treatment indicates that PLAG effectively inhibits neutrophil transmigration.

### Example 46 PLAG Modulates 5-FU-Induced Utneutropenia and Reduction of Monocyte in Mice

Materials and Methods

Animals

Specific-pathogen-free male and female BALB/c mice (7 weeks of age) were obtained from Koatech Co. (Pyongtaek, Republic of Korea). Upon receipt, the mice were housed, 5 per cage, in a specific pathogen-free facility, and acclimatized for 1 week under conditions of consistent temperature and normal light cycles. All the animals were fed a standard mouse diet with water allowed ad libitum. All experimental procedures were approved by the Institutional Animal Care and Use Committee of the Korea Research Institute of Bioscience and Biotechnology and were performed in compliance with the National Institutes of Health guidelines for the care and use of laboratory animals and Korean national laws for animal welfare.

Establishment of 5-FU-Induced Neutropenia in Mice and Investigation of the Influence of Administration of EC-18 on the Kinetics of Blood Neutrophils in 5-FU-Treated Mice

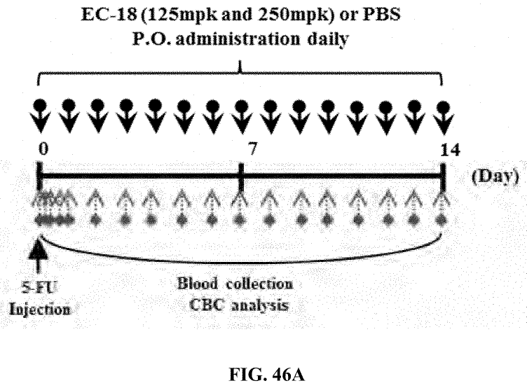

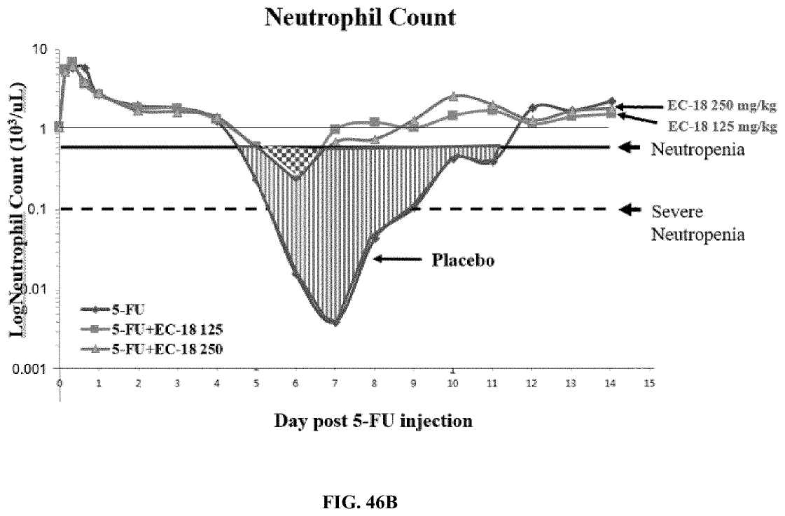

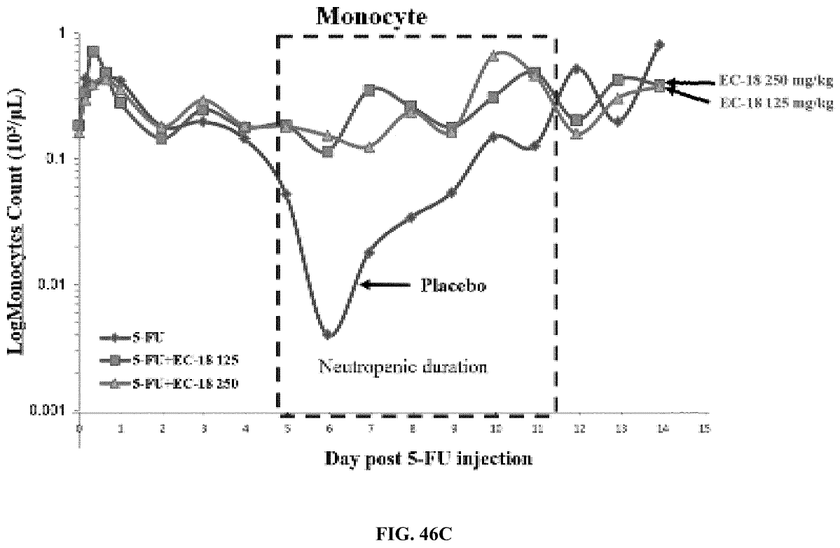

Male BALB/c mice (8 weeks of age) were randomly divided into 3 cohorts; control cohort (n=5), EC-18 125 mg/kg-treated cohort (n=5) and EC-18 250 mg/kg (n=5). The mice were intraperitoneally (i.p.) injected once with 100 mg/kg 5-FU to induce neutropenia, as shown in the experimental scheme (FIG. 46A). EC-18 was suspended in phosphate-buffered saline (PBS) and orally administrated at a dose of 125 or 250 mg/kg once a day, starting on the same day of 5-FU injection. The control cohort was administered PBS only during the experiment. The whole blood was collected from the orbital sinuses using EDTA-free capillary tubes (Kimble Chase Life Science and Research Products LLC, FL, USA) and collection tubes containing K3E-K3EDTA (Greiner Bio-One International, Kremsmünster, Austria). The blood cells were counted and classified by complete blood count (CBC) analysis using Mindray BC-5000 auto-hematology analyzer (Shenzhen Mindray Biomedical Electronics, Guangdong Sheng, China).

Statistical Analyses

The results were expressed as the mean±standard deviation (SD). Statistical analysis was performed using a Student's paired t-test and p values <0.05 were considered statistically significant. A paired Log-rank (Mantel-Cox) test was used to compare the duration of neutropenia and time to recovery from neutropenia between control and EC-18-treated cohorts.

Results

A single injection of 5-FU 100 mg/kg reduced the ANC in control, EC-18 125 and EC-18 250 mg/kg-treated cohort from pre-injection values to <500 cells/μL by 5.2±0.45, 5.8±0.45 and 5.8±0.45 days, respectively (FIG. 46B and Table 2). The administration of EC-18 in 5-FU-injected mice resulted in a significant reduction in the duration of neutropenia and the time to recovery of ANC>1000 cells/μL. EC-18 125 or 250 mg/kg significantly reduced the length of neutropenia from 7.4±1.14 days to 2.6±0.55 and 3.0±0.71 days, respectively (FIG. 46B and Table 2). Moreover, the ANC of all individuals in the control cohort fell to a severely neutropenic range (ANC<100 cells/μL), while only 20% of individuals in both EC-18 125 and 250 mg/kg-treated cohorts experienced severe neutropenia. EC-18 also reduced the duration of severe neutropenia from 5.2±1.48 days to 2 days (Table 3). EC-18 125 or 250 mg/kg administration significantly increased the mean nadir after 5-FU injection from 2.0±4.47 cells/μL to 236±4.47 or 158±11.32 cells/μL, respectively (Table 4). The time of recovery to an ANC≥500 or 1000 cells/μL was significantly reduced in EC-18 125 and 250 mg/kg-treated cohorts.

TABLE 2
Mean First Day of Neutropenia (ANC <500 cells/μL),
and Mean Duration of Neutropenia in Control, EC-18 125mpk
and EC-18 250mpk-treated mice injected with 5-FU 100mpk

| | Mean the | Mean Duration |
|---|---|---|
| | First Day | of Neutropenia |
| | Neutropenia | in Days |
| Treatment | (±SE, range) | (±SE, range) |
| Control | 5.2 ± 0.45 (5-6) | 7.4 ± 1.14 (5-13) |
| EC-18 125 mg/kg | 5.8 ± 0.45 (5-6) | 2.6 ± 0.55 (5-8) |
| EC-18 250 mg/kg | 5.8 ± 0.45 (5-6) | 3.0 ± 0.71 (5-8) |
| Two-sided P value | 0.07 | 0.002 |
| (Control vs. EC-18 | | |
| 125 mg/kg) | | |
| Two-sided P value | 0.07 | <0.001 |
| (Control vs. EC-18 | | |
| 250 mg/kg) | | |

TABLE 3
Number of Individuals of Severe Neutropenia (ANC <100 cells/μL),
and Mean Duration of Severe Neutropenia in Control, EC-18 125mpk
and EC-18 250mpk-treated mice injected with 5-FU 100mpk

| | | Mean Duration |
|---|---|---|
| | | of Severe |
| | Number of | Neutropenia |
| | Individuals of | in Days |
| Treatment | Severe Neutropenia | (±SE, range) |
| Control | 5/5 | 5.2 ± 1.48 (5-11) |
| EC-18 125 mg/kg | 1/5 | 2 (6-7) |
| EC-18 250 mg/kg | 1/5 | 2 (6-7) |

TABLE 4
Mean Nadir and Recovery from Neutropenia in Control, EC-18
125 and EC-18 250-Treated mice injected with 5-FU 100 mg/kg

| | | Mean Number | Mean Number |
|---|---|---|---|
| | | of Days to | of Days to |
| | | Recovery - | Recovery - |
| | Nadir of ANC | ANC ≥ 500/μL | ANC ≥ 1000/μL |
| Treatment | (cells/μL) | (±SE, range) | (±SE, range) |
| Control | 2.0 ± 4.47 | 11.6 ± 1.14 (10-13) | 11.8 ± 1.09 (10-13) |
| EC-18 125 mg/kg | 236 ± 121.57 | 7.4 ± 0.55 (7-8) | 8 ± 1.22 (7-10) |
| EC-18 250 mg/kg | 158 ± 11.32 | 7.6 ± 0.55 (7-8) | 8.8 ± 1.09 (7-10) |
| Two-sided | 0.012 | 0.001 | 0.004 |
| P value | | | |
| (Control vs. | | | |
| EC-18 125 mg/kg) | | | |
| Two-sided | 0.038 | 0.003 | 0.023 |
| P value | | | |
| (Control vs. | | | |
| EC-18 250 mg/kg) | | | |

Further, a single treatment of 5-FU induced the reduction of blood monocytes, similar to the pattern of the decrease of neutrophil counts (FIG. 46C). The number of blood monocytes decreased during a period corresponding to neutropenic duration. The administration of EC-18 125 or 250mpk in 5-FU-injected mice remarkably mitigated the reduction of blood monocytes.

### Example 47 PLAG Modulates Chemotherapy-Induced Neutropenia in Human Patients

Materials and Methods

Patients

From January 2014 to September 2014, 16 patients with histologically or cytologically confirmed unresectable pancreatic cancer were enrolled in this study. Eligible patients had 1) locally advanced or metastatic cancer; 2) an age of ≥18 years; 3) an Eastern Cooperative Oncology Group (ECOG) performance status of ≤1; 4) adequate bone marrow function (absolute neutrophil count (ANC)≥1,500/mm³, platelet count ≥105/mm³); 5) normal renal (creatinine clearance ≥50 mL/min) and hepatic function (alanine aminotransferase and total bilirubin ≥2 times the upper limit of normal). Historical controls were also recruited from Asan Medical Center from March 2012 to December 2013. The eligibility criteria for the control group were the same as those for cases who intake PLAG during gemcitabine-based chemotherapy. The control group (n=32) was matched to the PLAG group (n=16) based on age, performance status, chemotherapy cycle, comorbidity, and disease extent. This study was approved by a hospital institutional review board.

Study Design and Treatment Protocol

All patients received gemcitabine 1,000 mg/m² on days 1, 8, and 15 of each 4-week schedule and daily erlotinib at 100 mg orally. In the PLAG group, PLAG 500 mg was orally administered twice daily from the start of the chemotherapy to the completion. Hematology and serum chemistry analyses were performed at screening baseline, then weekly until the end of the study. Febrile neutropenia (FN) was defined as an ANC of less than 1,000/mm³ and an oral temperature of more than 38° C. on the same day or the following day after chemotherapy. If on the day of chemotherapy administration, a patient's ANC was reduced to 500-1,000/mm³ or if the absolute platelet count was reduced to 50,000-100,000/mm³, the gemcitabine dose was reduced by 75%. Gemcitabine was omitted for 1 week if the neutrophil count was lower than 500/mm³, or the absolute platelet count was lower than 50,000/mm³. Chemotherapy was discontinued if disease progression was observed in a follow-up CT scan, which was performed within 2 or 3 months after the initiation of chemotherapy. Erlotinib dose was interrupted in patients within tolerable rash and was reduced or discontinued if symptoms persisted for 10-14 days. Erlotinib dose was reduced for grade 2 diarrhea persisting for 48-72 h and for grade 3 diarrhea following resolution to grade 1; erlotinib was permanently discontinued for grade 4 diarrhea. Treatment continued until disease progression unacceptable toxicity, withdrawal of patient's consent or physician's decision. Safety was evaluated throughout the entire study. Toxicity was graded based on the NCI Common Terminology Criteria for Adverse Events (CTCAE) version 3.0.

Statistical Analysis

The primary endpoint was neutropenia, and the secondary endpoint was a safety profile. All analyses were performed using SPSS version 17.0 (SPSS Inc., Chicago, Ill., USA). Descriptive statistics were used to evaluate demographics, and safety data continuous variables were compared using the Mann-Whitney U test, paired t-test, and independent T-test. A P value of <0.05 was considered statistically significant.

Results

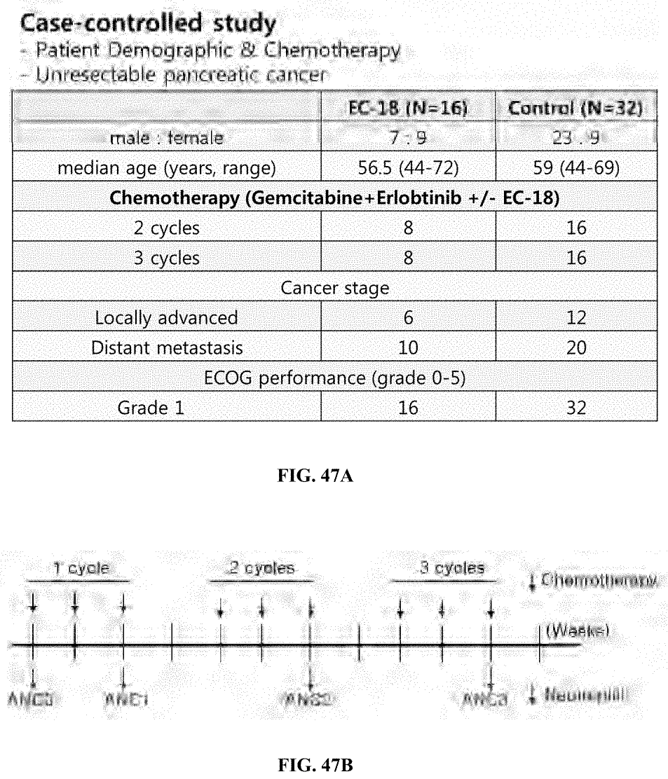

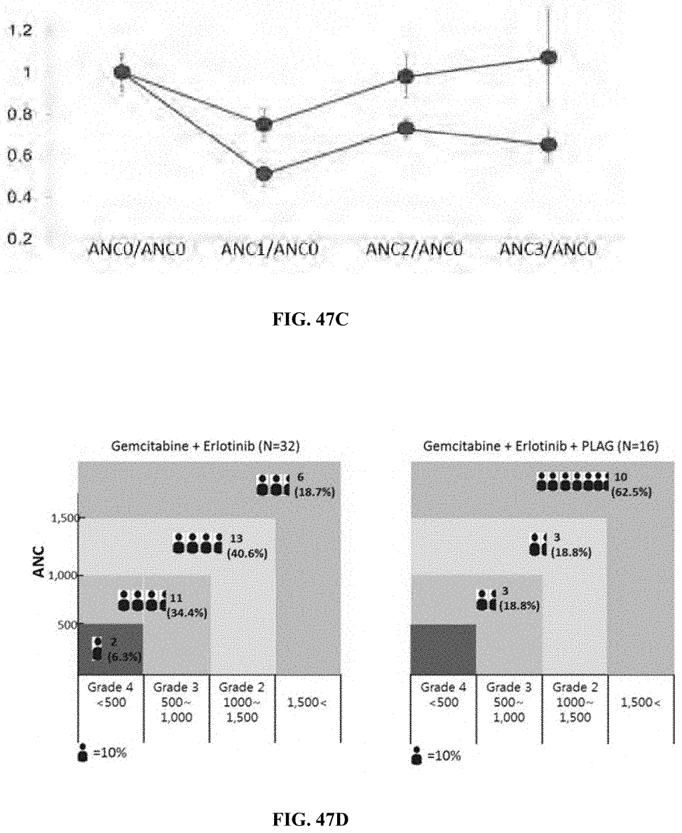

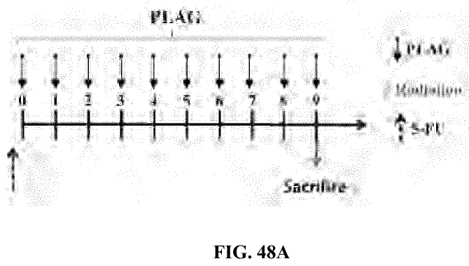

The profile of patients in the control group and the PLAG group is shown (FIG. 47A). Eight patients in the PLAG group and sixteen patients in the control group received two cycles of chemotherapy according to the schedule (FIG. 47B). Eight patients in the PLAG group and sixteen patients in the control group received three cycles of chemotherapy. In the PLAG group, PLAG 500 mg was orally administered twice daily from the start of the chemotherapy to the completion. For each cycle, the reduction percentage of ANC was evaluated in both groups. The ANCs of the PLAG group (blue) decreased significantly less from the baseline level (ANCO) compared to those of the control group (red) (P<0.05), and this significant difference in the reduction percentage of ANCs between the two groups was sustained throughout the course of chemotherapy (FIG. 47C). The incidence of neutropenia (ANC<1,500/mm³, grade 2-4) was significantly lower among patients who received PLAG, compared to the control group (37.5% vs. 81.3%, P<0.05) (FIG. 47D). Severe neutropenia (ANC<500/mm³, grade 4) developed only in the control group. The ANC nadir of the control group (red, about 0.5) was significantly lower than that of the PLAG group (blue, about 0.75). Febrile neutropenia (FN) did not occur in both groups. In the PLAG group, all patients completed the intake of PLAG during the study period. There were no adverse events related to PLAG during chemotherapy including nausea/vomiting, bone pain, fatigue, and liver dysfunction.

### Example 48 PLAG Attenuates Chemo-Radiation Induced Oral Mucositis (CRIOM)

Materials and Methods

Mice (7-9 weeks, Balb/c mice, KAIST) were administered intraperitoneally 5-FU (100 mg/kg, Sigma Aldrich). After 1 hr, mice head received 20 Gy using x-ray irradiatior (X-RAD 320, 1.8 Gy/min). Custom-made lead shields were used for mice to limit the radiation to the heads. PLAG (Enzychem Lifesciences Co.) was administered orally with 250 mg/kg once daily. The experimental design of the study was represented in schematic design. Mice were sacrificed 9 days after head-only radiation and the isolated tongues were stained 1% toluidine blue (TB, Sigma Aldrich). PLAG administrated mice were shown no mucositis and ulcer in tongues.

Results

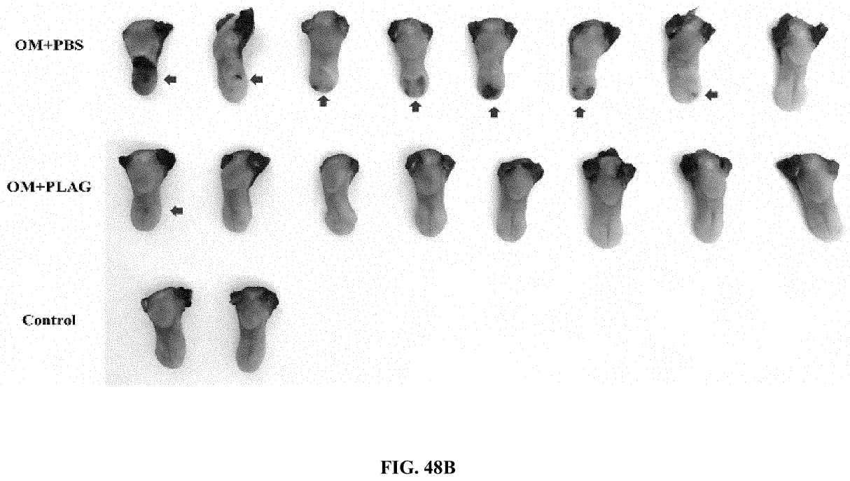

Mice were divided into three separate groups: 1) control (n=2), 2) radiated group (1Gy TBI of γ-radiation) with chemotherapy (5-FU) (n=8), and 3) PLAG co-administered group (n=8). Chemo-radiation induced oral mucositis (CRIOM) was induced after both γ-radiation (1Gy TBI of γ-radiation) and 5-FU treatment, and mice were sacrificed 9 days after the treatment (FIG. 48A). Toluidine blue stains ribonucleotides and detect inflammatory tissues. While seven out of eight mice tounges were stained with toluidine blue in the radiated group with chemotherapy, one out of eight mice tongues was stained in the PLAG treated group, as indicated by red arrows (FIG. 48B). This result indicates that PLAG is a promising pharmaceutical in attenuating oral mucositis induced by radiation and chemotherapy.

### Example 49 PLAG Attenuates Chemo-Radiation and Scratch Induced Oral Mucositis

Materials and Methods

Animal Experiments and Reagents

The 8 weeks female Balb/c mice were obtained from Koatech Co. (Pyongtaek, Republic of Korea) and preserved under fume hood conditions. Disposable gloves must be worn when handling animals. The bedding was changed once per week.

Oral Mucositis Experimental Models

For radiation therapy, mice received 1 Gy of gamma radiation with mice whole body. Mice were intraperitoneally administered 5-FU (Sigma Aldrich) at 50 mg/kg. For facilitating the risk of infection, mice were anesthetized with 2,2,2-tribromoethanol (150 mg/kg, Sigma Aldrich) by intraperitoneal injection, and then tongue was scratched 0.2 cm wound on a third of side at using the tip of an 18-gauge needle with an equal force and depth. PLAG (Enzychem Lifesciences) was stored in −80° C. refrigerator and was administered orally once with 250 mg/kg. Formulations were aggressively mixed to be cloudiness and were injected within 10 minutes.

Study Design

The tongue was scratched 0.2 cm wound on a third of side at using the tip of an 18-gauge needle with an equal force and depth on Day 0, 7, 10, and 16. Mice were received 1 Gy of gamma radiation on Day 2 and 5-FU was administered intraperitoneally in a dose of 50 mg/kg on Days 4. For the group receiving PLAG, 250 mg/kg of PLAG was administered orally once daily. To check the oral mucositis, mice were anesthetized with 2,2,2-tribromoethanol (150 mg/kg, Sigma Aldrich) by intraperitoneal injection on Day 7, 10, 14, 18. Each group contained seven mice. The details of the study design are shown in FIG. 57A.

Results

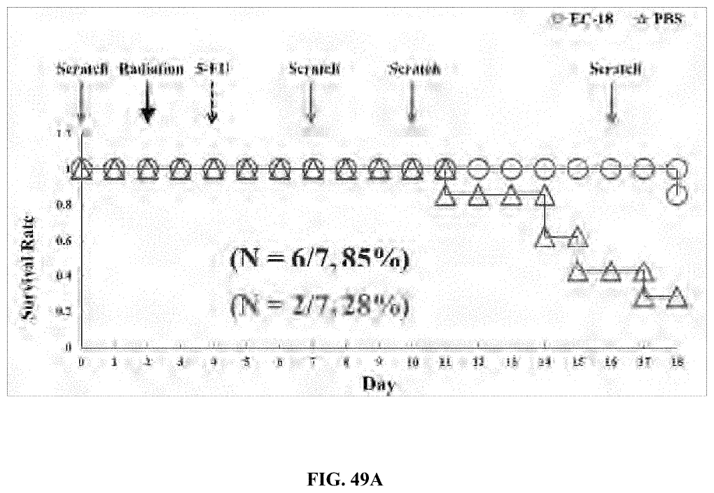

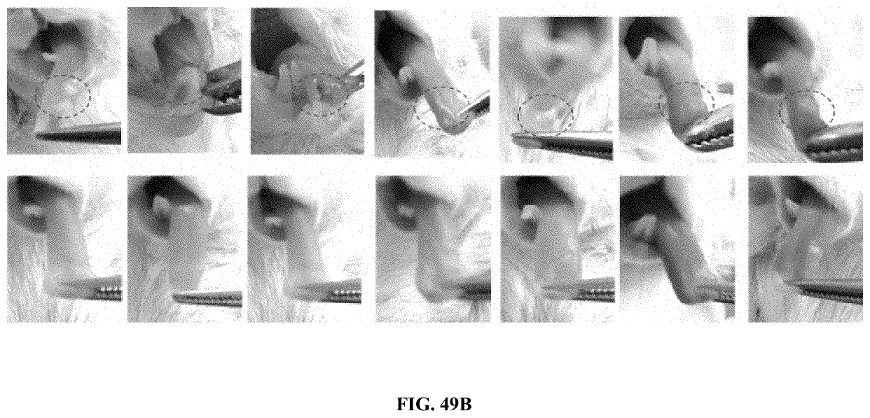

Mice were divided into two separate groups: 1) radiated group (1Gy TBI of γ-radiation) with chemotherapy (5-FU) and scratch (n=7) and 2) PLAG co-administered group (n=7). Oral mucositis was induced by treatment of γ-radiation (day 2), 5-FU (day 4), and slight scratch (days 0, 7, 10, and 16) (FIG. 49A). PBS was administered to the first group, and 250 mg/kg of PLAG was orally administered to the second group. While the first group showed 28% (=2/7) of survival rate after 18 days, the second group showed 85% (=6/7) of survival rate after 18 days. The first group (FIG. 49B, upper row) showed more severe CRIOM than the second group (FIG. 49B, lower row) did, as indicated by red circles. This result indicates that PLAG is a promising pharmaceutical in attenuating oral mucositis induced by radiation, chemotherapy and even scratch.

### Example 50 PLAG Attenuates Chemo-Radiation and PAK Induced Oral Mucositis

Materials and Methods

Animal Experiments and Reagents

Mice were obtained from Koatech Co. (Pyongtaek, Republic of Korea). Balb/c mice were 8 weeks old and preserved under specific pathogen-free conditions. The experiments were conducted with the approval of the Korea Research Institute of Bioscience and Biotechnology Institutional Review, Committee for Animal Care and Use (Daejeon, Republic of Korea). The mice were divided into 2 groups; CRIOM group and EC-18 group.

To induce immunocompromised condition in mice of CRIN group and EC-18 group, mice were administered by intraperitoneal injection with 30 mg/kg of 5-FU (Sigma) once a day for 3 days (Sigma) and five days after the experiment initiated, 1Gy of γ-radiation at once. For the EC-18 group, EC-18 (Enzychem Lifesciences, Daejeon, Republic of Korea) was orally given at a dose of 250 mg/kg once a day during the study period. The experimental schedule is described in FIG. 1. On day 6 after the experiment initiated, all the mice of 2 groups (CRIN group, and EC-18 group) were anesthetized with 2% 2, 2, 2,-tribromoethanol by intraperitoneal injection and infected with P. aeruginosa K (PAK) by syringe in the tongue. 1 day after PAK injection, the condition of the tongue and survival rate of mice were monitored and recorded.

P. aeruginosa K Strain Culture

P. aeruginosa K was cultured in LB broth or on LB agar plates overnight at 37° C. until they were in log-phase growth. Bacterial cells were harvested by centrifugation at 13,000×g for 2 min at 4° C. after overnight broth culture. The bacterial pellet was suspended to the appropriated number of colony-forming unit (CFU) per milliliter in PBS, as determined by optical density and plating out a serial dilution on broth agar plates. The bacteria were serially diluted to 2×10⁵ CFU in 50 μl PBS.

Survival Analysis

Chemoradiotherapy induced immunocompromised mice were challenged with PAK. 2 Groups of 19 male BALB/c mice (8-9 weeks old) were injected 5-FU 30 mg/kg once a day during 3 days by intravenous injection. The control group was additionally administered PBS and the experimental group was additionally administered EC-18 at 250 mg/kg every day through the oral. After 6 days, each mouse was infected 2×10⁵ CFU of PAK, suspended in 50 μl PBS, by syringe. After the challenge, mice were monitored for 1 day, and the survival rate of mice was recorded.

Results

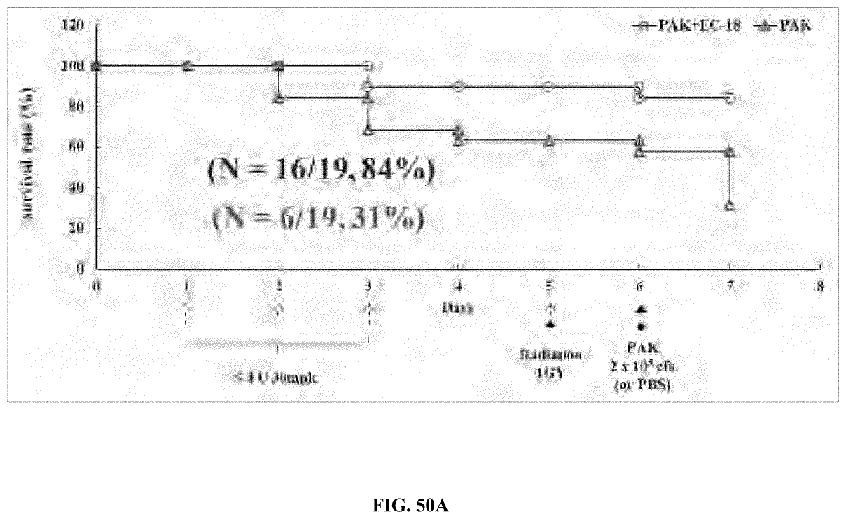

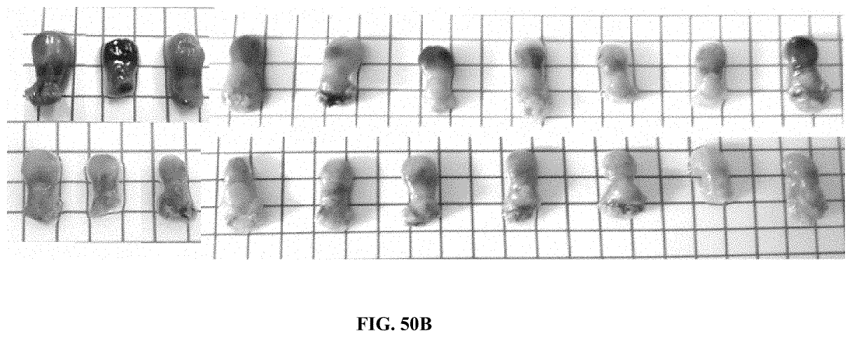

Mice were divided into two separate groups: 1) Pseudomonas aeruginosa (PAK) introduced mice with chemotherapy (5-FU) and radiation (1Gy TBI of γ-radiation) and 2) PLAG co-administered group (n=19). Oral mucositis was induced from DAMP molecules caused by treatment of γ-radiation (day 5), 5-FU (days 1-3), and PAK was introduced as PAMP molecules (day 6) (FIG. 50A). The combination of PAMP and DAMP molecules in this model induced severe and acute inflammation. While the first group showed 31% (=6/19) of survival rate after 7 days, the second group showed 84% (=6/19) of survival rate after 7 days. The phenotype of the first group (FIG. 50B, upper row) was more severe than the second group (FIG. 50B, lower row), as indicated by red circles. This result suggests that PLAG shows significant anti-inflammatory functions against bacteria and chemoradiation, thereby attenuating oral mucositis.

### Example 51 1-Palmitoyl-2-Linoleoyl-3-Acetyl-Rac-Glycerol Ameliorates Chemoradiation-Induced Oral Mucositis

This study was designed to investigate whether necroptosis is involved in the pathogenesis of chemoradiation-induced oral mucositis in a murine model and whether 1-palmitoyl-2-linoleoyl-3-acetyl-rac-glycerol (PLAG) ameliorates this disorder.

A chemoradiation-induced oral mucositis model was established by treating mice with concurrent 5-fluorouracil (100 mg/kg, i.p.) and head and neck X-irradiation (20 Gy). Phosphate-buffered saline or PLAG (100 mg/kg or 250 mg/kg, p.o.) was administered daily. Body weights were recorded daily, and mice were sacrificed on Day 9 for tongue tissue analysis.

On Day 9, chemoradiotherapy-treated (ChemoRT) mice had tongue ulcerations and experienced significant weight loss (Day 0:26.18±1.41 g; Day 9:19.44±3.26 g). They also had elevated serum macrophage inhibitory protein 2 (MIP-2) (control: 5.57±3.49 pg/ml; ChemoRT: 130.14±114.54 pg/ml) and inter-leukin (IL)-6 (control: 198.25±16.91 pg/ml; ChemoRT: 467.25±108.12 pg/ml) levels. ChemoRT-treated mice who received PLAG exhibited no weight loss (Day 0:25.78±1.04 g; Day 9:26.46±1.68 g) and had lower serum MIP-2 (4.42±4.04 pg/ml) and IL-6 (205.75±30.41 pg/ml) levels than ChemoRT-treated mice who did not receive PLAG. Tongue tissues of mice who received PLAG also displayed lower phos-phorylation levels of necroptotic signalling proteins. 1-Palmitoyl-2-linoleoyl-3-acetyl-rac-glycerol mitigated chemoradiation-induced oral mucositis by modulating necroptosis.

Oral mucositis is one of the most debilitating complications of common cancer treatments, such as chemotherapy and radiation therapy (Zhang et al., 2012). The overall occurrence of oral mucositis is over 90% in patients with head and neck cancer who received chemoradiotherapy (He et al., 2014; Muanza et al., 2005). Oral mucositis is characterized by acute inflammation and ulcerative lesions in the mucous membranes lining the mouth and throat (Al-Dasooqi et al., 2013; Maria, Eliopoulos, & Muanza, 2017; Sottili et al., 2018). Regardless of increased efforts for preventing the disorder, treatments are primarily limited to opioid analgesics for pain relief and an¬tibiotics for secondary bacterial infection (Im et al., 2019). Moreover, the mechanism and pathobiology of oral mucositis are not fully understood (Bertolini, Sobue, Thompson, & Dongari-Bagtzoglou, 2017).

Necroptosis is a form of programmed cell death with features of necrosis and apoptosis (Liu et al., 2018). It is an inflammatory cell death involving rapid plasma membrane permeabilization, leading to the release of cell contents and exposure of endogenous molecules, such as damage-associated molecular patterns (DAMPs) (Kaczmarek, Vandenabeele, & Krysko, 2013). Necroptosis occurs through activa¬tion of the necroptosis signalling axis, which includes receptor-interacting protein kinase 1 (RIPK1), receptor-interacting protein kinase 3 (RIPK3) and mixed lineage kinase domain-like pseudokinase (MLKL) (Barbosa et al., 2018a).

An increasing number of studies have suggested that necroptosis is associated with various acute injuries in different diseases (Zhao et al., 2015). Further, chemotherapy has been reported to promote inflammatory cell death of epithelial cells, and it has been suggested that necroptosis is induced via a positive feedback loop by elevated inflammatory cytokine levels produced by anti-cancer treatments (Xu et al., 2015). Moreover, an anti-necroptotic agent has shown protective effects against 5-fluorouracil (FU)-induced oral mucositis in a mouse model, acting through regulation of a DAMP known as high-mobility group box 1 (HMGB1) (Im et al., 2019). Therefore, in the current study, we decided to investigate whether necroptosis is associated with chemoradiation-induced oral mucositis.

Necroptotic cells passively release DAMPs. HMGB1 is the DAMP most commonly associated with oral mucositis (Tancharoen, Shakya, Narkpinit, Dararat, & Kikuchi, 2018; Vasconcelos et al., 2016). Interleukin (IL)-6 is also released as a sequela of necropto-sis and is known to initiate inflammation in other tissues (Deepa, Unnikrishnan, Matyi, Richardson, & Hadad, 2018; Zhao et al., 2015). IL-6 is an extensively studied proinflammatory cytokine in oral mu-cositis, and an anti-IL-6 monoclonal antibody has undergone clinical testing for the prevention of oral mucositis (Cinausero et al., 2017). One of the other major features of necroptosis is that it upregulates neutrophil chemoattractant. IL-8 is a chemotactic cytokine for neu-trophils, and it is upregulated when necroptosis occurs (de Oliveira et al., 2013; Zhu et al., 2018).

1-Palmitoyl-2-linoleoyl-3-acetyl-rac-glycerol (PLAG) is a mono-acetyl diacylglycerol that contains an acetyl group at the third posi¬tion of the glycerol backbone (Hwang et al., 2015; Jeong et al., 2016). PLAG has been studied for its anti-inflammatory effects and has ex¬hibited therapeutic efficacy against several inflammatory diseases (Kim et al., 2017; Ko et al., 2018). We previously showed that PLAG has therapeutic efficacy against chemotherapy- and scratching-in¬duced oral mucositis in murine models via modulating neutrophil migration (Lee et al., 2016). PLAG was also shown to downregulate several proinflammatory cytokines induced by oral mucositis.

In the current study, we examined whether necroptosis is a contributing factor to chemoradiation-induced oral mucositis and whether PLAG exhibited mitigating effects against this disorder. We established a murine model to accomplish these objectives, using body weight as an indicator of oral mucositis development and eval¬uating tongue tissues on a cellular and molecular level.

Materials and Methods

Mice and Housing

Male Balb/c mice (8-11 weeks old, 24-27 g) were purchased from the Korea Advanced Institute of Science and Technology (Daejeon, Republic of Korea) and maintained under specific pathogen-free conditions with free access to food and water. In each cage, 4 to 5 mice were housed together. After receiving approval from the Institutional Review Committee for Animal Care and Use of Korea Research Institute of Bioscience and Biotechnology (date of approval: 18 Jun. 2018; KRIBB-AEC-18158), all animal experiments were performed in accordance with the Guide for the Care and Use of Laboratory Animals. All experiments were conducted with 5 mice per group.

Establishing the Chemoradiation-Induced Oral Mucositis Mouse Model

On Day 0, mice were administered 100 mg/kg 5-FU (Sigma-Aldrich) or phosphate-buffered saline (PBS; WelGENE Inc.) via intraperito-neal (i.p.) injection. After 30 min, the mice were anesthetized with 2,2,2-tribromoethanol (Sigma-Aldrich) and received 20 Gy using an X-ray irradiator (X-RAD 320). Irradiation was fractionated: 10 Gy×2 with a 5-min break between fractions. Custom-made lead shields with a thickness of 0.5 cm were used to limit radiation to the head and neck area, with the mice placed in the supine position. The dose rate was 1.8 Gy per minute using 1.5-mm-thick Al filtration (300 kV), and the focus-to-skin distance was 40 cm.

1-Palmitoyl-2-linoleoyl-3-acetyl-rac-glycerol (1 mg/ml; Enzychem Lifesciences Corporation) was emulsified in PBS. Mice were administered 100 or 250 mg/kg body weight PLAG or PBS by oral gavage before 5-FU injection, and then daily at the same time of each day. After ChemoRT, mice were placed on a heated pad to recover and housed in a temperature- and light-controlled environment. Their body weights were recorded daily. As the ChemoRT-treated mice exhibited significant weight loss (approximately 20%) by Day 9, they were sacrificed on that day, and their tongues and blood samples were collected. No animal died before Day 9.

Toluidine Blue Staining and Histopathological Examination

Tongues harvested on Day 9 were stained for 1 min with 1% tolui-dine blue (TB; Sigma-Aldrich) in 10% acetic acid (EMSURE), followed by repeated washing with 10% acetic acid and PBS (Muanza et al., 2005). Macroscopic photographs were obtained from the dorsal view of tongues, and the stained areas were analysed using ImageJ software (National Institutes of Health, Maryland, USA). The ana¬lysed numbers were used to calculate the ulceration area percentage (ulcer area/total area×100%).

Measuring the Oral Mucosa Epithelial Thickness

On Day 9, the harvested tongues were fixed in 10% neutral buff¬ered formalin for 24 hr, embedded in paraffin, cut into 4-μm-thick sections, and stained with haematoxylin and eosin (H&E). Oral mu-cosa epithelial thickness was measured by viewing the H&E samples under a light microscope (Olympus). Epithelial thickness was meas¬ured from the basal membrane to the epithelial granular layer on the dorsal surface of each tongue section using the linear measure¬ment tool provided in NIS-Elements BR Ver4 (Nikon). The thickness was measured at 20 randomly selected sites in tissue slides, and the mean values (with standard deviation) were calculated (Ryu et al., 2010; Canard et al., 2008; Zheng et al., 2009).

Histopathologic Grading of Oral Mucositis

On Day 9, the H&E-stained tongue slides underwent histopathologi-cal grading of oral mucositis, based on a published study (Sunavala-Dossabhoy, Abreo, Timiri Shanmugam, & Caldito, 2015). A clinical pathologist blinded to the mouse's treatment graded the slides as follows: 0=no radiation injury (normal mucosa), 1=focal or diffuse alteration of basal cell layer with nuclear atypia and ≤2 dyskeratotic squamous cells, 2=epithelial thinning (2-4 cell layers) and/or ≥3 dys-keratotic squamous cells in the epithelium, 3a=loss of epithelium without a break in keratinization or the presence of atrophied eosino-philic epithelium, 3b=subepithelial vesicle or bullous formation, and 4=complete loss of epithelial and keratinized cell layers (ulceration).

Immunohistochemical Staining

To detect neutrophil infiltration, cytoplasmic translocation of HMGB1, and phosphorylated MLKL in the mouse tongues, the harvested samples were paraffin-embedded, cut into 4-μm-thick sections, and incubated overnight at 4° C. with anti-neutrophil antibody (NIMP-R14) (Invitrogen), anti-HMGB1 (Invitrogen) and anti-P-MLKL (Ser345) (Novus Biologicals, NBP2-66953, LLC). HRP-conjugated goat anti-rat IgG (Santa Cruz Biotechnology) and HRP-conjugated rabbit/mouse antibody (Dako) were then added, and the samples were incubated at room temperature for 15 min, followed by visualization with 3-amino-9-ethylcarbazole substrate (Dako). The tissues were then counterstained with 10% Mayer's haematoxylin (Dako), washed, dehydrated and mounted using Crystal Mount (Sigma-Aldrich). Photographic images were obtained of the dorsal surface of the tongue tissues, viewed under a light microscope (Olympus).

Enzyme-Linked Immunosorbent Assay

Concentrations of macrophage inflammatory protein 2 (MIP-2; the murine homologue of CXCL8) and IL-6 were measured in serum and tissue extracts. For tissue extracts, the tongues of each mouse were homogenized and lysed in an extraction buffer (20 mM Tris-HCl, pH 7.5, 150 mM NaCl, 1 mM EDTA, 1% Triton X-100 with protease and phos-phatase inhibitor cocktails) (Chen et al., 2018). Mouse MIP-2 and IL-6 ELISA kits (BD Bioscience) were used according to the instructions pro¬vided by the manufacturer. Optical densities were measured at 450 nm using an ELISA reader (Molecular Devices). Cytokine levels were calcu¬lated using a standard curve generated by a curve-fitting program.

Western Blotting

Mouse serum was used to detect circulating HMGB1 and heat shock protein 90 (Hsp90), another DAMP. Serum (3 μl) was diluted with 72 μl of 1×SDS sample buffer and heated at 98° C. for 5 min (Abdulahad et al., 2011). The samples were then loaded on 10% and 12% SDS-PAGE gels. Antibodies to HMGB1 (Abcam, ab18256) and Hsp90 (Santa Cruz Biotechnology, SC-13119) were used as the primary antibodies. The protein membrane was stained with Ponceau S solution (Sigma-Aldrich) to demonstrate comparable protein loading (Hwang et al., 2014). To detect the necroptosis sig¬nalling pathway, the tongues were homogenized and then lysed in RIPA buffer (LPS Solution) containing phosphatase and protease inhibitor cocktails (Sigma-Aldrich). The samples were loaded on 10% SDS-PAGE gels, and the following primary antibodies were ap-plied: phosphorylated (P)-RIP1 (Cell Signaling Technology #31122), RIPK1 (Abcam, ab72139), P-RIP3 (Thr231/Ser232) (CST, #57220), RIPK3 (Santa Cruz Biotechnology, SC-374639), P-MLKL (Ser345) (Novus Biologicals, NBP2-66953), MLKL (Biorbyt LLC; orb32399) and β-actin (CST, 8H10D10). This was followed by addition of sec¬ondary anti-rabbit and anti-mouse antibodies (ENZO Life Sciences).

RNA Isolation and Reverse Transcription Polymerase Chain Reaction

To To detect IL-6 and MIP-2 at the transcriptional level, total RNA was isolated from the mouse tongues using Tri-RNA Reagent (FAVORGEN Biotech), as specified by the manufacturer's instruc¬tions. RNA concentrations and qualities were measured using a NanoDrop device (Eppendorf BioSpectrometer). For cDNA synthe¬sis, 500 ng total RNA was reverse-transcribed using a primer (oligo-dT) and M-MLV reverse transcriptase (Promega). Conventional PCR was subsequently performed using Solg 2×h-Taq PCR Smart Mix (SolGent) and the Bio-Rad C1000 Touch Thermal Cycler (Bio-Rad Laboratories). The following MIP-2 and IL-6 primer sets were used: mouse CXCL2 forward, 5′-AGTGAACTGCGCTGTCAATG-3′ (SEQ ID NO. 1); mouse CXCL2 reverse, 5′-CTTTGGTTCTTCCTTGAGG-3′ (SEQ ID NO. 2); mouse IL-6 foward, 5′-GATGCTACCAAACTGGATA TAATC-3′ (SEQ ID NO. 13); and mouse IL-6 reverse, 5′-GGTCCTTAGCCACTCCTTCTGTG-3′ (SEQ ID NO. 14).

Statistical Analysis

Quantitative results are expressed as mean±standard error of the mean (SEM). All statistical analyses were performed using GraphPad Prism, version 5.01 (GraphPad Software Inc.). When comparing ser¬ially collected data, two-way repeated measures analysis of variance (ANOVA) was used. When analysing data collected at one time point, one-way ANOVA was used for comparisons between multiple groups, and Student's t test was used for comparisons between two experi¬mental groups. p values <0.05 were considered statistically significant.

Establishment of an X-Radiation and 5-FU-Induced Oral Mucositis Mouse Model

Based on previously published reports (Maria, Syme, Eliopoulos, & Muanza, 2016; Ryu et al., 2010; Zhao et al., 2009), we conducted a series of experiments using 5-FU and X-radiation to induce oral

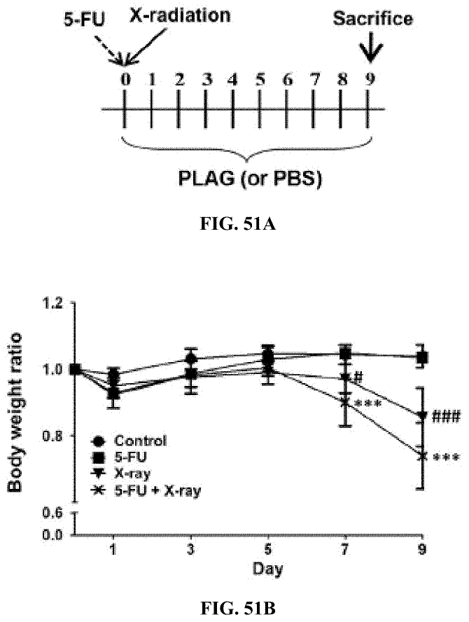

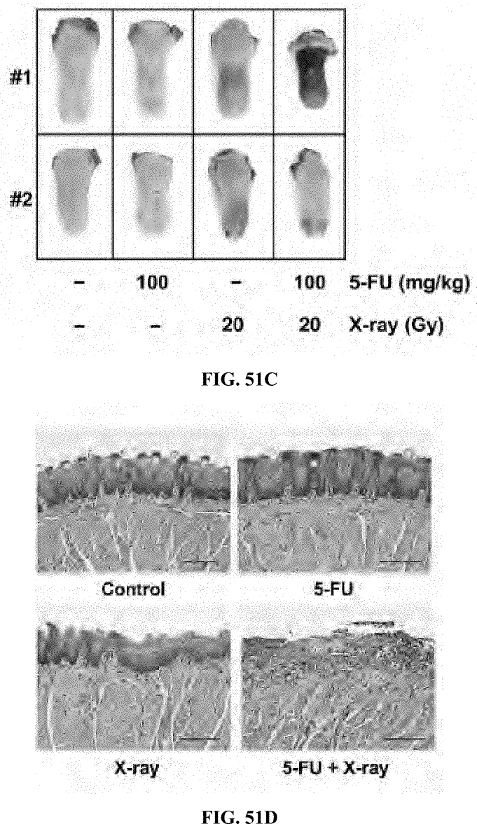

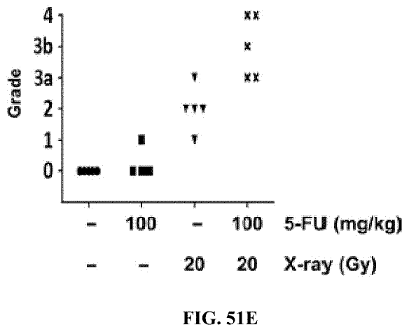

mucositis in a murine model. Accordingly, a chemoradiation-in-duced oral mucositis mouse model was established with the follow¬ing doses: 100 mg/kg 5-FU and 20 Gy X-radiation to the head and neck region (FIG. 51A). To characterize the model, we evaluated these four groups: control, 20 Gy, 5-FU, and ChemoRT (100 mg/kg 5-FU+20 Gy X-radiation). Changes in body weight were monitored and recorded daily, as they are an important indicator of the devel¬opment of mucositis in murine models and human patients (Al Jaouni et al., 2017; Co, Mejia, Que, & Dizon, 2016). Reduced dietary intake and poor absorption of nutrients secondary to difficulties with swal-lowing or inflammation of oral mucous membranes have been asso¬ciated with decreased body weight in murine models (Patel, Biswas, Shoja, Ramalingayya, & Nandakumar, 2014). All mice were sacrificed on Day 9 because the ChemoRT-treated mice had lost approximately 20% of their body weight by that time, necessitating euthanasia. As shown in FIG. 51B, the 20 Gy and ChemoRT groups exhibited sig¬nificant weight loss by Day 7, compared to Day 0, and the weight loss was more severe by Day 9 (Day 9 control: 26.50±3.10 g, p=0.30 vs. Day 0; Day 9 20 Gy: 21.82±0.85 g, p<0.001 vs. Day 0; Day 9 5-FU: 25.04±2.79 g, p=0.34 vs. Day 0; Day 9 ChemoRT: 23.62±2.87 g, p<0.001 vs. Day 0). FIG. 51C displays the harvested tongues stained with TB on Day 9. ChemoRT-treated mice exhibited the most se-vere changes, with prominent ulcers. FIG. 51D shows H&E stain¬ing of the dorsum of the harvested tongues. FIG. 51E illustrates the histopathological grading results for each treatment group. The ChemoRT group had the most severe histopathological changes, with the tongues from all mice graded as 3a or higher.

PLAG Attenuated Chemoradiation-Induced Oral Mucositis

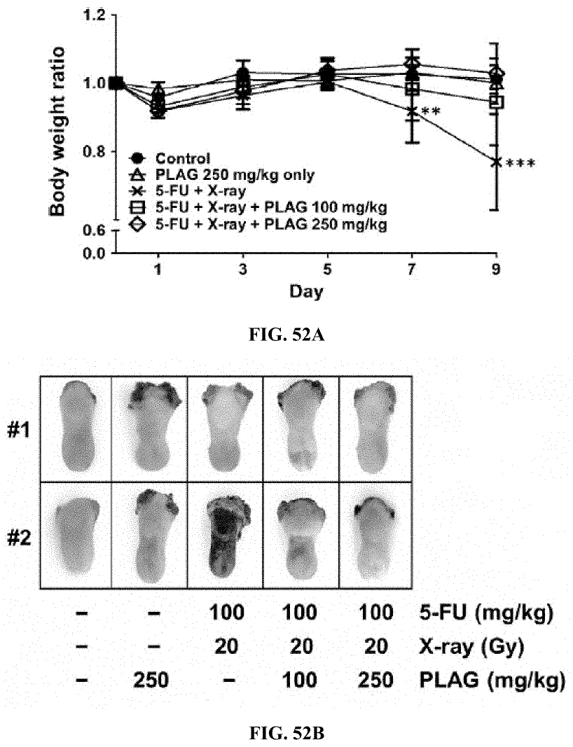

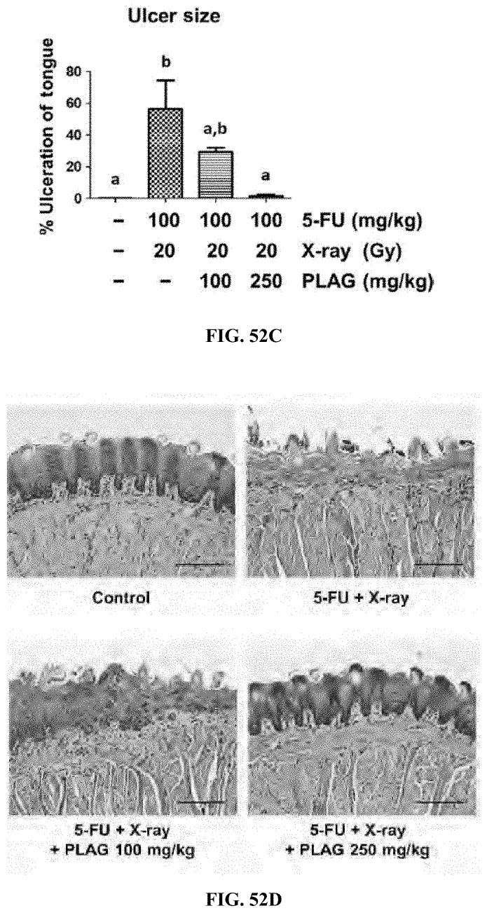

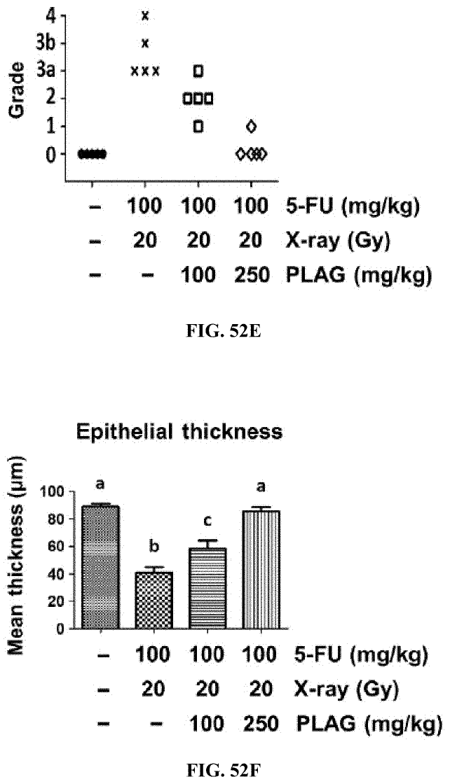

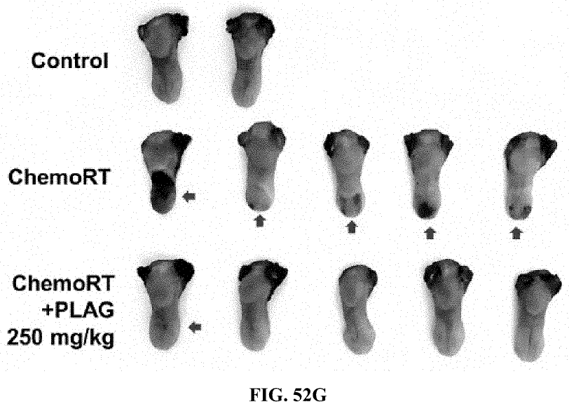

To investigate whether PLAG ameliorates chemoradiation-induced oral mucositis, different doses of PLAG were administered to the mice daily. As shown in FIG. 52A, no significant weight loss occurred from Day 0 to Day 9 in control mice or ChemoRT-treated mice who received 100 mg/kg or 250 mg/kg PLAG; by contrast, significant weight loss was observed in the ChemoRT-treated group who did not receive PLAG (Day 9 control: 25.72±1.23 g, p=0.38 vs. Day 0; Day 9 PLAG only: 25.66±0.70 g, p=0.35 vs. Day 0; Day 9 ChemoRT: 20.94±2.90 g, p<0.001 vs. Day 0; Day 9 ChemoRT+PLAG 100 mg/kg: 23.98±2.80 g, p=0.18 vs. Day 0; Day 9 ChemoRT+PLAG 250 mg/kg: 26.46±1.68 g, p=0.24 vs. Day 0). FIG. 52B displays the harvested tongues stained with TB on Day 9. The ChemoRT group developed ulcerations and erosions on their tongues, whereas the ChemoRT+PLAG mice exhibited fewer ulcerations.

We used these three markers to assess oral mucositis: ulceration area, histopathologic grading and oral mucosa epithelial thickness. ImageJ analysis showed that the ulceration area percentage was higher in the ChemoRT-treated mice receiving no PLAG than in the control mice (control: 0.17±0.13% and ChemoRT: 56.43±37.89%, p<0.01). By contrast, the ulceration area percentage was signifi¬cantly lower in the ChemoRT+250 mg/kg PLAG group than in the ChemoRT group (ChemoRT+PLAG 100 mg/kg: 29.32±5.40%, p=0.10 vs. ChemoRT; ChemoRT+PLAG 250 mg/kg: 1.45±2.36%, p<0.01 vs. ChemoRT) (FIG. 52C). H&E staining (FIG. 52D) and his-topathologic grading (FIG. 52E) showed that the tongues of the ChemoRT-treated mice who did not receive PLAG were the most severely injured. The tongues of all 5 mice in the ChemoRT group were graded as 3a, 3b or 4, whereas the tongues of all ChemoRT-treated mice who received 250 mg/kg PLAG were graded as 0 or 1.

Oral mucosa epithelial thickness was evaluated using H&E-stained tongues (FIG. 52F). The ChemoRT group had significantly thinner epithelium than the control group (control: 88.96±9.06 μm and ChemoRT: 41.01±17.82 μm, p<0.05). PLAG reduced ChemoRT-induced damage, as the epithelial thickness was greater in the ChemoRT-treated mice who received either dose of PLAG than in ChemoRT-treated mice who did not receive PLAG (ChemoRT+PLAG 100 mg/kg: 58.06±24.97 μm, p<0.05 and ChemoRT+PLAG 250 mg/kg: 85.81±12.24 μm, p<0.001, compared to the ChemoRT group).

Overall, the higher PLAG dose was associated with most prom¬inent anti-mucositis effects. Therefore, all subsequent exper¬iments were conducted by comparing the ChemoRT group with the ChemoRT+PLAG 250 mg/kg group (FIG. 52G).

PLAG Ameliorated Proinflammatory Cytokine Release and Neutrophil Infiltration

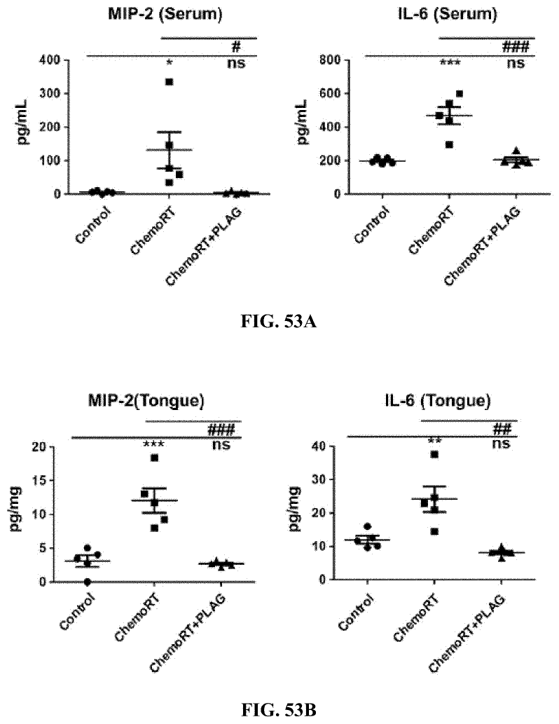

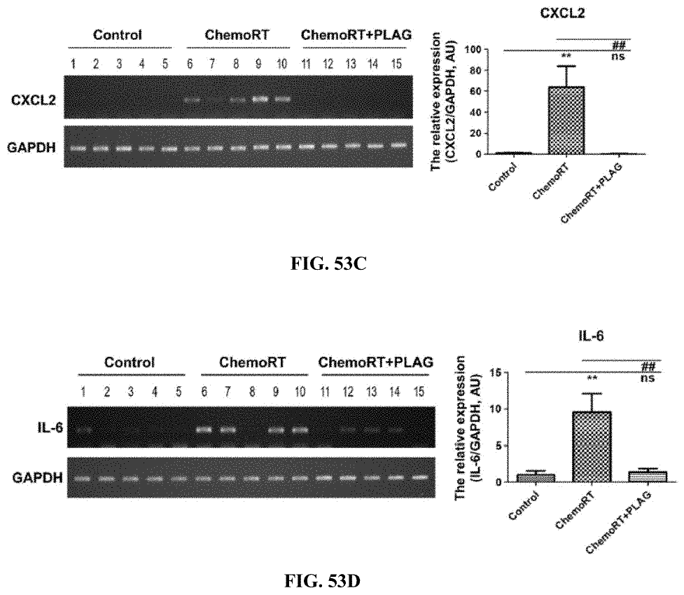

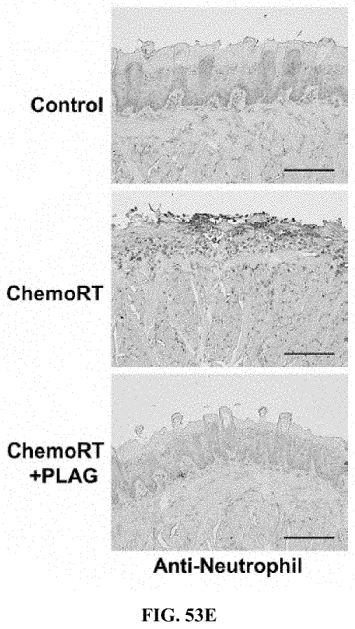

To determine the effects of oral mucositis on the inflammatory response, serum levels of proinflammatory cytokines were ex-amined by ELISA. FIG. 53A shows that on Day 9, the serum lev¬els of both MIP-2 and IL-6 were higher in the ChemoRT group than in the control group (MIP-2 control vs. ChemoRT: 5.57±3.49 pg/ml vs. 130.14±114.54 pg/ml, p<0.05; IL-6 control vs. ChemoRT: 198.25±16.91 pg/ml vs. 467.25±108.12 pg/ml, p<0.001). By contrast, ChemoRT-treated mice who received PLAG exhibited substantially less systemic inflammation than ChemoRT-treated mice who did not receive PLAG (MIP-2:4.42±4.04 pg/ml, p<0.05 vs. ChemoRT; IL-6:205.75±30.41 pg/ml, p<0.001 vs. ChemoRT).

To confirm whether the systemic inflammation in the ChemoRT group was caused by oral mucositis, cytokine levels in tongue-spe¬cific protein extracts were also measured. As shown in FIG. 53B, the findings were similar to those of the serum samples. M IP-2 and IL-6 levels in tongue tissue extracts were higher in the ChemoRT group than in the control group (MIP-2 control vs. ChemoRT: 3.07±1.78 pg/mg vs. 12.07±3.82 pg/mg, p<0.001; IL-6 control vs. ChemoRT: 11.97±2.39 pg/mg vs. 24.12±8.01 pg/mg, p<0.01). By contrast, mice receiving PLAG had lower MIP-2 and IL-6 lev¬els than those undergoing ChemoRT alone (MIP-2:2.69±0.38 pg/mg, p<0.001 vs. ChemoRT; IL-6:8.13±1.19 pg/mg, p<0.01 vs. ChemoRT).

CXCL2 expression and IL-6 mRNA expression in the mouse tongues were compared by calculating relative band intensities using ImageJ, with the values expressed in arbitrary units (AU). mRNA expression of both CXCL2 and IL-6 was elevated in the tongues of ChemoRT-treated mice, compared to the control mice (CXCL2 con¬trol vs. ChemoRT: 1.00±1.35 AU vs. 64.06±42.00 AU, p<0.01; IL-6 control vs. ChemoRT: 1.00±1.16 AU vs. 9.55±5.34 AU, p<0.01). Further, mRNA expression of both cytokines was downregulated in the PLAG group, compared with the ChemoRT (CXCL2: 0.23±0.48 AU, p<0.01 vs. ChemoRT; IL-6:1.34±1.06 AU, p<0.01 vs. ChemoRT) (FIGS. 53C and 53D).

To detect neutrophil infiltration in the oral epithelium, tissue slides were stained with the anti-neutrophil antibody NIMP-R14 for immunohistochemistry (IHC). The tongues of ChemoRT-treated mice who did not receive PLAG exhibited neutrophil recruitment in the oral epithelium due to elevated levels of MIP-2, whereas neutrophil infiltration was not observed in the tongues of ChemoRT-treated mice who received PLAG (FIG. 53E).

Release of DAMPs was Reduced by PLAG

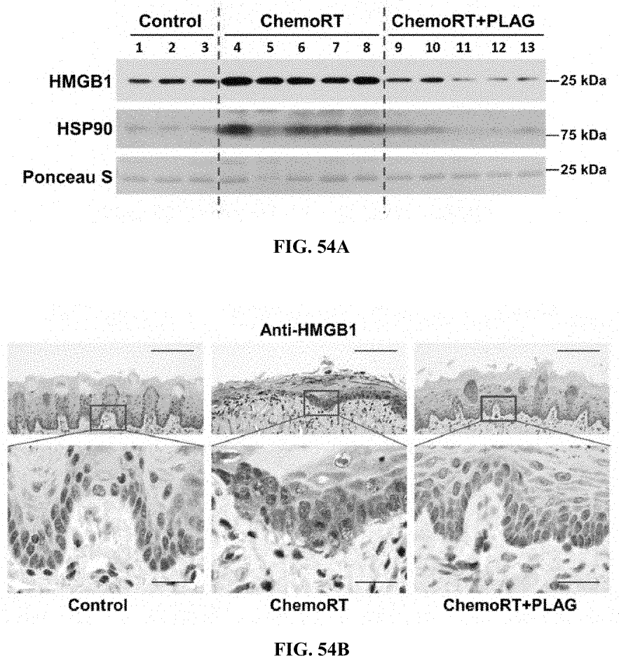

To further evaluate systemic inflammation and its relation to necrotic epithelium, serum levels of DAMPs were examined by Western blot¬ting. Serum levels of HMGB1 and Hsp90 were higher in the ChemoRT group than in the control group, but the levels of both DAMPs were similar between PLAG-treated and control mice (FIG. 54A). To de¬termine whether HMGB1 detected in the serum originated from the oral mucosa, we performed IHC by staining tongue tissue slides with anti-HMGB1 (Im et al., 2019). As shown in FIG. 54B, cytoplasmic HMGB1 was positively stained in the ChemoRT group, indicating that translocation of HMGB1 from the nucleus to the cytoplasm occurred in these mice. By contrast, HMGB1 remained in the nucleus in PLAG-treated mice.

PLAG Downregulates the Necroptosis Signalling Pathway

To further evaluate systemic inflammation and its relation to necrotic epithelium, serum levels of DAMPs were examined by Western blotting. Serum levels of HMGB1 and Hsp90 were higher in the ChemoRT group than in the control group, but the levels of both DAMPs were similar between PLAG-treated and control mice (FIG. 54A). To determine whether HMGB1 detected in the serum originated from the oral mucosa, we performed IHC by staining tongue tissue slides with anti-HMGB1 (Im et al., 2019). As shown in FIG. 54B, cytoplasmic HMGB1 was positively stained in the ChemoRT group, indicating that translocation of HMGB1 from the nucleus to the cytoplasm occurred in these mice. By contrast, HMGB1 remained in the nucleus in PLAG-treated mice.

PLAG Downregulates the Necroptosis Signalling Pathway

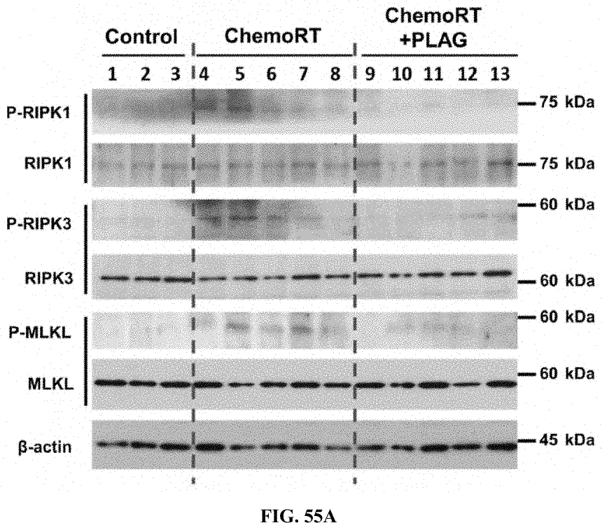

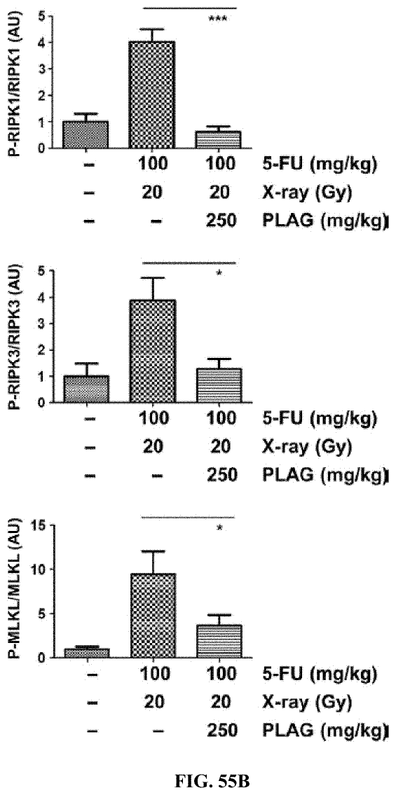

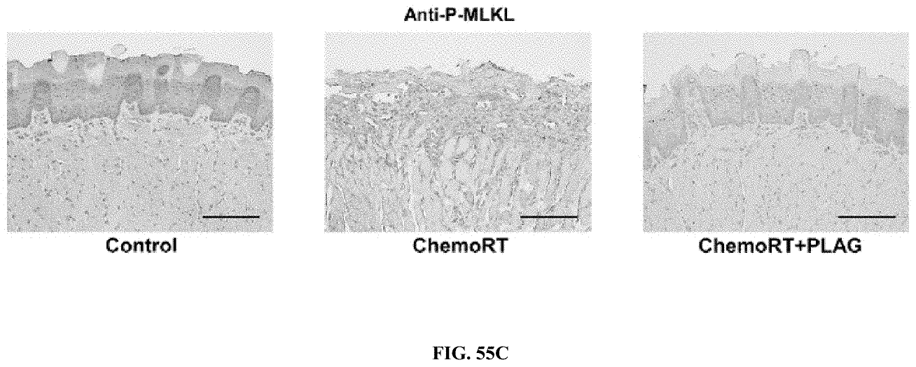

To assess whether the observed inflammatory responses were as¬sociated with necroptotic damage in the oral mucosa, the necrop-tosis signalling pathway was examined in tongue lysates using Western blotting (FIG. 55A). Relative band intensities were determined and compared between groups using Student's t test. The results showed that phosphorylation of RIPK1, RIPK3 and MLKL in the tongues of ChemoRT-treated mice was modulated by PLAG (P-RIPK1 control vs. ChemoRT vs. ChemoRT+PLAG: 1.00±0.45 AU vs. 4.02±1.02 AU vs. 0.61±0.45 AU, p<0.01 for ChemoRT vs. ChemoRT+PLAG; P-RIPK3 control vs. ChemoRT vs. ChemoRT+PLAG: 1.00±0.74 AU vs. 3.88±1.81 AU vs. 1.27±0.83 AU, p<0.05 for ChemoRT vs. ChemoRT+PLAG; P-MLKL control vs. ChemoRT vs. ChemoRT+PLAG: 1.00±0.47 AU vs. 9.48±5.45 AU vs. 3.67±2.56 AU, p<0.05 for ChemoRT vs. ChemoRT+PLAG) (FIG. 55B). These findings were verified by histological observations using IHC. Levels of P-MLKL in the oral mucosa epithelium and con¬nective tissues were higher in the ChemoRT group than in the con¬trol and PLAG-treated groups (FIG. 55C).

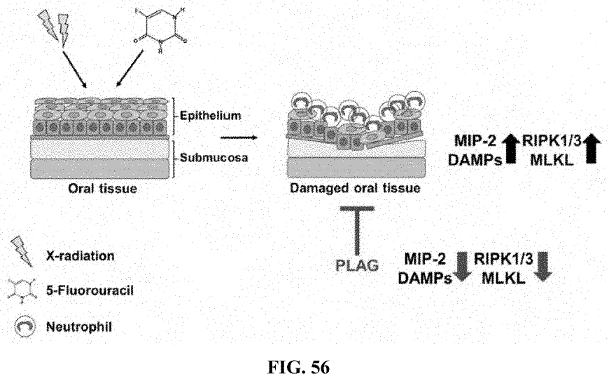

Based on our results, we propose a schematic for the pathogene¬sis of chemoradiation-induced oral mucositis and the role of PLAG (FIG. 56). By Day 9 after ChemoRT, mice exhibited oral mucositis as an acute response. DAMPs and proinflammatory cytokines were released from the damaged oral mucosa and led to systemic necro-inflammation via the circulatory system. In addition, neutrophils were recruited to the oral epithelium because of the elevated MIP-2 level and passively released DAMPs. Tongue tissues from ChemoRT-treated mice also exhibited activation of the necroptotic signalling axis, confirming that the inflammatory response was related to necroptosis. We also confirmed that PLAG ameliorated oral mu-cositis by lowering levels of proinflammatory cytokines and DAMPs through modulation of the necroptosis signalling pathway.

Effective early management of necroptosis is critical, as necro-ptosis can cause systemic inflammation, leading to damage in other tissues and thereby increasing the difficulty of successful treatment. During necroptosis of injured tissues (as can be induced by che¬motherapy or radiotherapy), neutrophils are recruited to eliminate DAMPs that may threaten normal tissues via autocrine and paracrine effects (Watts & Walmsley, 2018; Pouwels et al., 2016; Buisan et al., 2017; Handly, Pilko, & Wollman, 2015; Choi, Cui, Chowdhury, & Kim, 2017). The level of neutrophil recruitment at the site of oral lesions in mucositis correlates with the severity of histolog¬ical changes, including ulceration (Barbosa et al., 2018a; Lopes et al., 2010). Increased oral neutrophil infiltration is especially promi¬nent in 5-FU-induced oral mucositis (Barbosa et al., 2018b; Wright, Meierovics, & Foxley, 1986).

In addition to symptomatic treatment with analgesics and anti¬biotics for secondary infection, other treatment options currently available for oral mucositis include synthetic glucocorticoids (e.g. dexamethasone) and recombinant human keratinocyte growth fac¬tor (palifermin) (Lalla et al., 2014). Dexamethasone functions pri¬marily as an immunosuppressive agent, and palifermin stimulates epithelial cell proliferation. These two medications must be utilized with much consideration of the dosage and duration of treatment to prevent side effects and tumour cell growth (Riley et al., 2017). PLAG may be another potential preventive or treatment option for oral mucositis, providing a different treatment perspective by regulating necroptosis and the positive feedback loops involving DAMPs and proinflammatory cytokines.

Our results have shown that PLAG may have preventive ac-tivity against chemoradiation-induced oral mucositis, a common side effect of head and neck cancer therapy. Although no pub-lished studies have directly examined the relationship between head and neck cancer therapy and PLAG, a recent study evaluated the effects of PLAG on gemcitabine-induced neutropenia in a mice model (Jeong et al., 2019). According to that study, PLAG attenu¬ated the neutropenia and did not interfere with the anti-cancer ef¬fect of gemcitabine in athymic nude mice implanted with a human myeloma cell line. Therefore, we expect that PLAG may ameliorate oral mucositis caused by cancer therapy without interfering with treatment efficacy in patients with head and neck cancer.

In conclusion, chemoradiotherapy led to necroptosis of the tongue by Day 9 in our mouse model. Release of DAMPs and proin-flammatory cytokines from oral mucosa cells and subsequent neu-trophil infiltration into the oral epithelium were observed. PLAG ameliorated chemoradiation-induced oral mucositis by modulating the necroptosis signalling pathway. Based on these observations, we suggest that PLAG may be a useful option for preventing or treating chemoradiation-induced oral mucositis.

1. A method of modulating an inflammatory response by a cell comprising: administering to the cell a composition comprising a monoacetyl diacylglycerol compound, wherein the administration decreases expression of one or more cytokines, one or more chemokines, or a combination thereof.

2. The method of claim 1, wherein the cell is a eukaryotic cell.

3. The method of claim 2, wherein the eukaryotic cell is a human cell.

4. The method of claim 3, wherein the human cell is a macrophage.

5. The method of claim 1, wherein the one or more cytokines or chemokines comprises CXCL8, CXCL2, or IL-6.

6. The method of claim 1, wherein the administration further decreases release of one or more damage-associated molecular pattern (DAMP) molecules.

7. The method of claim 1, wherein the administration further increases trafficking of one or more pattern recognition receptors (PRRs) to a plasma membrane.

8. The method of claim 7, wherein the one or more PRRs is selected from the group consisting of a damage-associated molecular pattern receptor, a pathogen-associated molecular pattern receptor, a toll-like receptor, a G protein-coupled receptor, a C-type lectin receptor, or a combination thereof.

9. The method of claim 8, wherein the G protein-coupled receptor comprises one or more of rhodopsin-like G Protein-coupled receptors, secretin family receptor proteins, metabotropic glutamate receptors, fungal mating pheromone receptors, cyclic AMP receptors, and frizzled/smoothened G Protein-coupled receptors.

10. The method of claim 8, wherein the G protein-coupled receptor comprises a purinergic G protein-coupled receptor.

11. The method of claim 9, wherein the purinergic G protein-coupled receptor is a P2Y1, P2Y2, P2Y4, P2Y6, P2Y11, P2Y12, P2Y13 or P2Y14 receptor.

12. The method of claim 1, wherein modulating the inflammatory response treats a disease or disorder in a subject in need thereof.

13. The method of claim 12, wherein the disease is pneumonia.

14. The method of claim 1, wherein the monoacetyl diacylglycerol compound modulates a scavenger receptor.

15. The method of claim 14, wherein the scavenger receptor is a scavenger receptor type A.

16. The method of claim 15, wherein the monoacetyl diacylglycerol compound binds to the scavenger receptor type A.

17. The method of claim 1, wherein the monoacetyl diacylglycerol compound is a compound of Formula I: 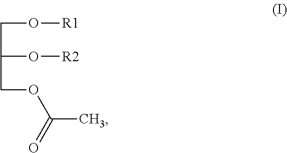
wherein R1 and R2 are independently a fatty acid group comprising 14 to 20 carbon atoms.

18. The method of claim 17, wherein the monoacetyl diacylglycerol compound is a compound of Formula II:

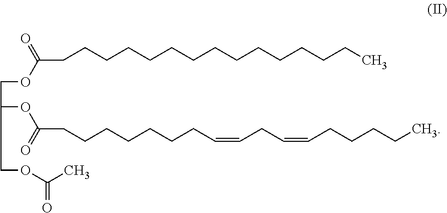 [SMILES: CCCCC/C=C\C/C=C\CCCCCCCC(=O)OC(COC(C)=O)COC(=O)CCCCCCCCCCCCCCC]

19-61. (canceled)

62. A method for treating a subject suffering from or susceptible to pneumonia, comprising administering to the subject a monoacetyl diacylglycerol compound of Formula (I): 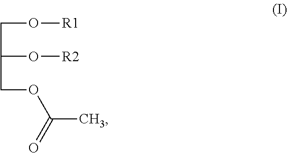
wherein R1 and R2 are independently a fatty acid group comprising 14 to 20 carbon atoms.

63-69. (canceled)


SEQUENCE LISTING (14 sequences)

SEQ ID NO: 1 - DNA, 20 bp, Artificial Sequence
  Primer
agtgaactgcgctgtcaatg

SEQ ID NO: 2 - DNA, 20 bp, Artificial Sequence
  Primer
ctttggttcttccgttgagg

SEQ ID NO: 3 - DNA, 20 bp, Artificial Sequence
  Primer
atgccgtctgaactggagaa

SEQ ID NO: 4 - DNA, 20 bp, Artificial Sequence
  Primer
tgctactccttgtggctgtc

SEQ ID NO: 5 - DNA, 19 bp, Artificial Sequence
  Primer
atggccaacaaagcacctt

SEQ ID NO: 6 - DNA, 20 bp, Artificial Sequence
  Primer
ttacttcccacagcctttgc

SEQ ID NO: 7 - DNA, 20 bp, Artificial Sequence
  Primer
ccatcaccatcttccaggag

SEQ ID NO: 8 - DNA, 20 bp, Artificial Sequence
  Primer
acagtcttctgggtggcagt

SEQ ID NO: 9 - DNA, 20 bp, Artificial Sequence
  Primer
gggctctgcctgagttgtag

SEQ ID NO: 10 - DNA, 20 bp, Artificial Sequence
  Primer
gtcaggccagctgaagtagg

SEQ ID NO: 11 - DNA, 20 bp, Artificial Sequence
  Primer
agggttgccagatgcaatac

SEQ ID NO: 12 - DNA, 20 bp, Artificial Sequence
  Primer
gtggatcctggctagcagac

SEQ ID NO: 13 - DNA, 24 bp, Artificial Sequence
  Primer
gatgctaccaaactggatataatc

SEQ ID NO: 14 - DNA, 23 bp, Artificial Sequence
  Primer
ggtccttagccactccttctgtg